(19) United States
(12) Patent Application Publication
(10) Pub. No.: US 2014/0221369 A1
(43) Pub. Date: Aug. 7, 2014

(54) 5-DEUTERO-2,4-THIAZOLIDINEDIONE AND 5-DEUTERO-2,4-OXAZOLIDINEDIONE DERIVATIVES AND COMPOSITIONS COMPRISING AND METHODS OF USING THE SAME

(71) Applicant: DeuteRx, LLC, Andover, MA (US)

(72) Inventor: Sheila DeWitt, Auburn, NH (US)

(73) Assignee: DEUTERX, LLC, Andover, MA (US)

(21) Appl. No.: 14/169,380

(22) Filed: Jan. 31, 2014

Related U.S. Application Data
(60) Provisional application No. 61/786,054, filed on Mar. 14, 2013.
(60) Provisional application No. 61/759,446, filed on Feb. 1, 2013.

(51) Int. Cl. C07D 417/12; C07D 417/14; C07D 471/04

(52) U.S. Cl. CPC C07D 417/12; C07D 471/04; C07D 417/14
USPC 514/234.5; 548/181; 514/369; 546/199; 514/322; 544/133; 546/118; 514/303; 546/269.7; 514/338

(57) ABSTRACT

The invention provides 5-deuterium enriched 2,4-thiazolidinediones and 2,4-oxazolidinediones, such as 5-(4-((6-(4-amino-3,5-dimethylphenoxy)-1-methyl-1H-benzo[d]imidazol-2-yl)methoxy)benzyl)-5-deutero-thiazolidine-2,4-dione, deuterated derivatives thereof, stereoisomers thereof, pharmaceutically acceptable salt forms thereof, and methods of treating medical disorders, such as cancer, using the same.

### RELATED APPLICATIONS

This application claims the benefit of and priority to U.S. Provisional Patent Application Ser. No. 61/759,446, filed Feb. 1, 2013, and U.S. Provisional Patent Application Ser. No. 61/786,054, filed Mar. 14, 2013; the contents of each of which are hereby incorporated by reference.

### BACKGROUND

Compounds such as 5-(4-((6-(4-amino-3,5-dimethylphenoxy)-1-methyl-1H-benzo[d]imidazol-2-yl)methoxy)benzyl)thiazolidine-2,4-dione (Formula A below) (inolitazone or efatutazone (CS-7017)) are peroxisome proliferator-activated receptor gamma (PPARγ) agonists currently being studied for their anticancer activity.

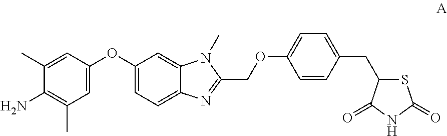 [SMILES: Cc1cc(Oc2ccc3nc(COc4ccc(CC5SC(=O)NC5=O)cc4)n(C)c3c2)cc(C)c1N]

The above compound is described in U.S. Pat. Nos. 6,432,993 and 8,263,631 and in International Patent Application Publication No. WO 2008/099944; the contents of which are hereby incorporated by reference.

The compound of Formula A, because of its asymmetrical 5-carbon on the 2,4-thiazolidinedione ring is a racemic mixture of R and S stereoisomers. The hydrogen at the 5-position is acidic due to the presence of the adjacent carbonyl moiety, thereby making it difficult to prevent racemization of the two stereoisomers and difficult to determine if one of the stereoisomers is superior to the other.

Despite the clinical interest in efatutazone, there is still a need for anti-cancer agents with improved properties. The invention provides new compounds that are resistant to racemization at their stereogenic center, and are useful in the treatment of various medical disorders.

### SUMMARY

The invention provides deuterium-enriched 2,4-thiazolidinediones and 2,4-oxazolidinediones, pharmaceutical compositions, and methods of treating medical disorders using the deuterium-enriched compounds and pharmaceutical compositions containing such deuterium-enriched compounds. The deuterium-enriched compounds contain deuterium enrichment at the chiral center of the thiazolidine-2,4-dione moiety and optionally in other locations in the compound. One aspect of the invention provides the deuterium-enriched compounds in enantiomerically pure form. The deuterium-enriched compounds described herein provide for a better therapeutic agent than non-deuterated versions of these compounds.

Accordingly, one aspect of the invention provides 5-deuterium enriched 2,4-thiazolidinediones and 2,4-oxazolidinediones (e.g., 5-(4-((6-(4-amino-3,5-dimethylphenoxy)-1-methyl-1H-benzo[d]imidazol-2-yl)methoxy)benzyl)-5-deutero-thiazolidine-2,4-dione) and stereoisomers and pharmaceutically acceptable salts thereof. The deuterium-enriched compounds are described by generic and specific chemical formulae. One aspect of the invention provides a deuterium-enriched compound represented by formula I:

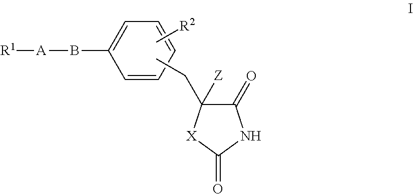

or a pharmaceutically acceptable salt or stereoisomer thereof, wherein the variables are as defined in the detailed description. A more specific embodiment of the invention provides a deuterium-enriched compound represented by formula XII:

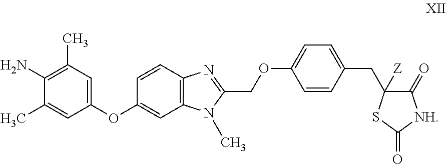

or a pharmaceutically acceptable salt or stereoisomer thereof, wherein the variables are as defined in the detailed description.

Another aspect provided herein is a pharmaceutical composition comprising a pharmaceutically acceptable carrier and at least one of the deuterium-enriched compounds described herein.

Also provided herein are methods for treating medical disorders. Exemplary medical disorders include, for example, cancer, neurological disorders, respiratory disorders, metabolic disorders, inflammatory disorders, cardiovascular disorders, dermatological disorders, and the like. Exemplary cancers include, for example, lung cancer, hepatocellular carcinoma, astrocytoma, glioma, glioblastoma, meningioma, liver cancer, lymphoma, melanoma, multiple myeloma, pancreatic cancer, colorectal cancer, pituitary cancer, thyroid cancer, esophageal cancer, and prostate cancer. Exemplary metabolic disorders include, for example, diabetes, insulin resistance, gout, hyperglycemia, nonalcoholic steatohepatitis, nonalcoholic fatty liver disease, diabetic retinopathy, diabetic neuropathy, diabetic nephropathy, beta-cell depletion insulin resistance in a patient with congenital adrenal hyperplasia treated with a glucocorticoid, dysmetabolism in peritoneal dialysis patients, reduced insulin secretion, improper distribution of brown fat cells and white fat cells, obesity, and improper modulation of leptin levels. Exemplary neurological disorders include, for example, Alzheimer's disease, Parkinson's disease, amyotrophic lateral sclerosis, Friedreich's ataxia, autism spectrum disorder, depression, mild cognitive impairment, neurodegeneration, adrenoleukodystrophy, Huntington's disease, stroke, traumatic brain injury, substance abuse, spinal cord injury, neuronal injury, and major depression or bipolar disorder comorbid with metabolic syndrome. The methods for treating medical disorders comprise administering to a patient in need thereof a therapeutically effective amount of a deuterium-enriched compound described herein, such as a compound of formula I, XII, XV, or XVIII, to treat the disorder.

A more specific embodiment of the therapeutic methods involves treating a disorder selected from the group consisting of cancer, diabetes, fatty liver disease, and cardiovascular disease in a patient, where the method comprises administering to a patient in need thereof a therapeutically effective amount of a deuterium-enriched compound described herein to treat the disorder. Yet another more specific embodiment of the therapeutic methods involves a method for the prophylaxis or treatment of a person having a carcinoma, sarcoma or hematopoietic cancer, where the method comprises administering to a person in need thereof a therapeutically effective amount of at least one of the deuterium-enriched compound described herein.

Still another more specific embodiment of the therapeutic methods involves a method for the prophylaxis or treatment of a person having a carcinoma, sarcoma or hematopoietic cancer, where the method comprises administering to a person in need thereof a therapeutically effective amount of at least one of the deuterium-enriched compounds described herein in conjunction with at least one drug selected from an epidermal growth factor receptor (EGFR) inhibitor, a vascular endothelial growth factor receptor (VEGFR) inhibitor, and a Raf kinase inhibitor.

Another aspect provided herein are novel deuterium-enriched compounds described herein for use in therapy. Also provided herein is the use of novel deuterium-enriched compounds described herein for the manufacture of a medicament.

These and other aspects, which will become apparent during the following detailed description, have been achieved by the inventor's discovery of 5-deuterium enriched 2,4-thiazolidinediones and 2,4-oxazolidinediones.

### BRIEF DESCRIPTION OF THE FIGURES

FIG. 1 is a graph showing in vitro stability data for (+)-5-[(p-{[6-(4-amino-3,5-xylyloxy)-1-methyl-1,3-diaza-1H-inden-2-yl]methoxy}phenyl)methyl]-1,3-thiazolidine-2,4-dione (designated “h+”) in human plasma, as described in Example 4, where the abbreviation “calc” indicates results from fitting experimental data to kinetic differential equations.

FIG. 2 is a graph showing in vitro stability data for (−)-5-[(p-{[6-(4-amino-3,5-xylyloxy)-1-methyl-1,3-diaza-1H-inden-2-yl]methoxy}phenyl)methyl]-1,3-thiazolidine-2,4-dione (designated “h−”) in human plasma, as described in Example 4, where the abbreviation “calc” indicates results from fitting experimental data to kinetic differential equations.

FIG. 3 is a graph showing in vitro stability data for 5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione (which is a mixture of the (−)-deuterated enantiomer (i.e., “d−”) and (+)-deuterated enantiomer (i.e., “d+”) and which is designated “d-rac”) in human plasma, as described in Example 4. The abbreviation “calc” indicates results from fitting experimental data to kinetic differential equations.

FIG. 4 is a graph showing PPARγ agonist activity of (−)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione (i.e., “d−”) and (+)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione (i.e., “d+”) as a function of concentration, as described in Example 5.

### DETAILED DESCRIPTION

Deuterium (D or ²H) is a stable, non-radioactive isotope of hydrogen and has an atomic weight of 2.014. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their non-enriched counterparts. Thus, the invention relates to a deuterium-enriched compound or compounds whose enrichment is greater than naturally occurring deuterated molecules.

All percentages given for the amount of deuterium present are mole percentages. Further, when a variable is not accompanied by a definition, the previous definition of the variable controls.

Unless indicated otherwise, when a D is specifically recited at a position or is shown in a formula, this D represents a mixture of hydrogen and deuterium where the amount of deuterium is about 100% (i.e., the abundance of deuterium is from 90% to 100%). In certain aspects, the abundance of deuterium is from 97% to 100%.

The 5-deuterium group (i.e., the Z group (or D)) in the present compounds means that the compounds have been isotopically enriched at the 5-position and are different and distinct from the corresponding non-enriched compound.

Compound refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

### I. EXEMPLARY DEUTERIUM-ENRICHED COMPOUNDS

One aspect of the invention provides a deuterium-enriched compound of formula I:

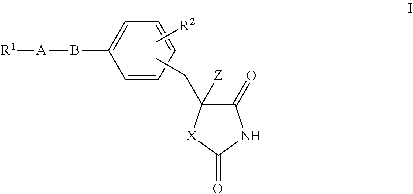

and pharmaceutically acceptable salts and stereoisomers thereof, wherein:

A is C₁₋₆ alkylene;

B is O or S;

X is O or S;

Z is H or D, provided that the abundance of deuterium in Z is at least 30%;

R¹ is selected from:

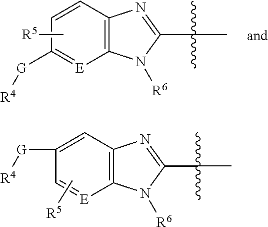

R² is selected from H; D; and R^{a};

E is CH or N;

G is O or S;

R⁴ is phenyl substituted with 1-5 R^{a}, or R⁴ is pyridyl substituted with 1-4 R^{a};

R⁵ is selected from H; D; and R^{a};

R⁶ is selected from H; D; C₁₋₆ alkyl; C₆₋₁₀ aryl group optionally substituted with 1-3 R^{b}; and C₇₋₁₆ aralkyl optionally substituted with 1-3 R^{b};

R^{a} is independently, at each occurrence, selected from:
a. halo;
b. hydroxyl;
c. C₁₋₆ alkyl;
d. halo-C₁₋₆ alkyl;
e. C₁₋₆ alkoxy;
f. C₁₋₆ alkylthio;
g. NH₂ optionally substituted with 1-2 R^{c};
h. C₃₋₁₀ cycloalkyl optionally substituted with 1-3 R^{b};
i. C₆₋₁₀ aryl optionally substituted with 1-3 R^{b};
j. C₇₋₁₆ aralkyl optionally substituted with 1-3 R^{b};
k. C₆₋₁₀ aryloxy optionally substituted with 1-3 R^{b};
l. C₇₋₁₆ aralkyloxy optionally substituted with 1-3 R^{b};
m. C₆₋₁₀ arylthio optionally substituted with 1-3 R^{b};
n. C₁₋₇ aliphatic acyloxy;
o. 4-7 membered saturated nitrogen-containing heterocyclic group;
p. 5-6-membered aromatic nitrogen-containing heterocyclic group;
q. NO₂; and
r. —CN;

R^{b} is independently, at each occurrence, selected from:
a. halo;
b. hydroxyl;
c. C₁₋₆ alkyl;
d. halo-C₁₋₆ alkyl;
e. C₁₋₆ alkoxy;
f. NH₂ optionally substituted with R^{c};
g. C₆₋₁₀ aryl; and
h. NO₂;

R^{c} is independently, at each occurrence, selected from:
a. C₁₋₁₀ alkyl optionally substituted with 1-3 groups R^{d};
b. C₆₋₁₀ aryl optionally substituted with 1-3 groups R^{d};
c. C₇₋₁₆ aralkyl optionally substituted with 1-3 groups R^{d};
d. C₁₋₇ aliphatic acyl optionally substituted with 1-3 groups R^{d};
e. C₇₋₁₁ aromatic acyl optionally substituted with 1-3 groups R^{d};
f. C₈₋₁₂ aromatic aliphatic acyl optionally substituted with 1-3 groups R^{d};
g. C₄₋₁₁ cycloalkylcarbonyl optionally substituted with 1-3 groups R^{d}; and,
h. 5-6 membered aromatic nitrogen-containing heterocyclic carbonyl group optionally substituted with 1-3 groups R^{d};

R^{d} is selected from: halogen; hydroxyl; C₁₋₆ alkyl; halo-C₁₋₆ alkyl; C₁₋₆ alkoxy; and C₁₋₆ alkylthio; and

a hydrogen atom present anywhere in the compound of Formula I is optionally replaced by D.

In certain embodiments, the deuterium-enriched compound is one of the generic formulae described herein wherein the abundance of deuterium in Z is selected from: (a) at least 40%, (b) at least 50%, (c) at least 60%, (d) at least 70%, (e) at least 80%, (f) at least 90%, (g) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

Deuterium-enriched compounds characterized according to their stereochemical purity are provided. The stereochemical purity of compounds having one stereocenter can be characterized as enantiomeric excess (ee). Enantiomeric excess (for the most abundant enanteriomer) can be calculated using the formula (when the (R) enantiomer is the most abundant):

ee(%)=(R−S)/(R+S)*100

where R and S are the amounts of (R) and (S) enantiomers in the mixture.

For compounds having two or more stereocenters, the stereochemical purity (sp) refers to the percentage of 1 of the 4 or more possible stereoisomers being present. For a compound with two stereocenters, the stereomeric purity can be calculated using the formula:

sp(%)=% Isomer 1−(% Isomer 2+% Isomer 3+% Isomer 4)

where % Isomer # is the weight (e.g., mole) % of one of the isomers in the mixture.

In another aspect, the invention provides a compound having an enantiomeric excess, with respect to the C—Z carbon, of at least 5%. Exantiomeric excess, with respect to the C—Z carbon (i.e., 5-carbon of the thiazolidinedione), refers only to the stereomeric purity around this carbon, regardless of whether or not additional stereocenters are present in the compound.

In another aspect, the invention provides deuterium-enriched compounds wherein the enantiomeric excess is selected from: (a) at least 10%, (b) at least 20%, (c) at least 30%, (d) at least 40%, (e) at least 50%, (f) at least 60%, (g) at least 70%, (h) at least 80%, (i) at least 90%, (j) at least 95%, (k) at least 97%, (l) at least 98%, and (m) at least 99%. Additional examples include an enantiomeric excess of at least 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

In another aspect, the invention provides a compound having stereomeric purity of at least 5%.

In another aspect, the invention provides deuterium-enriched compounds wherein the stereomeric purity is selected from: (a) at least 10%, (b) at least 20%, (c) at least 30%, (d) at least 40%, (e) at least 50%, (f) at least 60%, (g) at least 70%, (h) at least 80%, (i) at least 90%, (j) at least 95%, (k) at least 97%, (l) at least 98%, and (m) at least 99%. Additional examples of the stereoisomeric purity include at least 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

In certain embodiments, the enantiomer present in abundance (i.e., present in a greater quantity than the other enantiomer) is the (−)-enantiomer. In certain embodiments, the enantiomer present in abundance is the (+)-enantiomer. In certain embodiments, the enantiomer present in abundance is the (R)-enantiomer. In certain embodiments, the enantiomer present in abundance is the (S)-enantiomer.

In another aspect, the invention provides a deuterium-enriched compound of formula Ia or Ib or a pharmaceutically acceptable salt form thereof:

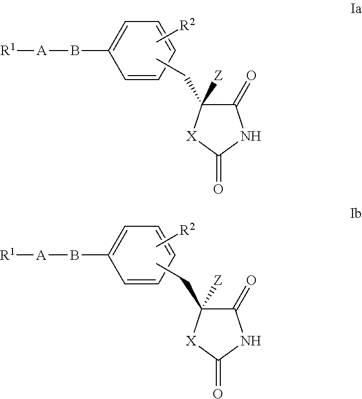

wherein Z, R¹, R², A, B, and X are as defined above for formula I.

In another aspect, the invention provides a deuterium-enriched compound of formula Ia or Ib or a pharmaceutically acceptable salt form thereof:

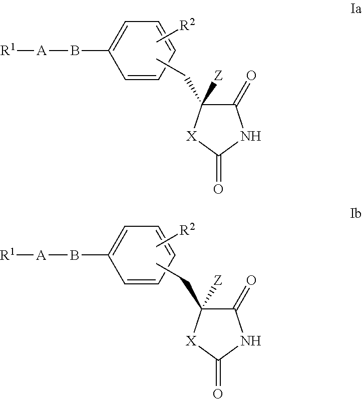

wherein Z, R¹, R², A, B, and X are as defined above for formula I, and the compound of formula Ia or Ib has an enantiomeric excess, with respect to the C—Z carbon, of at least 5%.

In another aspect, the invention provides a deuterium-enriched compound of formula II or a stereoisomer or pharmaceutically acceptable salt form thereof:

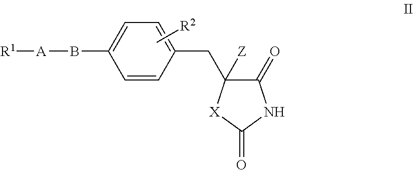

wherein Z, R¹, R², A, B, and X are as defined above for formula I.

In another aspect, the invention provides a deuterium-enriched compound of formula IIa or IIb or pharmaceutically acceptable salt form thereof:

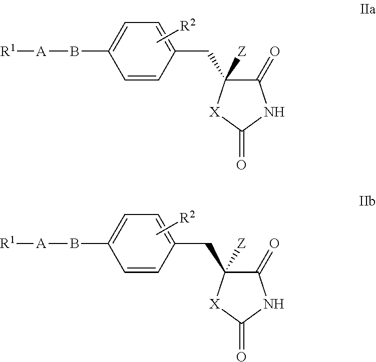

wherein Z, R¹, R², A, B, and X are as defined above for formula I.

In another aspect, the invention provides a deuterium-enriched compound of formula IIa or IIb or pharmaceutically acceptable salt form thereof:

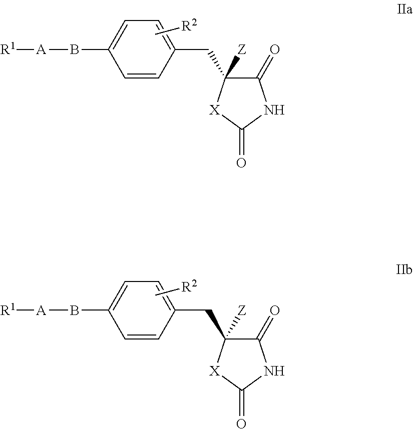

wherein Z, R¹, R², A, B, and X are as defined above for formula I and the compound of formula IIa or IIb has an enantiomeric excess, with respect to the C—Z carbon, of at least 5%.

In another aspect, the invention provides a deuterium-enriched compound of formula III or a stereoisomer or pharmaceutically acceptable salt form thereof:

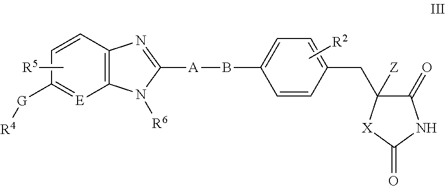

wherein Z, R², R⁴, R⁵, R⁶, A, B, E, G, and X are as defined above for formula I.

In another aspect, the invention provides a deuterium-enriched compound of formula IIIa or IIIb or pharmaceutically acceptable salt form thereof:

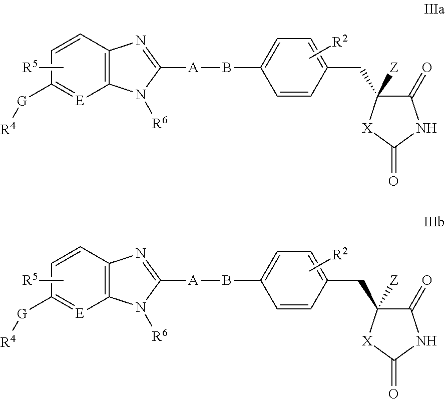

wherein Z, R², R⁴, R⁵, R⁶, A, B, E, G, and X are as defined above for formula I.

In another aspect, the invention provides a deuterium-enriched compound of formula IIIa or IIIb or pharmaceutically acceptable salt form thereof:

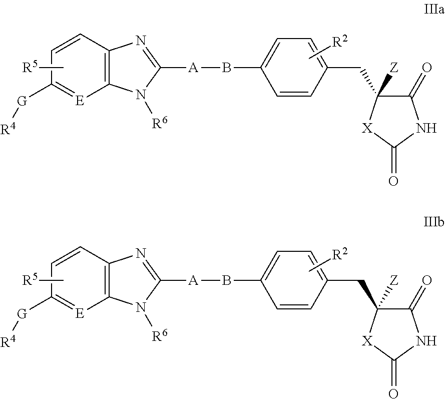

wherein Z, R², R⁴, R⁵, R⁶, A, B, E, G, and X are as defined above for formula I and the compound of formula IIIa or IIIb has an enantiomeric excess, with respect to the C—Z carbon, of at least 5%.

In another aspect, the invention provides a deuterium-enriched compound of formula IV or a stereoisomer or pharmaceutically acceptable salt form thereof:

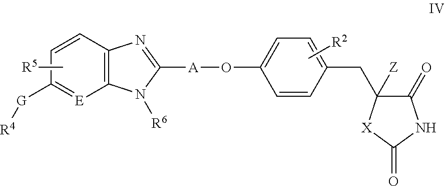

wherein Z, R², R⁴, R⁵, R⁶, A, E, G, and X are as defined above for formula I.

In another aspect, the invention provides a deuterium-enriched compound of formula IVa or IVb or pharmaceutically acceptable salt form thereof:

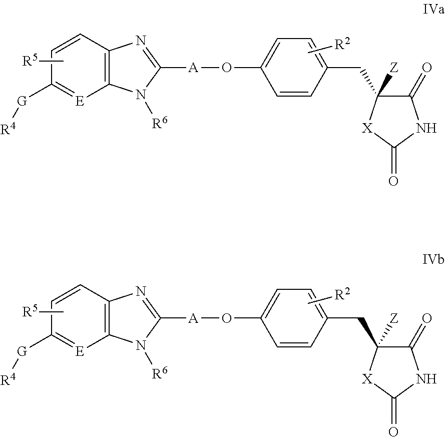

wherein Z, R², R⁴, R⁵, R⁶, A, E, G, and X are as defined above for formula I.

In another aspect, the invention provides a deuterium-enriched compound of formula IVa or IVb or pharmaceutically acceptable salt form thereof:

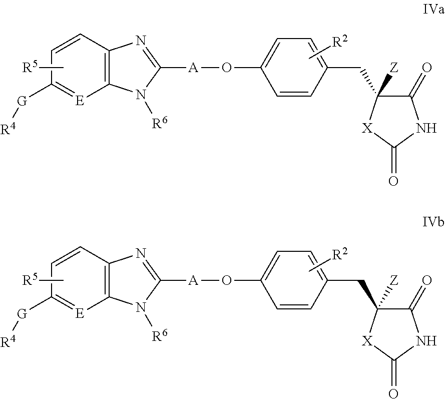

wherein Z, R², R⁴, R⁵, R⁶, A, E, G, and X are as defined above for formula I, and the compound of formula IVa or IVb has an enantiomeric excess, with respect to the C—Z carbon, of at least 5%.

In another aspect, the invention provides a deuterium-enriched compound of formula V or a stereoisomer or pharmaceutically acceptable salt form thereof:

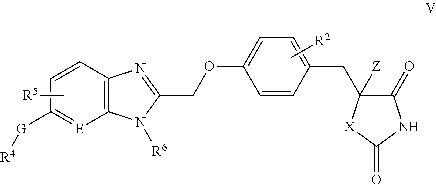

wherein Z, R², R⁴, R⁵, R⁶, E, G, and X are as defined above for formula I.

In another aspect, the invention provides a deuterium-enriched compound of formula Va or Vb or pharmaceutically acceptable salt form thereof:

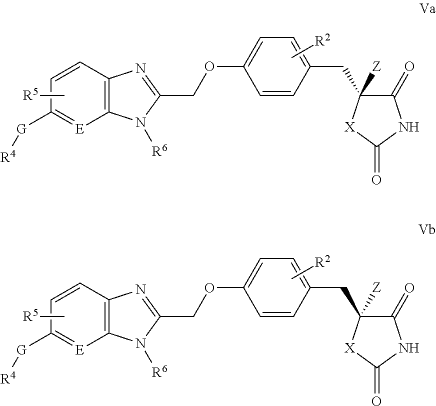

wherein Z, R², R⁴, R⁵, R⁶, E, G, and X are as defined above for formula I.

In another aspect, the invention provides a deuterium-enriched compound of formula Va or Vb or pharmaceutically acceptable salt form thereof:

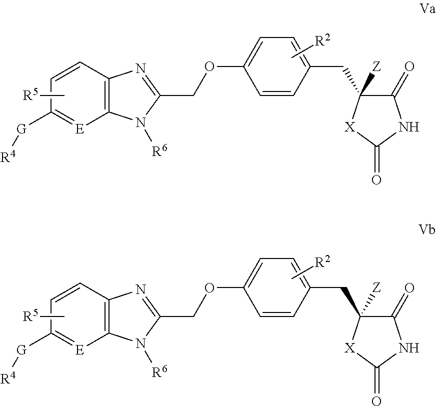

wherein Z, R², R⁴, R⁵, R⁶, E, G, and X are as defined above for formula I and the compound of formula Va or Vb has an enantiomeric excess, with respect to the C—Z carbon, of at least 5%.

In another aspect, the invention provides a deuterium-enriched compound of formula VI or a stereoisomer or pharmaceutically acceptable salt form thereof:

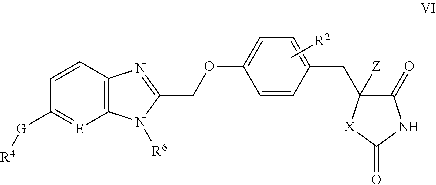

wherein Z, R², R⁴, R⁶, E, G, and X are as defined above for formula I.

In another aspect, the invention provides a deuterium-enriched compound of formula VIa or VIb or pharmaceutically acceptable salt form thereof:

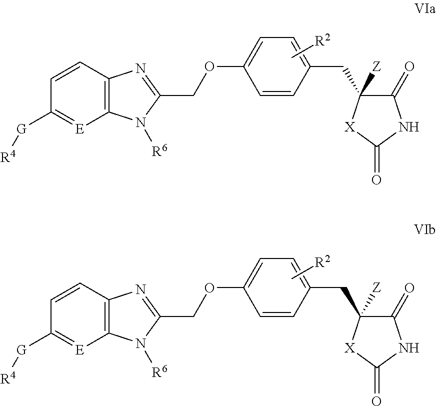

wherein Z, R², R⁴, R⁶, E, G, and X are as defined above for formula I.

In another aspect, the invention provides a deuterium-enriched compound of formula VIa or VIb or pharmaceutically acceptable salt form thereof:

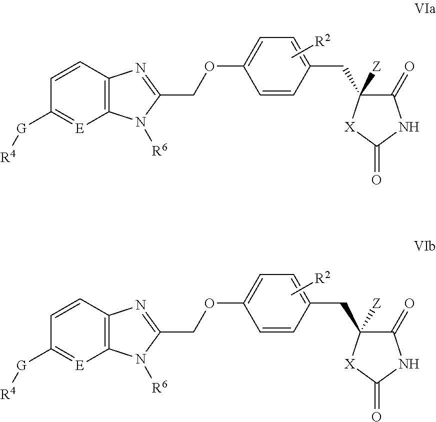

wherein Z, R², R⁴, R⁶, E, G, and X are as defined above for formula I and the compound of formula VIa or VIb has an enantiomeric excess, with respect to the C—Z carbon, of at least 5%.

In another aspect, the invention provides a deuterium-enriched compound of formula VII or a stereoisomer or pharmaceutically acceptable salt form thereof:

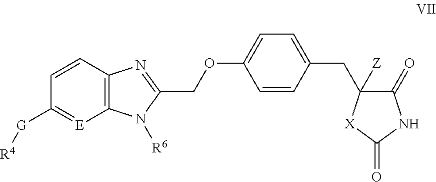

wherein Z, R⁴, R⁶, E, G, and X are as defined above for formula I.

In another aspect, the invention provides a deuterium-enriched compound of formula VIIa or VIIb or pharmaceutically acceptable salt form thereof:

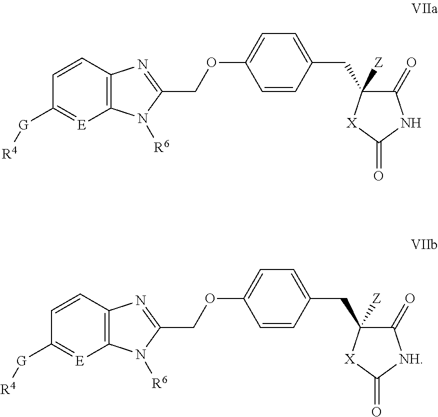

wherein Z, R⁴, R⁶, E, G, and X are as defined above for formula I.

In another aspect, the invention provides a deuterium-enriched compound of formula VIIa or VIIb or pharmaceutically acceptable salt form thereof:

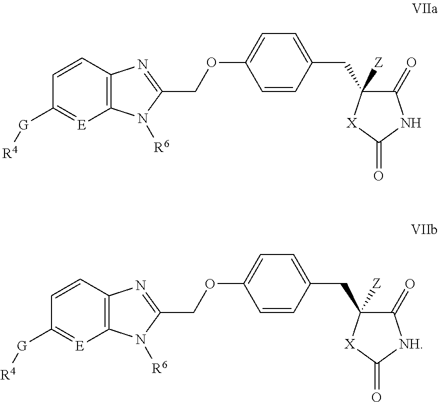

wherein Z, R⁴, R⁶, E, G, and X are as defined above for formula I and the compound of formula VIIa or VIIb has an enantiomeric excess, with respect to the C—Z carbon, of at least 5%.

In another aspect, the invention provides a deuterium-enriched compound of formula VIII or a stereoisomer or pharmaceutically acceptable salt form thereof:

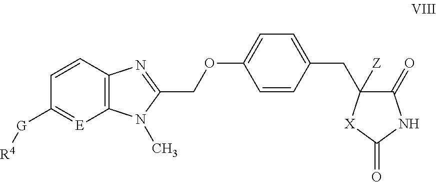

wherein Z, R⁴, E, G, and X are as defined above for formula I.

In another aspect, the invention provides a deuterium-enriched compound of formula VIIIa or VIIIb or pharmaceutically acceptable salt form thereof:

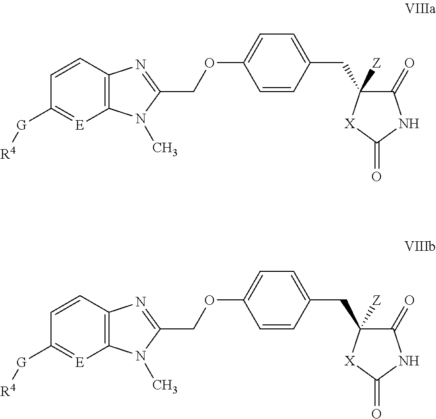

wherein Z, R⁴, E, G, and X are as defined above for formula I.

In another aspect, the invention provides a deuterium-enriched compound of formula VIIIa or VIIIb or pharmaceutically acceptable salt form thereof:

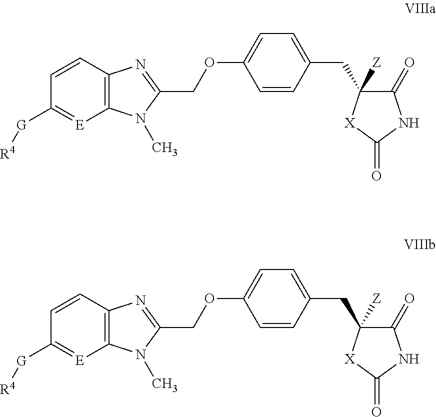

wherein Z, R⁴, E, G, and X are as defined above for formula I, and the compound of formula VIIIa or VIIIb has an enantiomeric excess, with respect to the C—Z carbon, of at least 5%.

In another aspect, the invention provides a deuterium-enriched compound of formula IX or a stereoisomer or pharmaceutically acceptable salt form thereof:

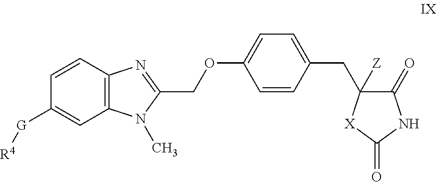

wherein Z, R⁴, G, and X are as defined above for formula I.

In another aspect, the invention provides a deuterium-enriched compound of formula IXa or IXb or pharmaceutically acceptable salt form thereof:

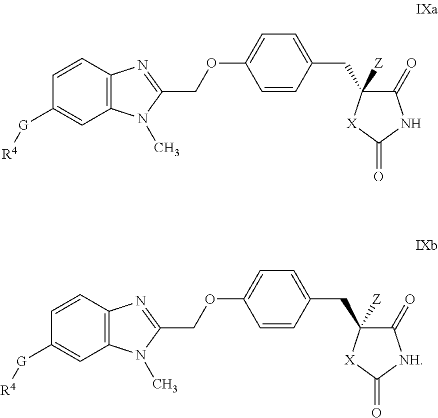

wherein Z, R⁴, G, and X are as defined above for formula I.

In another aspect, the invention provides a deuterium-enriched compound of formula IXa or IXb or pharmaceutically acceptable salt form thereof:

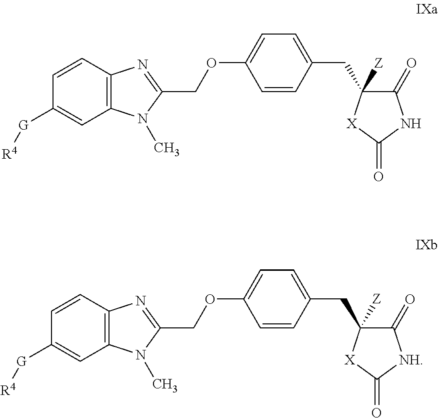

wherein Z, R⁴, G, and X are as defined above for formula I, and the compound of formula IXa or IXb has an enantiomeric excess, with respect to the C—Z carbon, of at least 5%.

In another aspect, the invention provides a deuterium-enriched compound of formula X or a stereoisomer or pharmaceutically acceptable salt form thereof:

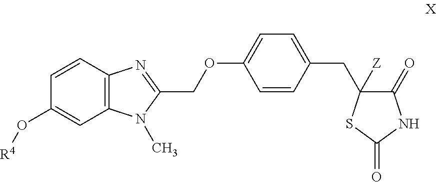

wherein Z and R⁴ are as defined above for formula I.

In another aspect, the invention provides a deuterium-enriched compound of formula Xa or Xb or pharmaceutically acceptable salt form thereof:

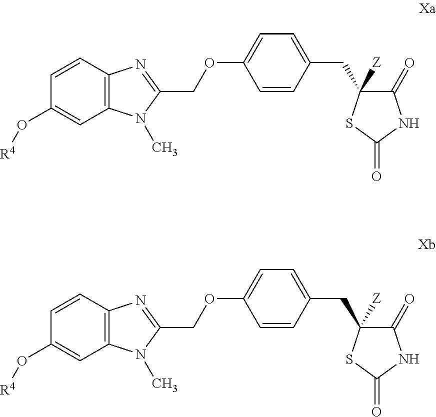

wherein Z and R⁴ are as defined above for formula I.

In another aspect, the invention provides a deuterium-enriched compound of formula Xa or Xb or pharmaceutically acceptable salt form thereof:

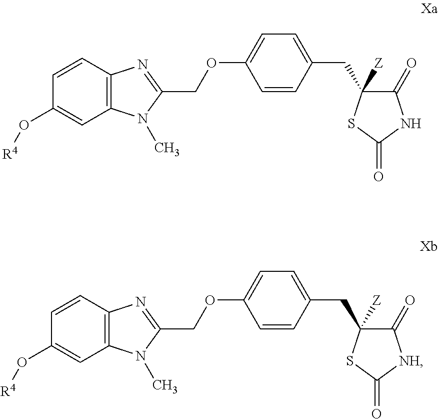

and the compound of formula Xa or Xb has an enantiomeric excess, with respect to the C—Z carbon, of at least 5%.

In another aspect, the invention provides a deuterium-enriched compound of formula XI or a stereoisomer or pharmaceutically acceptable salt form thereof:

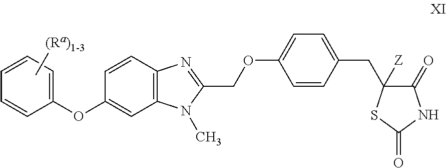

wherein Z and R^{a} are as defined above for formula I.

In another aspect, the invention provides a deuterium-enriched compound of formula XIa or XIb or pharmaceutically acceptable salt form thereof:

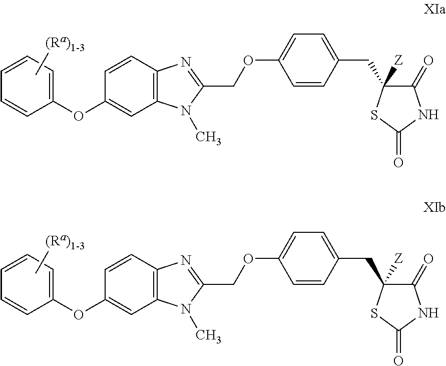

wherein Z and R^{a} are as defined above for formula I.

In another aspect, the invention provides a deuterium-enriched compound of formula XIa or XIb or pharmaceutically acceptable salt form thereof:

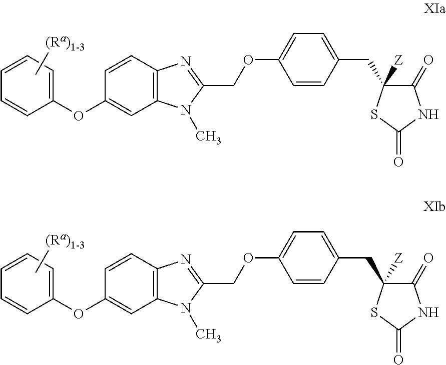

wherein Z and R^{a} are as defined above for formula I, and the compound of formula XIa or XIb has an enantiomeric excess, with respect to the C—Z carbon, of at least 5%.

In another aspect, the invention provides a deuterium-enriched compound of formula XII or a stereoisomer or pharmaceutically acceptable salt form thereof:

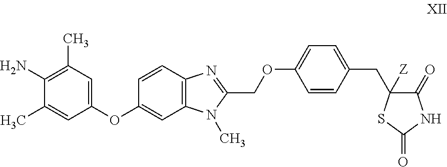

wherein Z is as defined above for formula I. In certain embodiments, the deuterium-enriched compound is a compound of formula XII or a stereoisomer thereof.

In another aspect, the invention provides a deuterium-enriched compound of formula XIIa or XIIb or pharmaceutically acceptable salt form thereof:

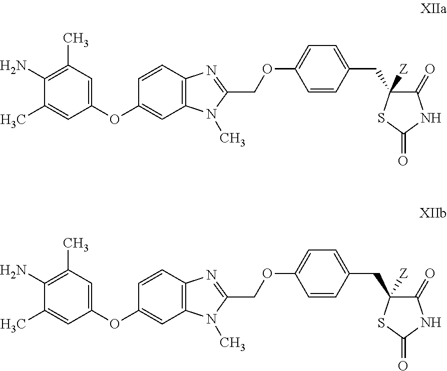

wherein Z is as defined above for formula I. In certain embodiments, the deuterium-enriched compound is a compound of formula XIIa or a pharmaceutically acceptable salt form thereof, such as where the abundance of deuterium in Z is at least 50%. In certain other embodiments, the deuterium-enriched compound is a compound of formula XIIa. In yet other embodiments, the deuterium-enriched compound is a compound of formula XIIb or a pharmaceutically acceptable salt form thereof, such as where the abundance of deuterium in Z is at least 50%. In yet other embodiments, the deuterium-enriched compound is a compound of formula XIIb.

In another aspect, the invention provides a deuterium-enriched compound of formula XIIa or XIIb or pharmaceutically acceptable salt form thereof:

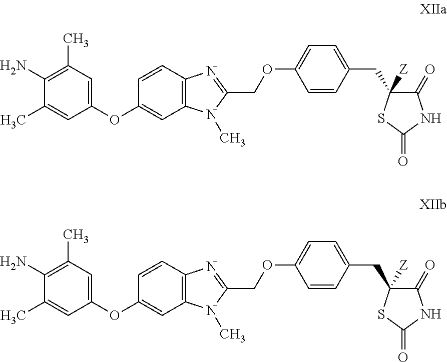

wherein Z is as defined above for formula I, and the compound of formula XIIa or XIIb has an enantiomeric excess, with respect to the C—Z carbon, of at least 5%. In certain embodiments, the deuterium-enriched compound is a compound of formula XIIa or a pharmaceutically acceptable salt form thereof, such as where the abundance of deuterium in Z is at least 50%. In certain other embodiments, the deuterium-enriched compound is a compound of formula XIIa. In yet other embodiments, the deuterium-enriched compound is a compound of formula XIIb or a pharmaceutically acceptable salt form thereof, such as where the abundance of deuterium in Z is at least 50%. In yet other embodiments, the deuterium-enriched compound is a compound of formula XIIb.

In another aspect, the invention provides a deuterium-enriched compound of formula XII₁ or a stereoisomer or pharmaceutically acceptable salt form thereof:

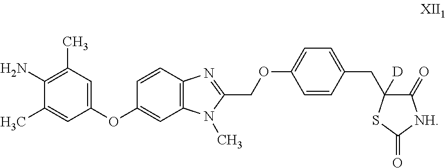 [SMILES: [2H]C1(Cc2ccc(OCc3nc4ccc(Oc5cc(C)c(N)c(C)c5)cc4n3C)cc2)SC(=O)NC1=O]

In another aspect, the invention provides a deuterium-enriched compound of formula XIIa₁ or XIIb₁ or pharmaceutically acceptable salt form thereof:

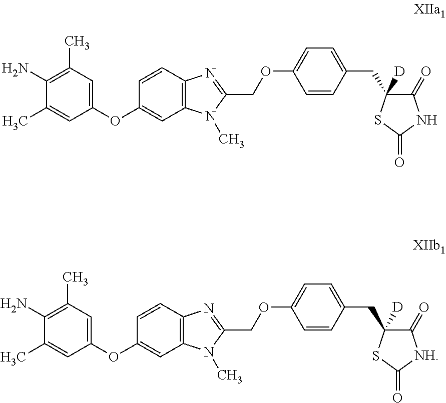 [SMILES: [2H][C@]1(Cc2ccc(OCc3nc4ccc(Oc5cc(C)c(N)c(C)c5)cc4n3C)cc2)SC(=O)NC1=O ; [2H][C@@]1(Cc2ccc(OCc3nc4ccc(Oc5cc(C)c(N)c(C)c5)cc4n3C)cc2)SC(=O)NC1=O]

In another aspect, the invention provides a deuterium-enriched compound of formula XIIa₁ or XIIb₁ or pharmaceutically acceptable salt form thereof:

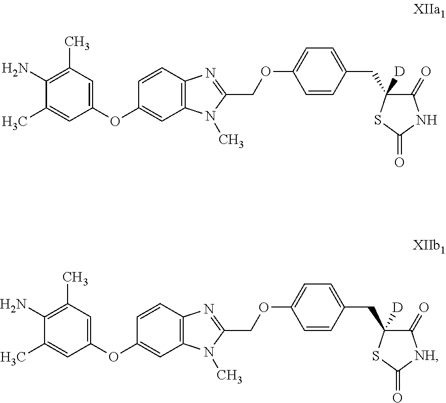 [SMILES: [2H][C@]1(Cc2ccc(OCc3nc4ccc(Oc5cc(C)c(N)c(C)c5)cc4n3C)cc2)SC(=O)NC1=O ; [2H][C@@]1(Cc2ccc(OCc3nc4ccc(Oc5cc(C)c(N)c(C)c5)cc4n3C)cc2)SC(=O)NC1=O]

wherein the compound of formula XIIa₁ or XIIb₁ has an enantiomeric excess, with respect to the C-D carbon, of at least 5%.

In another aspect, the invention provides a deuterium-enriched compound, wherein: (1) R¹ is:

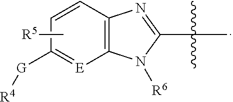

In another aspect, the invention provides a deuterium-enriched compound, wherein: (2) R² and R⁵ are the same or different and are selected from: H, D, halogen, hydroxyl, C₁₋₆ alkyl, halo-C₁₋₆ alky, C₁₋₆ alkoxy, C₁₋₆ alkylthio, and NH₂ optionally substituted with R^{c}.

In another aspect, the invention provides a deuterium-enriched compound, wherein: (3) R² and R⁵ are the same or different and are selected from H, D, F, Cl, OH, CH₃, CH₃CH₂, CF₃, CH₃O, CH₃S, and NH₂.

In another aspect, the invention provides a deuterium-enriched compound, wherein: (4) R² and R⁵ each are H or D.

In another aspect, the invention provides a deuterium-enriched compound, wherein: (5) X is S.

In another aspect, the invention provides a deuterium-enriched compound, wherein: (6) X is O.

In another aspect, the invention provides a deuterium-enriched compound, wherein: (7) R⁴ is pyridyl optionally substituted with 1 substituent selected from: halo, hydroxyl, C₁₋₆ alkyl group, halo-C₁₋₆ alkyl, C₁₋₆ alkoxy group, C₁₋₆ alkylthio, NH₂ optionally substituted with 1-2 R^{c}, and NO₂.

In another aspect, the invention provides a deuterium-enriched compound, wherein: (8) R⁴ represents pyridyl optionally substituted with 1 substituent selected from: F, Cl, OH, CH₃, CH₃CH₂, (CH₃)₃C, CF₃, CH₃O, CH₃S, NH₂, CH₃NH, (CH₃)₂N, and NO₂.

In another aspect, the invention provides a deuterium-enriched compound, wherein: (9) R⁴ is pyridyl.

In another aspect, the invention provides a deuterium-enriched compound, wherein: (10) R⁴ is phenyl substituted with 1-2 substituents selected from C₃₋₁₀ cycloalkyl optionally substituted with 1-3 R^{b}, C₆₋₁₀ aryl optionally substituted with 1-3 R^{b}, C₇₋₁₆ aralkyl optionally substituted with 1-3 R^{b}, 4-7 membered saturated nitrogen-containing heterocyclic group, 5-6 membered aromatic nitrogen-containing heterocyclic group.

In another aspect, the invention provides a deuterium-enriched compound, wherein: (11) R⁴ is phenyl substituted with one substituent selected from: C₃₋₁₀ cycloalkyl optionally substituted with 1 R^{b}, C₆₋₁₀ aryl optionally substituted with 1 R^{b}, C₇₋₁₆ aralkyl optionally substituted with 1 R^{b}, 4-7 membered saturated nitrogen-containing heterocyclic group, 5-6 membered aromatic nitrogen-containing heterocyclic group.

In another aspect, the invention provides a deuterium-enriched compound, wherein: (12) R⁴ is phenyl substituted with one substituent selected from: C₃₋₁₀ cycloalkyl optionally substituted with 1 R^{b}, C₆₋₁₀ aryl optionally substituted with 1 R^{b}, C₇₋₁₆ aralkyl optionally substituted with 1 R^{b}, 4-7 membered saturated nitrogen-containing heterocyclic group, 5-6 membered aromatic nitrogen-containing heterocyclic group, wherein R^{b}, for the purposes of R⁴, is selected from: halo, hydroxyl, C₁₋₆ alkyl, halo-C₁₋₆ alkyl, C₁₋₆ alkoxy, and NH₂ optionally substituted with 1-2 R^{c}.

In another aspect, the invention provides a deuterium-enriched compound, wherein: (13) R⁴ is phenyl group substituted with one substituent selected from: phenyl optionally substituted with 1 R^{b}, benzyl optionally substituted with 1 R^{b}, adamantyl, pyrrolidinyl, morpholinyl, piperidinyl, imidazolyl, tetrazolyl, and pyridinyl, wherein R^{b}, for the purposes of R⁴, is selected from: halo, hydroxyl, C₁₋₆ alkyl, halo-C₁₋₆ alkyl, C₁₋₆ alkoxy, and NH₂ optionally substituted with 1-2 R^{c}.

In another aspect, the invention provides a deuterium-enriched compound, wherein: (14) R⁴ is phenyl substituted with one substituent selected from: phenyl optionally substituted with 1 R^{b}, benzyl optionally substituted with 1 R^{b}, adamantyl, pyrrolidinyl, morpholinyl, piperidinyl, imidazolyl, tetrazolyl, and pyridinyl, wherein R^{b}, for the purposes of R⁴, is selected from: F, Cl, OH, CH₃, CH₃CH₂, (CH₃)₃C, CF₃, CH₃O, CH₃S, NH₂, CH₃NH, (CH₃)₂N, and NO₂.

In another aspect, the invention provides a deuterium-enriched compound, wherein: (15) R⁴ is selected from: 4-biphenylyl, 4-benzylphenyl, 4′-hydroxybiphenylyl, (pyrrolidin-1-yl)phenyl, (morpholin-4-yl)phenyl, (piperidin-1-yl)phenyl, (pyridin-2-yl)phenyl, (pyridin-3-yl)phenyl, and 4-(1-adamantyl)phenyl group.

In another aspect, the invention provides a deuterium-enriched compound, wherein: (16) R⁴ is phenyl substituted with one acylamino group, wherein the amino moiety is optionally substituted with 1 R^{c} and the phenyl is optionally further substituted with 1-3 substituents selected from: halo, hydroxyl, C₁₋₆ alkyl, halo-C₁₋₆ alkyl, C₁₋₆ alkoxy, and C₁₋₆ alkylthio.

In another aspect, the invention provides a deuterium-enriched compound, wherein: (17) R⁴ is phenyl substituted with one acylamino group, wherein the amino moiety is optionally substituted with 1 R^{c} and the phenyl is optionally further substituted with 1-3 substituents selected from: halo and C₁₋₆ alkyl.

In another aspect, the invention provides a deuterium-enriched compound, wherein: (18) R⁴ is phenyl substituted with one acylamino group, wherein the amino moiety is optionally substituted with C₁₋₆ alkyl group or C₇₋₁₂ aralkyl optionally substituted with 1-2 R^{b}, and the phenyl is optionally further substituted with 1-3 C₁₋₆ alkyl.

In another aspect, the invention provides a deuterium-enriched compound, wherein: (19) R⁴ is phenyl substituted with one substituent selected from: C₇₋₁₁ aromatic acylamino optionally substituted with 1-2 R^{d}, C₄₋₁₁ cycloalkylcarbonylamino optionally substituted with 1-2 R^{d}, and 5-6-membered aromatic nitrogen-containing heterocyclic carbonylamino optionally substituted with 1-2 R^{d}.

In another aspect, the invention provides a deuterium-enriched compound, wherein: (20) R⁴ is phenyl substituted with 1 substituent selected from: benzoylamino, 3-chlorobenzoylamino, 2,4-difluorobenzoylamino, 4-hydroxy-3,5-di-t-butylbenzoylamino, naphthoylamino, cyclopentanoylamino, cyclohexanoylamino, nicotinoylamino, isonicotinoylamino, N-acetyl-N-hexylamino, and adamantylcarbonylamino.

In another aspect, the invention provides a deuterium-enriched compound, wherein: (21) R⁴ is phenyl substituted with 1 substituent selected from: NH₂ optionally substituted with 1-2 R^{c}, NO₂, and —CN, and the phenyl is optionally further substituted with 1-3 substituents selected from: halo, hydroxyl, C₁₋₆ alkyl, haloC₁₋₆ alkyl, C₁₋₆ alkoxy, and C₁₋₆ alkylthio, wherein R^{c} for the purposes of R⁴ is selected from: C₁₋₁₀ alkyl optionally substituted with 1-2 R^{d}, C₆₋₁₀ aryl optionally substituted with 1-2 R^{d}, and C₇₋₁₆ aralkyl optionally substituted with 1-2 R^{d}.

In another aspect, the invention provides a deuterium-enriched compound, wherein: (22) R⁴ is phenyl substituted with 1 substituent selected from: NH₂, (C₁₋₁₀ alkyl)NH, (C₁₋₁₀ alkyl)₂N, and —CN and the phenyl is optionally substituted with 1-2 C₁₋₆ alkyl.

In another aspect, the invention provides a deuterium-enriched compound, wherein: (23) R⁴ is selected from: 4-aminophenyl, 4-amino-3,5-dimethylphenyl, 4-amino-3,5-di-t-butylphenyl, 3- or 4-dimethylaminophenyl, and 4-cyanophenyl group.

In another aspect, the invention provides a deuterium-enriched compound, wherein: (24) R⁴ is a phenyl substituted with 1 substituent selected from: C₆₋₁₀ aryloxy optionally substituted with 1-3 R^{b}, C₇₋₁₆ aralkyloxy aryloxy optionally substituted with 1-3 R^{b}, and C₆₋₁₀ arylthio aryloxy optionally substituted with 1-3 R^{b}, and the phenyl is optionally further substituted with 1-3 substituents selected from: halo, hydroxyl, C₁₋₆ alkyl, haloC₁₋₆ alkyl, C₁₋₆ alkoxy, and C₁₋₆ alkylthio.

In another aspect, the invention provides a deuterium-enriched compound, wherein: (25) R⁴ is phenyl substituted with C₆₋₁₀ aryloxy optionally substituted with 1-3 R^{b}, and the phenyl is optionally further substituted with 1-2 C₁₋₆ alkyl groups.

In another aspect, the invention provides a deuterium-enriched compound, wherein: (26) R⁴ is phenyl substituted with C₆₋₁₀ aryloxy optionally substituted with 1 R^{a}.

In another aspect, the invention provides a deuterium-enriched compound, wherein: (27) R⁴ is 4-phenoxyphenyl.

In another aspect, the invention provides a deuterium-enriched compound, wherein: (28) R⁴ is phenyl substituted with 1-5 substituents selected from: halo, hydroxyl, C₁₋₆ alkyl, halo-C₁₋₆ alkyl, C₁₋₆ alkoxy, C₁₋₆ alkylthio, and C₁₋₇ aliphatic acyloxy.

In another aspect, the invention provides a deuterium-enriched compound, wherein: (29) R⁴ is phenyl substituted with 1 group selected from: halo, hydroxyl, C₁₋₆ alkyl, halo-C₁₋₆ alkyl, C₁₋₆ alkoxy, C₁₋₆ alkylthio, and C₁₋₇ aliphatic acyloxy, and the phenyl is optionally further substituted with 1-4 substituents selected from: halo, C₁₋₆ alkyl, and halo-C₁₋₆ alkyl.

In another aspect, the invention provides a deuterium-enriched compound, wherein: (30) R⁴ is phenyl substituted with 1 group selected from: C₁₋₆ alkyl group, halo-C₁₋₆ alkyl, C₁₋₆ alkoxy, and C₁₋₆ alkylthio group, or with 1-5 halo.

In another aspect, the invention provides a deuterium-enriched compound, wherein: (31) R⁴ is phenyl group substituted with 1 group selected from: halo-C₁₋₂ alkyl, C₁₋₂ alkoxy, and C₁₋₂ alkylthio group or with 1-5 fluorine or chlorine.

In another aspect, the invention provides a deuterium-enriched compound, wherein: (32) R⁴ is selected from: 4-trifluoromethylphenyl, 4-methylthiophenyl, 4-methoxyphenyl, and pentafluorophenyl group.

In another aspect, the invention provides a deuterium-enriched compound, wherein: (33) R⁴ is phenyl group substituted with 1 group selected from: hydroxyl and C₁₋₇ aliphatic acyloxy group, and that phenyl is optionally further substituted with 1-3 substituents selected from: halo and C₁₋₆ alkyl.

In another aspect, the invention provides a deuterium-enriched compound, wherein: (34) R⁴ is group substituted with 1 hydroxyl group, and the phenyl is optionally further substituted with 1-3 substituents selected from: halo and C₁₋₆ alkyl.

In another aspect, the invention provides a deuterium-enriched compound, wherein: (35) R⁴ is phenyl substituted with one hydroxyl group, and the phenyl is optionally further substituted with 1-3 substituents selected from: F, Cl, methyl, and t-butyl.

In another aspect, the invention provides a deuterium-enriched compound, wherein: (36) R⁴ is selected from: 4-hydroxyphenyl, 4-hydroxy-3,5-dimethylphenyl, 4-hydroxy-3,5-di-t-butylphenyl, 4-hydroxy-2,3,5-trimethylphenyl, and 2-chloro-4-hydroxy-3,5-dimethylphenyl group.

In another aspect, the invention provides a deuterium-enriched compound, wherein: (37) R⁶ is selected from: H, D, C₁₋₆ alkyl, phenyl, and benzyl, wherein the phenyl or benzyl are optionally substituted with 1-3 substituents selected from: halo, hydroxyl, C₁₋₆ alkyl, and halo-C₁₋₆ alkyl.

In another aspect, the invention provides a deuterium-enriched compound, wherein: (38) R⁶ is selected from: H, D, C₁₋₄ alkyl, phenyl, and benzyl, wherein the phenyl or benzyl are optionally substituted with 1 substituent selected from: F, Cl, hydroxyl, methyl, and ethyl.

In another aspect, the invention provides a deuterium-enriched compound, wherein: (39) R⁶ is selected from H, D, and C₁₋₄ alkyl.

In another aspect, the invention provides a deuterium-enriched compound, wherein: (40) R⁶ is C₁₋₂ alkyl.

In another aspect, the invention provides a deuterium-enriched compound, wherein: (41) R⁶ is methyl.

In another aspect, the invention provides a deuterium-enriched compound, wherein: (42) A is C₁₋₄ alkylene.

In another aspect, the invention provides a deuterium-enriched compound, wherein: (43) wherein A is C₁₋₂ alkylene.

In another aspect, the invention provides a deuterium-enriched compound, wherein: (44) A is methylene.

In another aspect, the invention provides a deuterium-enriched compound, wherein: (45) B is oxygen.

In another aspect, the invention provides a deuterium-enriched compound, wherein: (46) G is oxygen.

In another aspect, the invention provides a deuterium-enriched compound, wherein: (47) G is sulfur.

In another aspect, the invention provides a deuterium-enriched compound, wherein: (48) E is CH.

In another aspect, the invention provides a deuterium-enriched compound, wherein: (49) E is nitrogen.

In another aspect, the invention provides a deuterium-enriched compound, wherein: the substituents are any combinations of two to nine groups selected from aspects (1), (2)-(4), (5)-(6), (7)-(36), (37)-(41), (42)-(44), (45), (46)-(47) and (48)-(49).

In another aspect, the invention provides a deuterium-enriched compound of Formula XIII or a stereoisomer or pharmaceutically acceptable salt thereof:

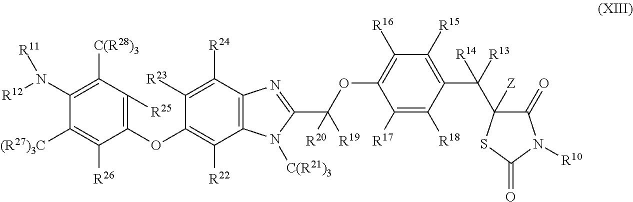

wherein R¹⁰ through R²⁸ are independently hydrogen or D; and Z is H or D, provided that the abundance of deuterium in Z is at least 50%.

In another aspect, the invention provides a deuterium-enriched compound of Formula XIIIa or a pharmaceutically acceptable salt thereof:

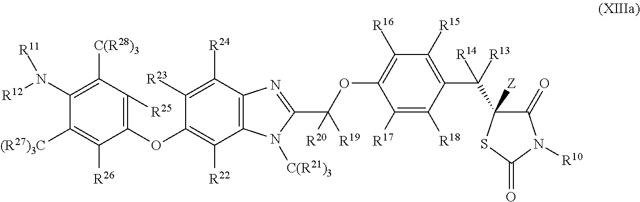

wherein R¹⁰ through R²⁸ are independently hydrogen or D; and Z is H or D, provided that the abundance of deuterium in Z is at least 50%, and the compound has an enantiomeric excess, with respect to the C—Z carbon, of at least 70%.

In another aspect, the invention provides a deuterium-enriched compound of Formula XIIIb or a pharmaceutically acceptable salt thereof:

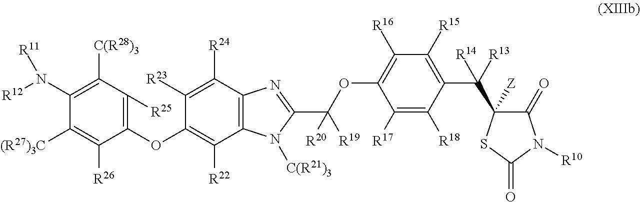

wherein R¹⁰ through R²⁸ are independently hydrogen or D; and Z is H or D, provided that the abundance of deuterium in Z is at least 50%, and the compound has an enantiomeric excess, with respect to the C—Z carbon, of at least 70%.

In another aspect, the invention provides a deuterium-enriched compound of Formula XIV or a stereoisomer or pharmaceutically acceptable salt thereof:

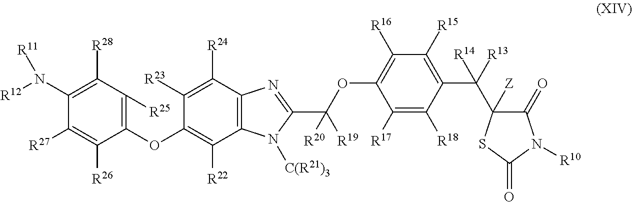

wherein R¹⁰ through R²⁸ are independently hydrogen or D; and Z is H or D, provided that the abundance of deuterium in Z is at least 50%.

In another aspect, the invention provides a deuterium-enriched compound of Formula XIVa or a pharmaceutically acceptable salt thereof:

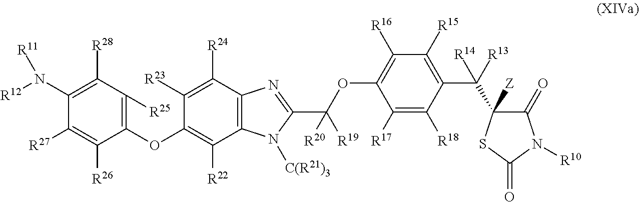

wherein R¹⁰ through R²⁸ are independently hydrogen or D; and Z is H or D, provided that the abundance of deuterium in Z is at least 50%, and the compound has an enantiomeric excess, with respect to the C—Z carbon, of at least 70%.

In another aspect, the invention provides a deuterium-enriched compound of Formula XIVb or a pharmaceutically acceptable salt thereof:

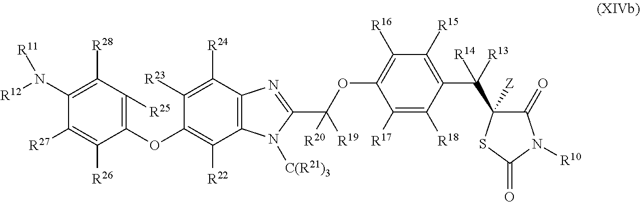

wherein R¹⁰ through R²⁸ are independently hydrogen or D; and Z is H or D, provided that the abundance of deuterium in Z is at least 50%, and the compound has an enantiomeric excess, with respect to the C—Z carbon, of at least 70%.

In another aspect, the invention provides a deuterium-enriched compound of formula XV or a stereoisomer or pharmaceutically acceptable salt form thereof:

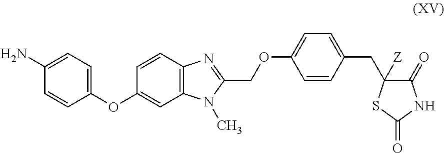

wherein Z is as defined above for formula I. In certain embodiments, the deuterium-enriched compound is a compound of formula XV or a stereoisomer thereof.

In another aspect, the invention provides a deuterium-enriched compound of formula XVa or XVb or pharmaceutically acceptable salt form thereof:

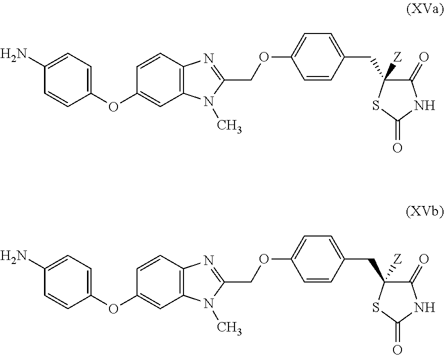

wherein Z is as defined above for formula I, and the compound of formula XVa or XVb has an enantiomeric excess of at least 5%. In certain embodiments, the deuterium-enriched compound is a compound of formula XVa or a pharmaceutically acceptable salt form thereof, such as where the abundance of deuterium in Z is at least 50%. In certain other embodiments, the deuterium-enriched compound is a compound of formula XVa. In yet other embodiments, the deuterium-enriched compound is a compound of formula XVb or a pharmaceutically acceptable salt form thereof, such as where the abundance of deuterium in Z is at least 50%. In yet other embodiments, the deuterium-enriched compound is a compound of formula XVb.

In another aspect, the invention provides a deuterium-enriched compound of formula XVI₁ or a stereoisomer or pharmaceutically acceptable salt form thereof:

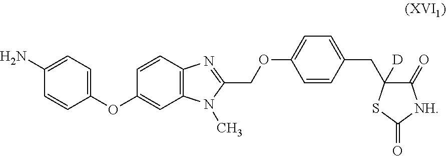 [SMILES: [2H]C1(Cc2ccc(OCc3nc4ccc(Oc5ccc(N)cc5)cc4n3C)cc2)SC(=O)NC1=O]

In another aspect, the invention provides a deuterium-enriched compound of formula XVIa₁ or XVIb₁ or pharmaceutically acceptable salt form thereof:

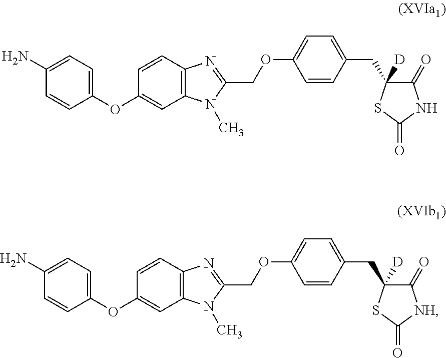 [SMILES: [2H][C@]1(Cc2ccc(OCc3nc4ccc(Oc5ccc(N)cc5)cc4n3C)cc2)SC(=O)NC1=O ; [2H][C@@]1(Cc2ccc(OCc3nc4ccc(Oc5ccc(N)cc5)cc4n3C)cc2)SC(=O)NC1=O]

wherein the compound of formula XVIa₁ or XVIb₁ has an enantiomeric excess of at least 5%. In certain embodiments, the compound of formula XVIa₁ or XVIb₁ has an enantiomeric excess of at least 80%, 85%, 90%, 95%, or 98%.

In another aspect, the invention provides a deuterium-enriched compound of Formula XVII or a stereoisomer or pharmaceutically acceptable salt thereof:

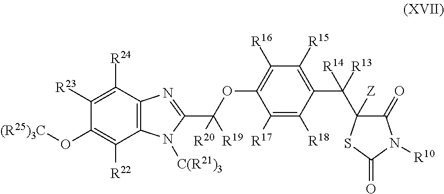

wherein each of R¹⁰ and R¹³ through R²⁵ are independently hydrogen or D; and Z is H or D, provided that the abundance of deuterium in Z is at least 30%.

In another aspect, the invention provides a deuterium-enriched compound of Formula XVIIa or a pharmaceutically acceptable salt thereof:

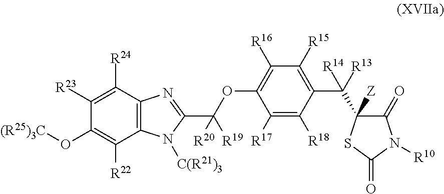

wherein each of R¹⁰ and R¹³ through R²⁵ are independently hydrogen or D; and Z is H or D, provided that the abundance of deuterium in Z is at least 30%, and the compound has an enantiomeric excess, with respect to the C—Z carbon, of at least 70%.

In another aspect, the invention provides a deuterium-enriched compound of Formula XVIIb or a pharmaceutically acceptable salt thereof:

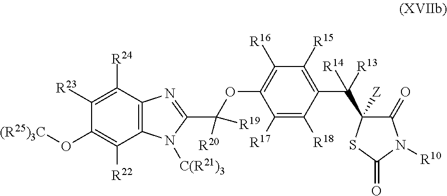

wherein each of R¹⁰ and R¹³ through R²⁵ are independently hydrogen or D; and Z is H or D, provided that the abundance of deuterium in Z is at least 30%, and the compound has an enantiomeric excess, with respect to the C—Z carbon, of at least 70%.

In another aspect, the invention provides a deuterium-enriched compound of formula XVIII or a stereoisomer or pharmaceutically acceptable salt form thereof:

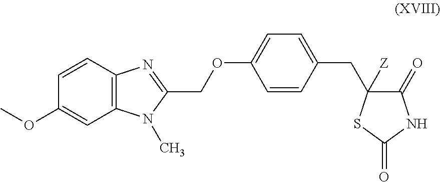

wherein Z is H or D, provided that the abundance of deuterium in Z is at least 30%. In certain embodiments, the deuterium-enriched compound is a compound of formula XVIII or a stereoisomer thereof.

In another aspect, the invention provides a deuterium-enriched compound of formula XVIIIa or XVIIIb or pharmaceutically acceptable salt form thereof:

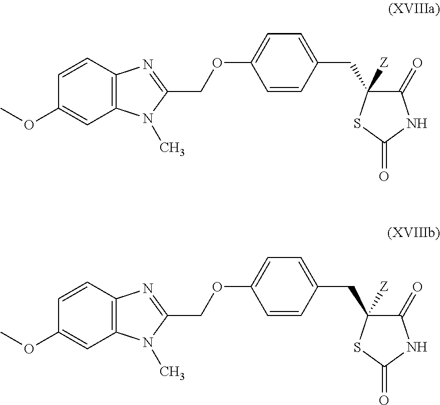

wherein Z is H or D, provided that the abundance of deuterium in Z is at least 30%, and the compound of formula XVIIIa or XVIIIb has an enantiomeric excess of at least 5%. In certain embodiments, the deuterium-enriched compound is a compound of formula XVIIIa or a pharmaceutically acceptable salt form thereof, such as where the abundance of deuterium in Z is at least 50%. In certain other embodiments, the deuterium-enriched compound is a compound of formula XVIIIa. In yet other embodiments, the deuterium-enriched compound is a compound of formula XVIIIb or a pharmaceutically acceptable salt form thereof, such as where the abundance of deuterium in Z is at least 50%. In yet other embodiments, the deuterium-enriched compound is a compound of formula XVIIIb.

In another aspect, the invention provides a deuterium-enriched compound of formula XIX₁ or a stereoisomer or pharmaceutically acceptable salt form thereof:

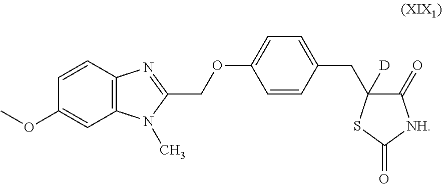 [SMILES: [2H]C1(Cc2ccc(OCc3nc4ccc(OC)cc4n3C)cc2)SC(=O)NC1=O]

In another aspect, the invention provides a deuterium-enriched compound of formula XIXa₁ or XIXb₁ or pharmaceutically acceptable salt form thereof:

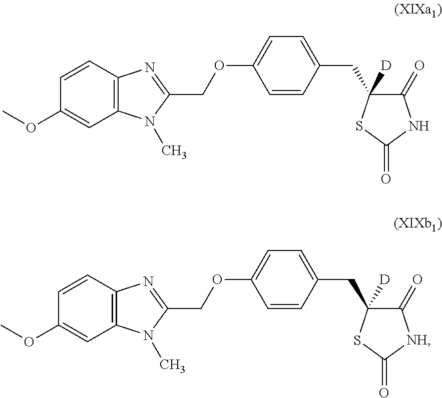 [SMILES: [2H][C@]1(Cc2ccc(OCc3nc4ccc(OC)cc4n3C)cc2)SC(=O)NC1=O ; [2H][C@@]1(Cc2ccc(OCc3nc4ccc(OC)cc4n3C)cc2)SC(=O)NC1=O]

wherein the compound of formula XIXa₁ or XIXb₁ has an enantiomeric excess of at least 5%. In certain embodiments, the compound of formula XIXa₁ or XIXb₁ has an enantiomeric excess of at least 80%, 85%, 90%, 95%, or 98%.

In another aspect, the invention provides a deuterium-enriched compound selected from:

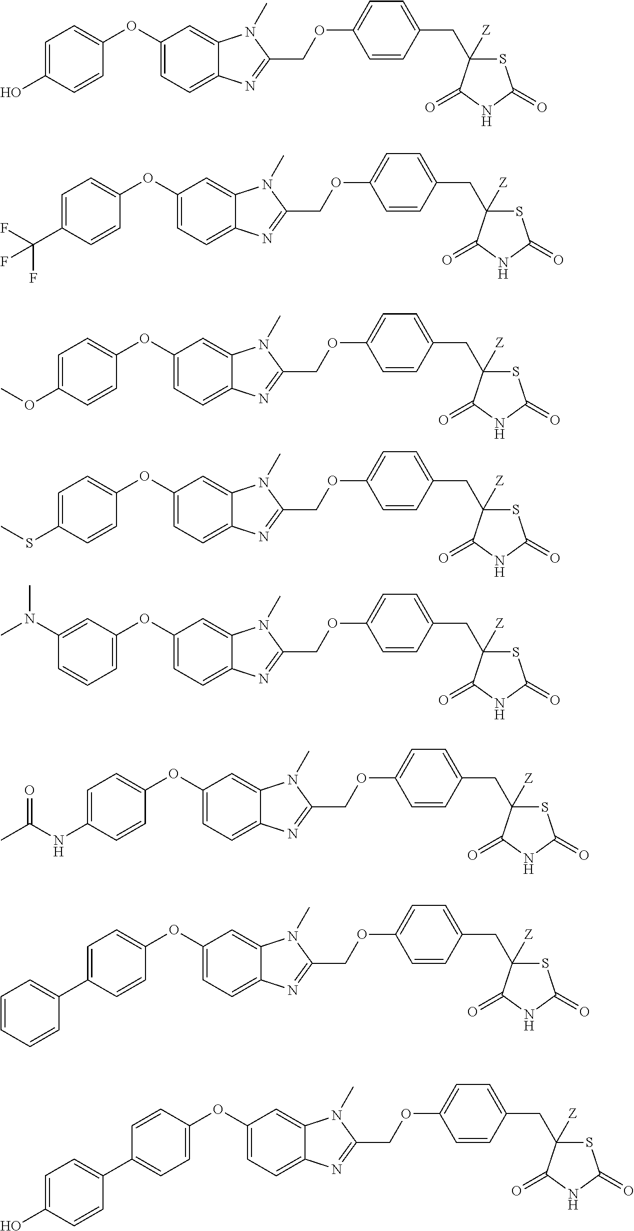

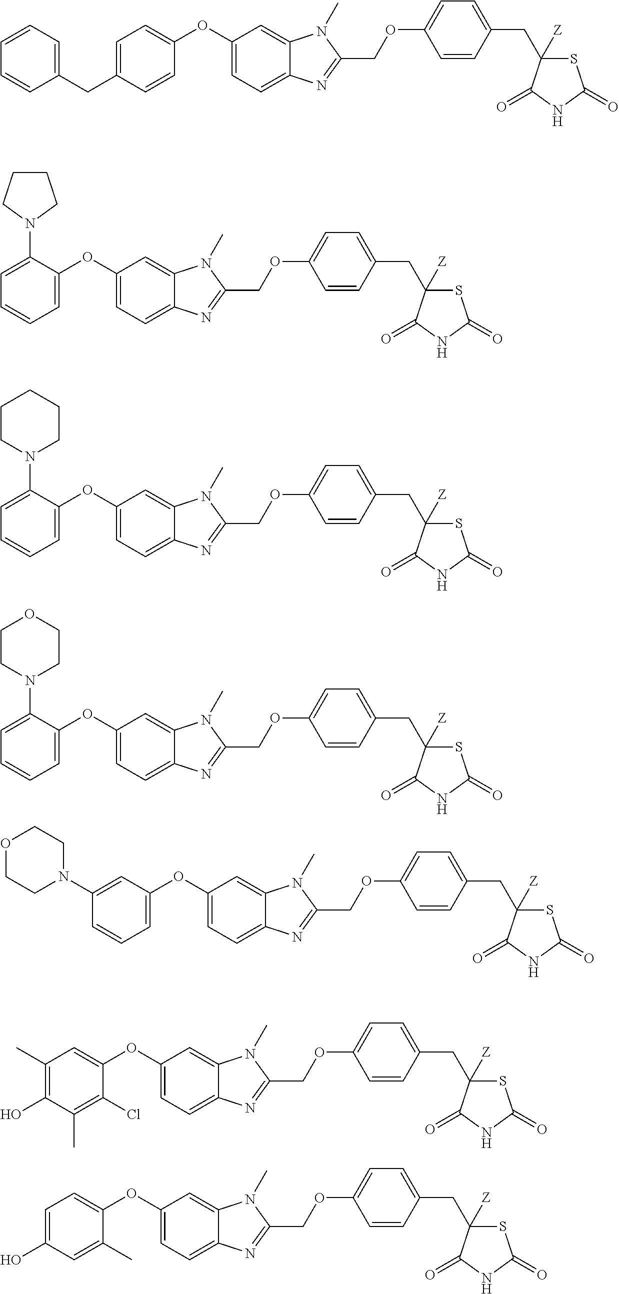

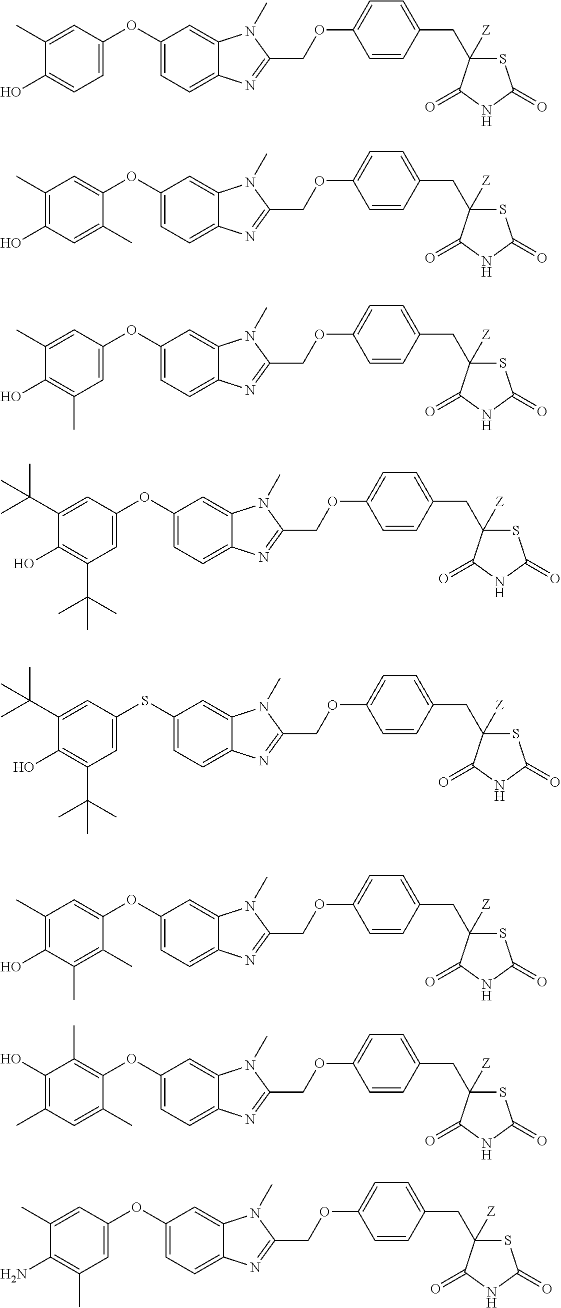

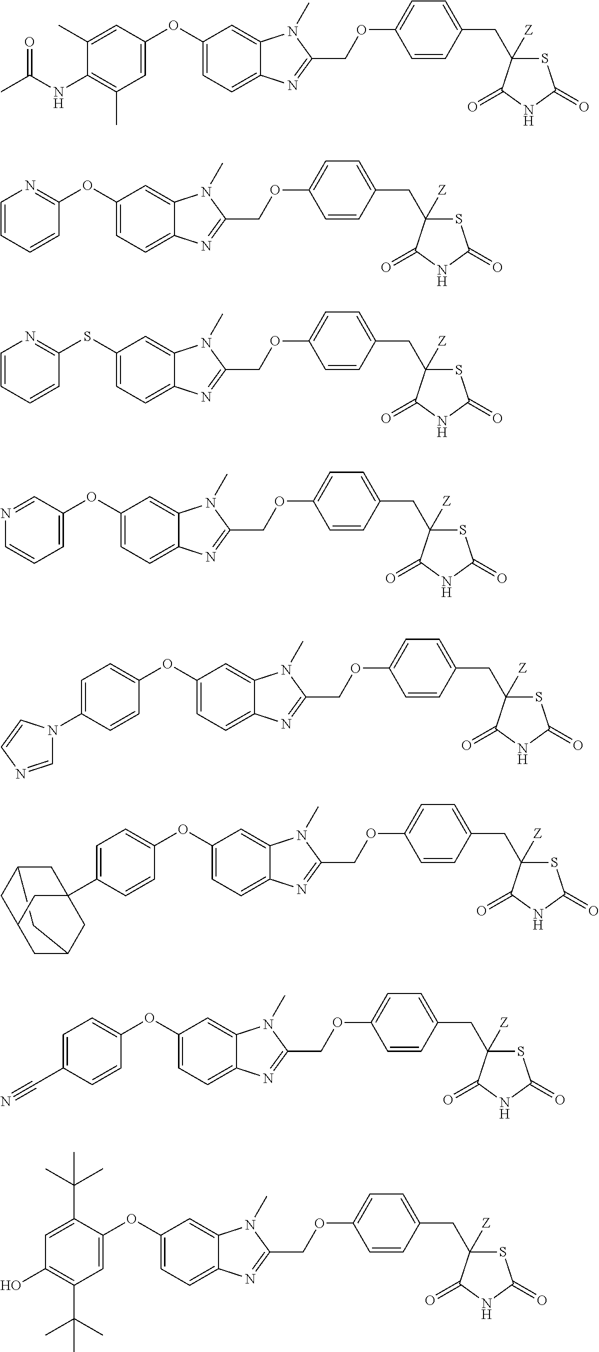

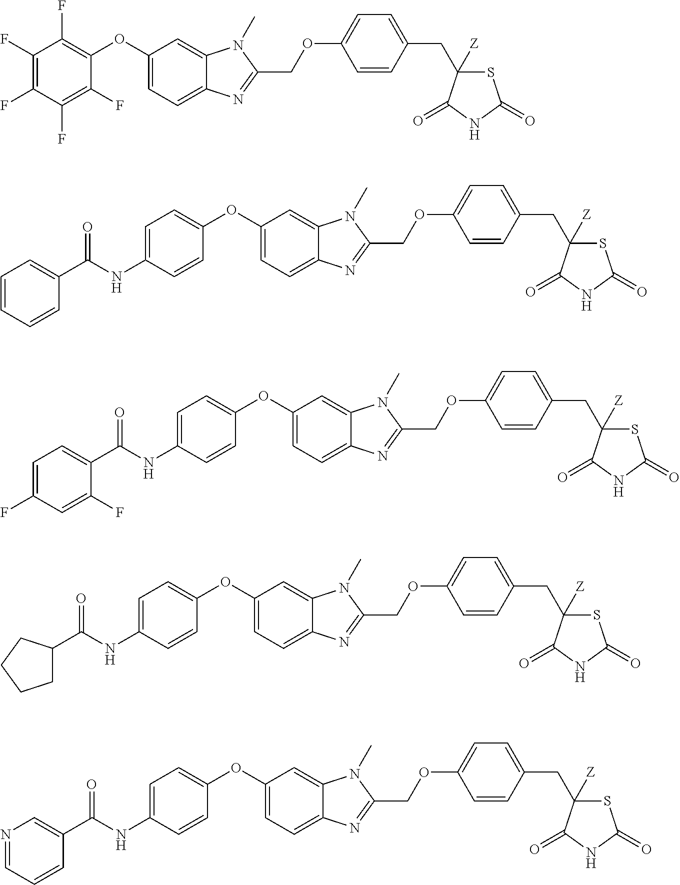

or a stereoisomer or pharmaceutically acceptable salt form thereof; where Z is as defined above for formula I.

In another aspect, the invention provides a deuterium-enriched compound having the following formula:

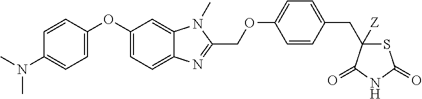

or a stereoisomer or pharmaceutically acceptable salt form thereof; where Z is as defined above for formula I. In certain embodiments, the compound is the (R)-enantiomer. In certain embodiments, the compound is the (S)-enantiomer.

In another aspect, the invention provides a deuterium-enriched compound selected from:

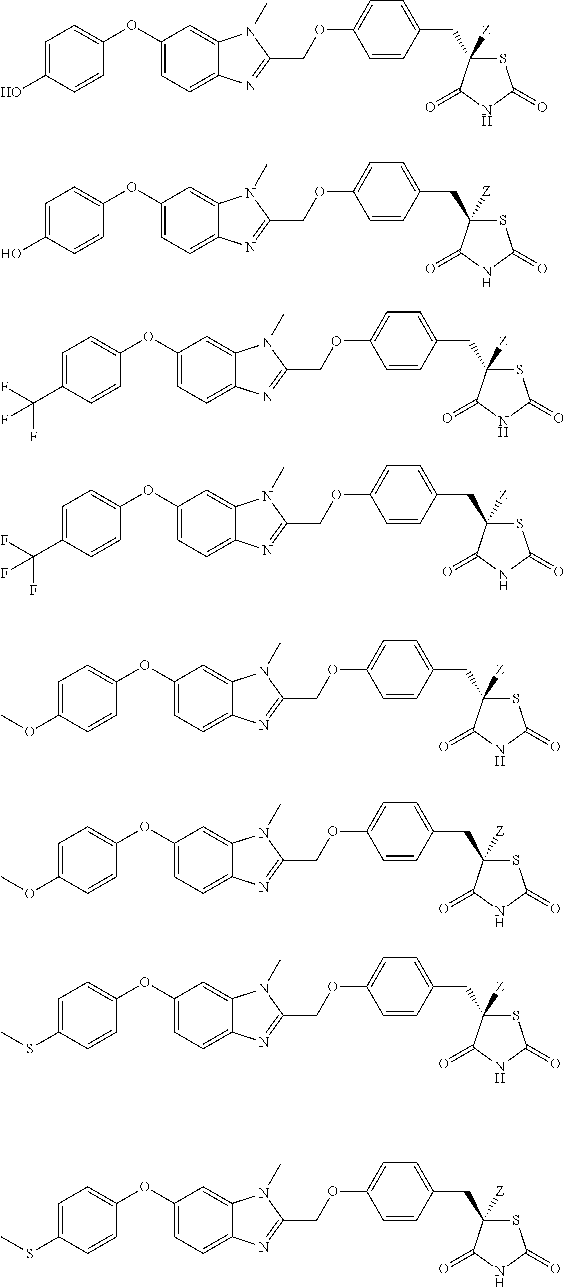

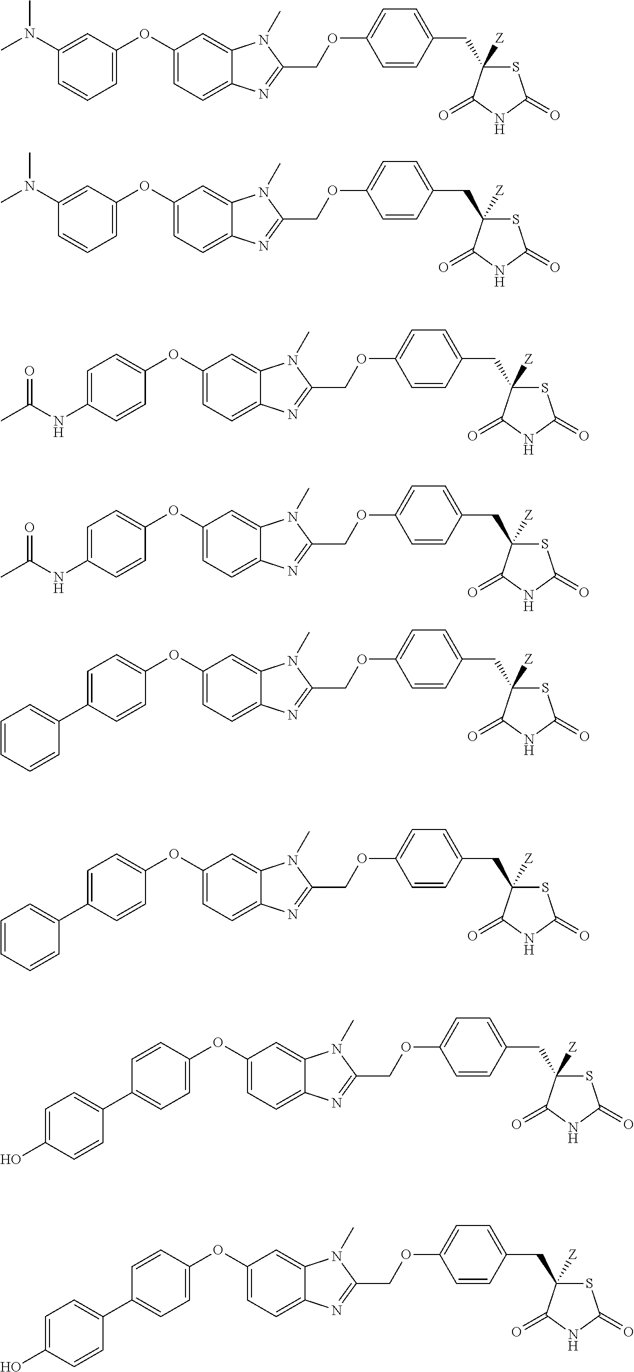

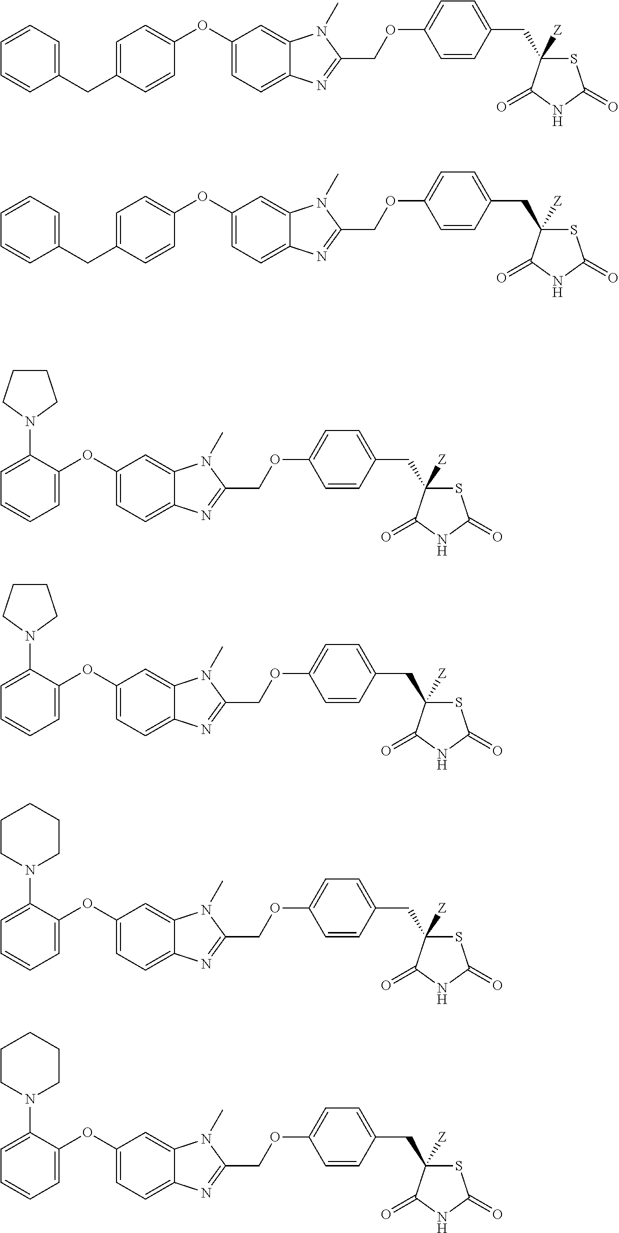

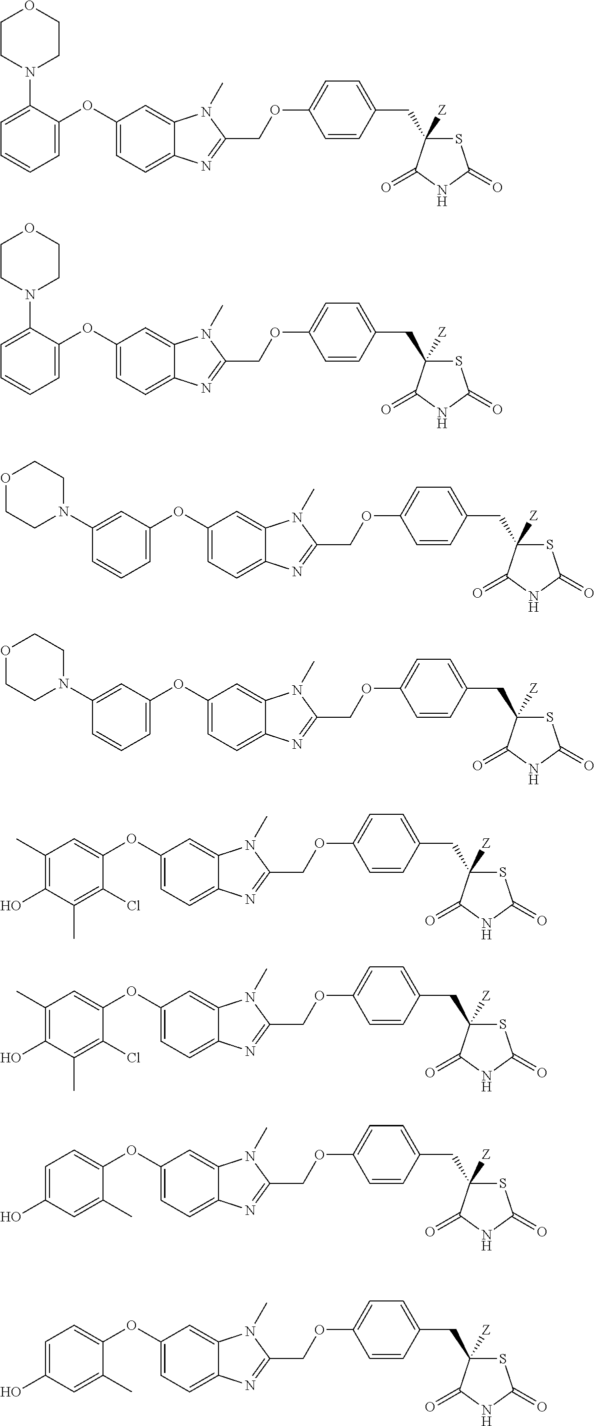

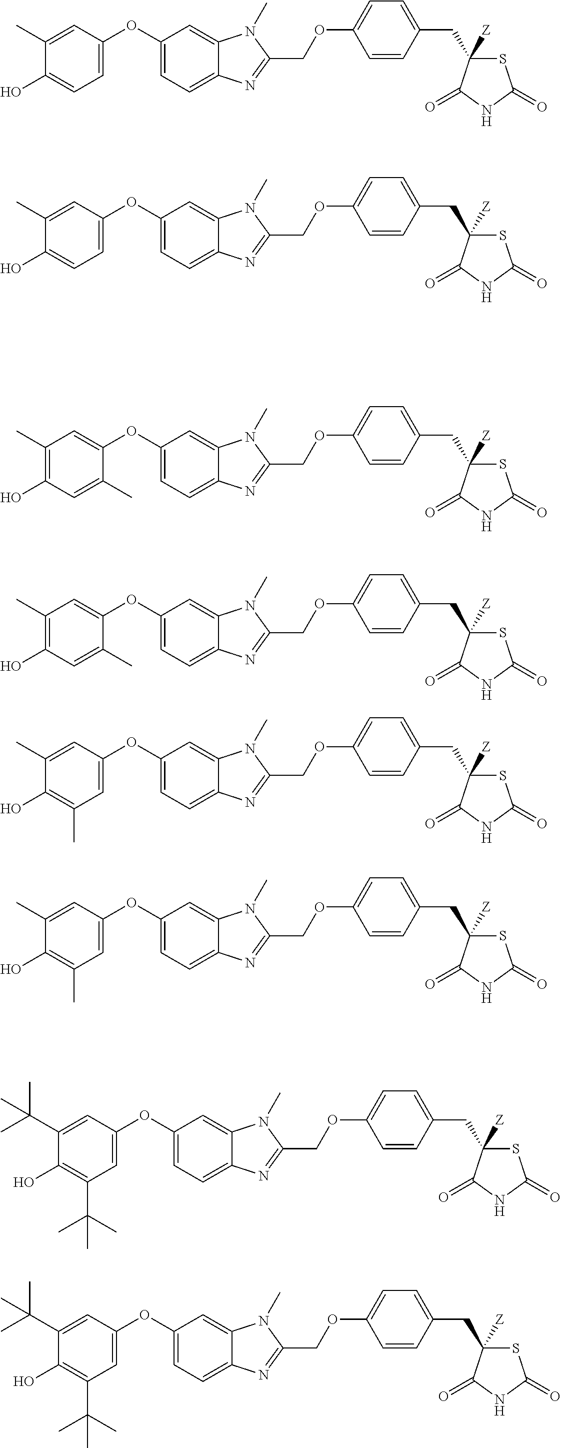

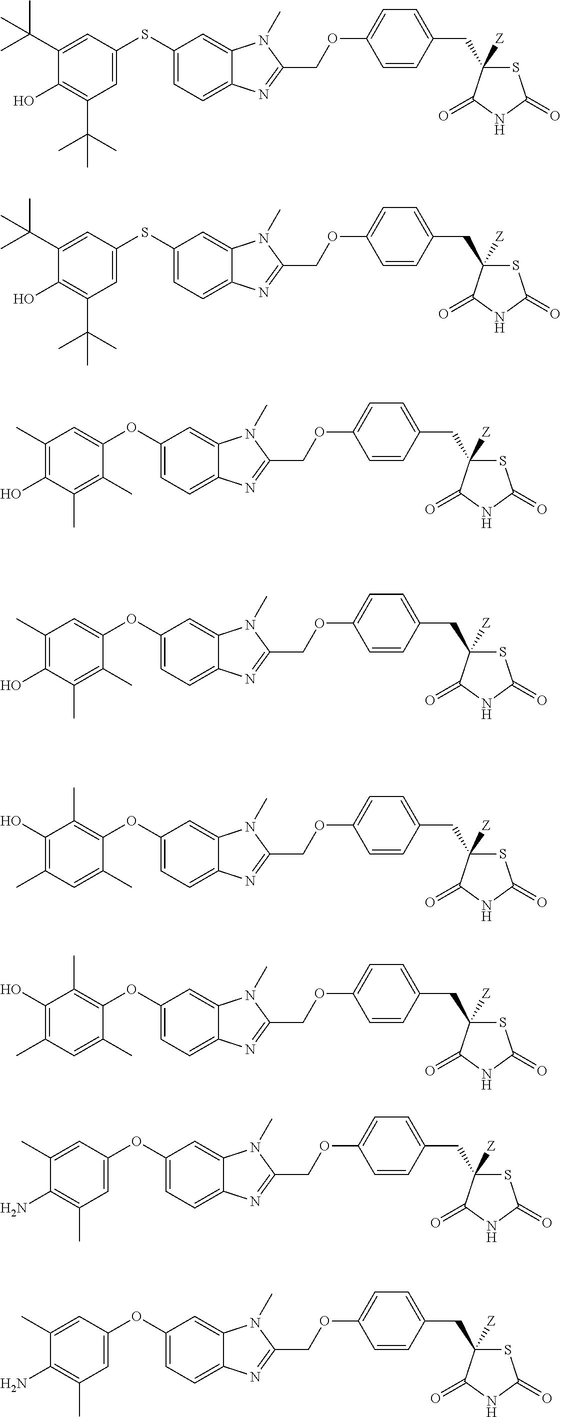

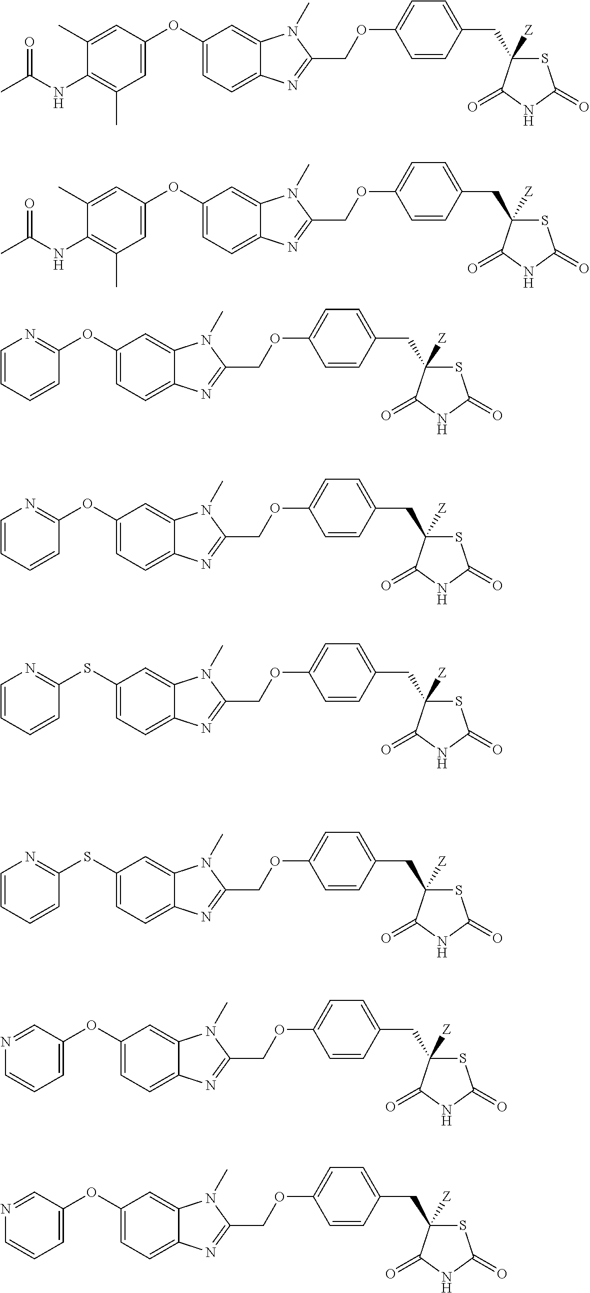

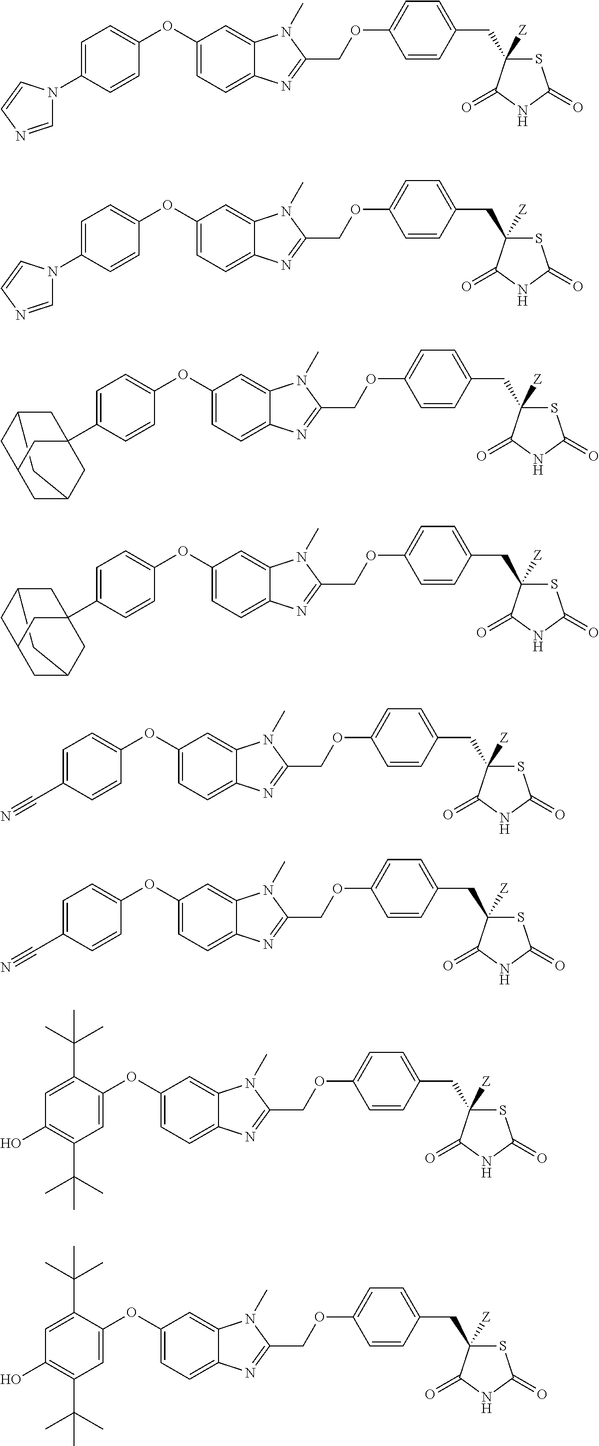

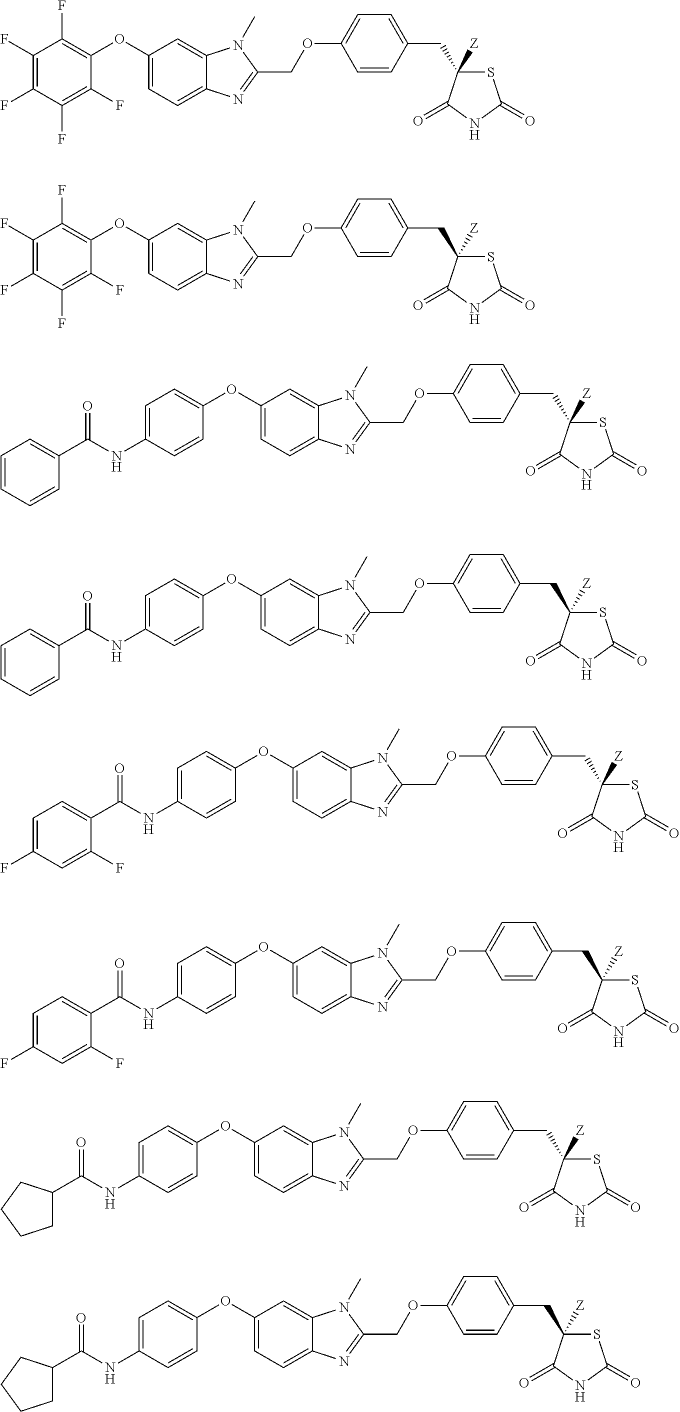

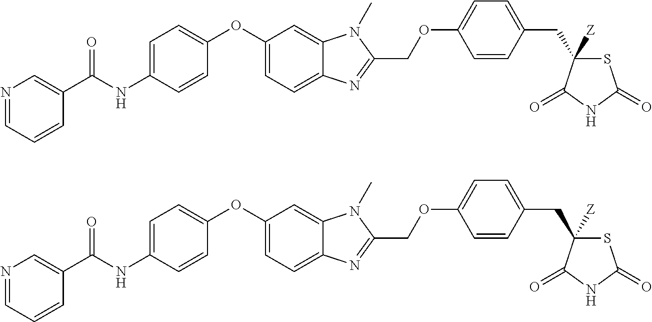

or a pharmaceutically acceptable salt form thereof; where Z is as defined above for formula I.

In another aspect, the compounds above have an enantiomeric excess, with respect to the C—Z carbon, of at least 5%.

In another aspect, the invention provides a deuterium-enriched compound selected from:

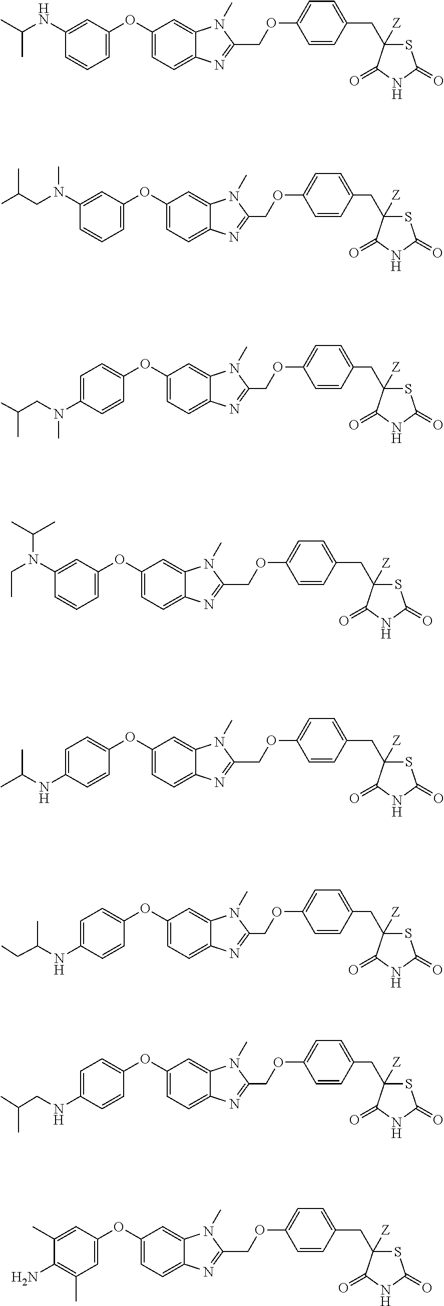

or a stereoisomer or pharmaceutically acceptable salt form thereof; wherein Z is as defined above for formula I.

In another aspect, the invention provides a deuterium-enriched compound selected from:

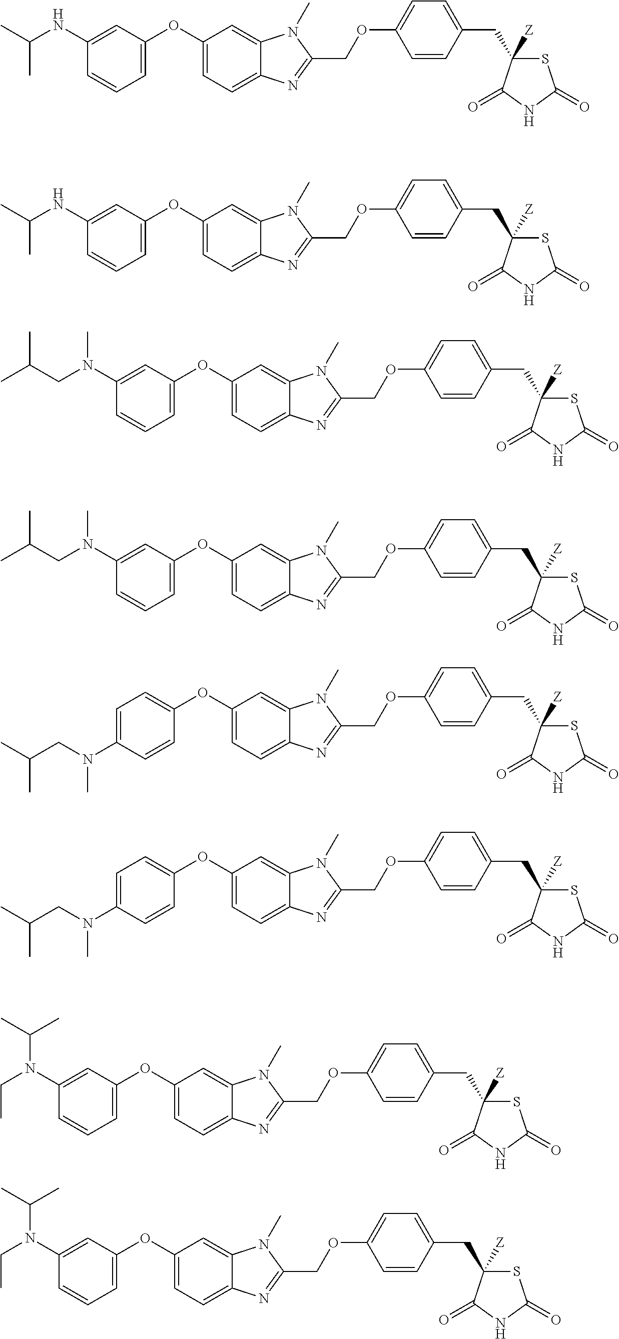

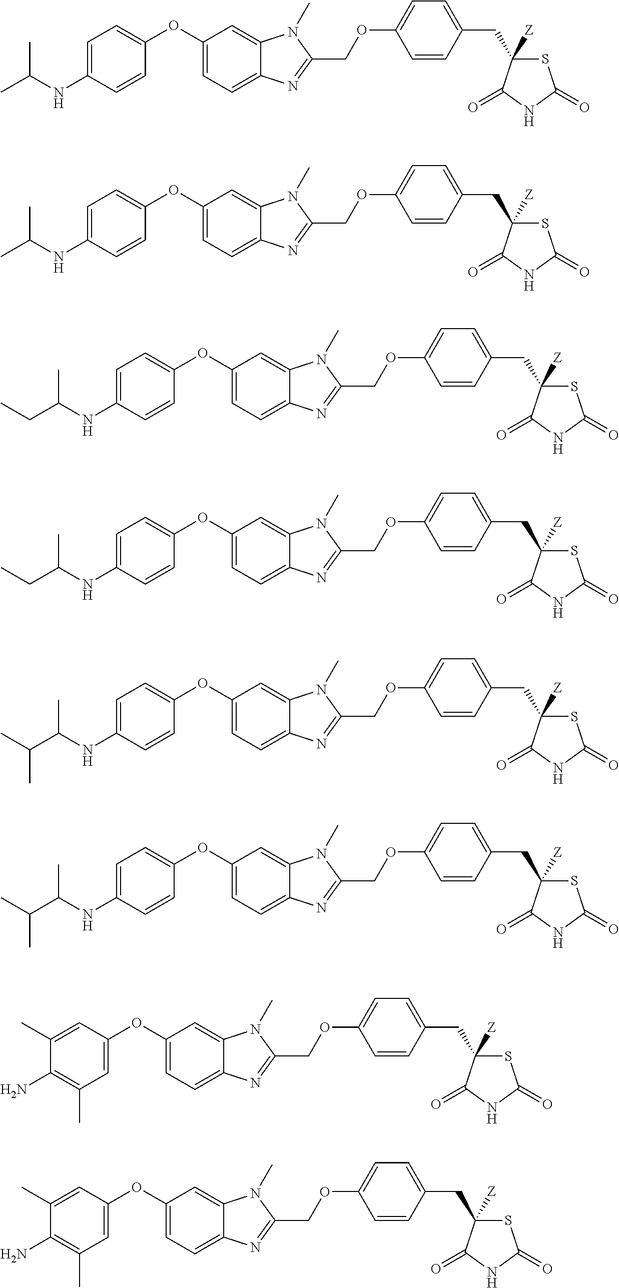

or a pharmaceutically acceptable salt form thereof; wherein Z is as defined above for formula I.

In another aspect, the compounds above have an enantiomeric excess, with respect to the C—Z carbon, of at least 5%. In yet other embodiments, the compounds above have an enantiomeric excess, with respect to the C—Z carbon, of at least 70%, 80%, 90%, 95%, 97%, 98%, or 99%.

Additional exemplary compounds are provided in the following tables, wherein variable Z is H or D, provided that the abundance of deuterium in Z is at least 30%. The R groups are as specified, and where not defined are as defined above for Formula I.

TABLE 1

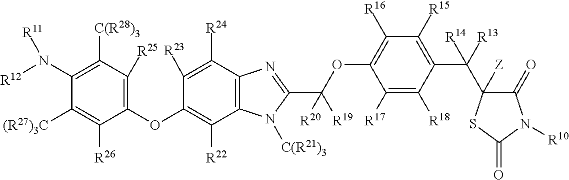

| Compound | |
|---|---|
| No. | Variable Definition |
| 1 | R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and |
| | R²⁸ = H |
| 2 | R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and |
| | R²⁸ = D |
| 3 | R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ = D |
| 4 | R¹³ = D |
| 5 | R¹³ and R¹⁴ = D |
| 6 | R¹⁵, R¹⁶, R¹⁷, and R¹⁸ = D |
| 7 | R¹³, R¹⁵, R¹⁶, R¹⁷, and R¹⁸ = D |
| 8 | R¹³, R¹⁴, R¹⁵, R¹⁶, R¹⁷, and R¹⁸ = D |
| 9 | R¹⁹ and R²⁰ = D |
| 10 | R¹³, R¹⁹, and R²⁰ = D |
| 11 | R¹³, R¹⁴, R¹⁹, and R²⁰ = D |
| 12 | R²¹, R²², R²³, and R²⁴ = D |
| 13 | R¹³, R²¹, R²², R²³, and R²⁴ = D |
| 14 | R¹³, R¹⁴, R²¹, R²², R²³, and R²⁴ = D |
| 15 | R²⁵, R²⁶, R²⁷, and R²⁸ = D |
| 16 | R¹³, R²⁵, R²⁶, R²⁷, and R²⁸ = D |
| 17 | R¹³, R¹⁴, R²⁵, R²⁶, R²⁷, and R²⁸ = D |
| 18 | R¹⁹, R²⁰, R²¹, R²², R²³, R²⁴, R²⁵, R²⁶, R²⁷, and R²⁸ = D |
| 19 | R¹³, R¹⁹, R²⁰, R²¹, R²², R²³, R²⁴, R²⁵, R²⁶, R²⁷, and R²⁸ = D |
| 20 | R¹³, R¹⁴, R¹⁹, R²⁰, R²¹, R²², R²³, R²⁴, R²⁵, R²⁶, R²⁷, and R²⁸ = D |

Table 2: The compounds corresponding to Table 1, wherein the abundance of deuterium in Z is at least 40%.

Table 3: The compounds corresponding to Table 1, wherein the abundance of deuterium in Z is at least 50%.

Table 4: The compounds corresponding to Table 1, wherein the abundance of deuterium in Z is at least 60%.

Table 5: The compounds corresponding to Table 1, wherein the abundance of deuterium in Z is at least 70%.

Table 6: The compounds corresponding to Table 1, wherein the abundance of deuterium in Z is at least 80%.

Table 7: The compounds corresponding to Table 1, wherein the abundance of deuterium in Z is at least 90%.

Table 8: The compounds corresponding to Table 1, wherein the abundance of deuterium in Z is at least 97%.

Table 9: The compounds corresponding to Table 1, wherein the abundance of deuterium in Z is about 100%.

TABLE 10

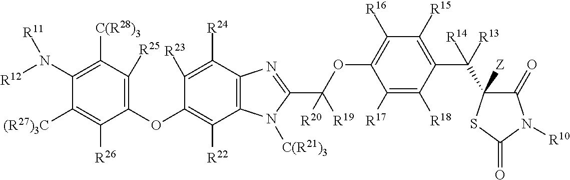

| Compound | |
|---|---|
| No. | Variable Definition |
| 1 | R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and |
| | R²⁸ = H |
| 2 | R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and |
| | R²⁸ = D |
| 3 | R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ = D |
| 4 | R¹³ = D |
| 5 | R¹³ and R¹⁴ = D |
| 6 | R¹⁵, R¹⁶, R¹⁷, and R¹⁸ = D |
| 7 | R¹³, R¹⁵, R¹⁶, R¹⁷, and R¹⁸ = D |
| 8 | R¹³, R¹⁴, R¹⁵, R¹⁶, R¹⁷, and R¹⁸ = D |
| 9 | R¹⁹ and R²⁰ = D |
| 10 | R¹³, R¹⁹, and R²⁰ = D |
| 11 | R¹³, R¹⁴, R¹⁹, and R²⁰ = D |
| 12 | R²¹, R²², R²³, and R²⁴ = D |
| 13 | R¹³, R²¹, R²², R²³, and R²⁴ = D |
| 14 | R¹³, R¹⁴, R²¹, R²², R²³, and R²⁴ = D |
| 15 | R²⁵, R²⁶, R²⁷, and R²⁸ = D |
| 16 | R¹³, R²⁵, R²⁶, R²⁷, and R²⁸ = D |
| 17 | R¹³, R¹⁴, R²⁵, R²⁶, R²⁷, and R²⁸ = D |
| 18 | R¹⁹, R²⁰, R²¹, R²², R²³, R²⁴, R²⁵, R²⁶, R²⁷, and R²⁸ = D |
| 19 | R¹³, R¹⁹, R²⁰, R²¹, R²², R²³, R²⁴, R²⁵, R²⁶, R²⁷, and R²⁸ = D |
| 20 | R¹³, R¹⁴, R¹⁹, R²⁰, R²¹, R²², R²³, R²⁴, R²⁵, R²⁶, R²⁷, and R²⁸ = D |

wherein the compounds of Table 10 have an enantiomeric excess, with respect to the C—Z carbon, of at least 5%.

Table 11: The compounds corresponding to Table 10, wherein the abundance of deuterium in Z is at least 40% and the compounds of Table 11 have an enantiomeric excess, with respect to the C—Z carbon, of at least 5%.

Table 12: The compounds corresponding to Table 10, wherein the abundance of deuterium in Z is at least 50% and the compounds of Table 12 have an enantiomeric excess, with respect to the C—Z carbon, of at least 5%.

Table 13: The compounds corresponding to Table 10, wherein the abundance of deuterium in Z is at least 60% and the compounds of Table 13 have an enantiomeric excess, with respect to the C—Z carbon, of at least 5%.

Table 14: The compounds corresponding to Table 10, wherein the abundance of deuterium in Z is at least 70% and the compounds of Table 14 have an enantiomeric excess, with respect to the C—Z carbon, of at least 5%.

Table 15: The compounds corresponding to Table 10, wherein the abundance of deuterium in Z is at least 80% and the compounds of Table 15 have an enantiomeric excess, with respect to the C—Z carbon, of at least 5%.

Table 16: The compounds corresponding to Table 10, wherein the abundance of deuterium in Z is at least 90% and the compounds of Table 16 have an enantiomeric excess, with respect to the C—Z carbon, of at least 5%.

Table 17: The compounds corresponding to Table 10, wherein the abundance of deuterium in Z is at least 97% and the compounds of Table 17 have an enantiomeric excess, with respect to the C—Z carbon, of at least 5%.

Table 18: The compounds corresponding to Table 10, wherein the abundance of deuterium in Z is about 100% and the compounds of Table 18 have an enantiomeric excess, with respect to the C—Z carbon, of at least 5%.

TABLE 19

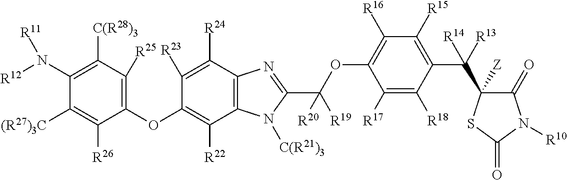

| Compound | |
|---|---|
| No. | Variable Definition |
| 1 | R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and |
| | R²⁸ = H |
| 2 | R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and |
| | R²⁸ = D |
| 3 | R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ = D |
| 4 | R¹³ = D |
| 5 | R¹³ and R¹⁴ = D |
| 6 | R¹⁵, R¹⁶, R¹⁷, and R¹⁸ = D |
| 7 | R¹³, R¹⁵, R¹⁶, R¹⁷, and R¹⁸ = D |
| 8 | R¹³, R¹⁴, R¹⁵, R¹⁶, R¹⁷, and R¹⁸ = D |
| 9 | R¹⁹ and R²⁰ = D |
| 10 | R¹³, R¹⁹, and R²⁰ = D |
| 11 | R¹³, R¹⁴, R¹⁹, and R²⁰ = D |
| 12 | R²¹, R²², R²³, and R²⁴ = D |
| 13 | R¹³, R²¹, R²², R²³, and R²⁴ = D |
| 14 | R¹³, R¹⁴, R²¹, R²², R²³, and R²⁴ = D |
| 15 | R²⁵, R²⁶, R²⁷, and R²⁸ = D |
| 16 | R¹³, R²⁵, R²⁶, R²⁷, and R²⁸ = D |
| 17 | R¹³, R¹⁴, R²⁵, R²⁶, R²⁷, and R²⁸ = D |
| 18 | R¹⁹, R²⁰, R²¹, R²², R²³, R²⁴, R²⁵, R²⁶, R²⁷, and R²⁸ = D |
| 19 | R¹³, R¹⁹, R²⁰, R²¹, R²², R²³, R²⁴, R²⁵, R²⁶, R²⁷, and R²⁸ = D |
| 20 | R¹³, R¹⁴, R¹⁹, R²⁰, R²¹, R²², R²³, R²⁴, R²⁵, R²⁶, R²⁷, and R²⁸ = D |

and the compounds of Table 19 have an enantiomeric excess, with respect to the C—Z carbon, of at least 5%.

Table 20: The compounds corresponding to Table 19, wherein the abundance of deuterium in Z is at least 40% and the compounds of Table 20 have an enantiomeric excess, with respect to the C—Z carbon, of at least 5%.

Table 21: The compounds corresponding to Table 19, wherein the abundance of deuterium in Z is at least 50% and the compounds of Table 21 have an enantiomeric excess, with respect to the C—Z carbon, of at least 5%.

Table 22: The compounds corresponding to Table 19, wherein the abundance of deuterium in Z is at least 60% and the compounds of Table 22 have an enantiomeric excess, with respect to the C—Z carbon, of at least 5%.

Table 23: The compounds corresponding to Table 19, wherein the abundance of deuterium in Z is at least 70% and the compounds of Table 23 have an enantiomeric excess, with respect to the C—Z carbon, of at least 5%.

Table 24: The compounds corresponding to Table 19, wherein the abundance of deuterium in Z is at least 80% and the compounds of Table 24 have an enantiomeric excess, with respect to the C—Z carbon, of at least 5%.

Table 25: The compounds corresponding to Table 19, wherein the abundance of deuterium in Z is at least 90% and the compounds of Table 25 have an enantiomeric excess, with respect to the C—Z carbon, of at least 5%.

Table 26: The compounds corresponding to Table 19, wherein the abundance of deuterium in Z is at least 97% and the compounds of Table 26 have an enantiomeric excess, with respect to the C—Z carbon, of at least 5%.

Table 27: The compounds corresponding to Table 19, wherein the abundance of deuterium in Z is about 100% and the compounds of Table 27 have an enantiomeric excess, with respect to the C—Z carbon, of at least 5%.

A hydrogen atom present in any compound of the invention is optionally replaced by D (i.e., the position is enriched as described for variable Z).

The invention is based on stabilizing 2,4-thiazolidinediones and 2,4-oxazolidinediones via deuteration at the 5-position. The C-D bond at the 5-position is stronger than the naturally occurring C—H bond. The 5-deuterium is expected to slow the racemization of the stereogenic center at the 5-position.

Hydrogen atoms are present in formulae described herein, such as I-XIIb₁. As such, the present deuterium-enriched compounds can be further enriched beyond the 5-position. For example, in formula I the phenyl ring and/or groups R¹, R², R⁴⁻⁶, A, E, and R^{a-d} can either be substituted with D (e.g., the phenyl ring or R¹) or fully replaced by D (e.g., R⁶). Additional enrichment of the compounds of the invention can include enrichment at one additional position or multiple positions. Examples of this enrichment include (a) at least 10%, (b) at least 20%, (c) at least 30%, (d) at least 40%, (e) at least 50%, (f) at least 60%, (g) at least 70%, (h) at least 80%, (i) at least 90%, (j) at least 95%, (k) at least 97%, and (l) about 100%. The percentage enriched can correspond to one single position (e.g., 10% of R²=D) or a group of positions (e.g., 10% of the R² and R⁶ positions=D).

For other compounds of the invention, enrichment beyond the 5-position includes the presence of at least one additional deuterium. For example, enrichment can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc., up to the total number of hydrogen atoms present and depending on the number of hydrogens present.

In another aspect, the invention provides isolated or purified compounds. The isolated or purified compound is a group of molecules (e.g., an isolated compound) whose deuterium levels are above the naturally occurring levels. The isolated or purified compounds of the invention can be obtained by techniques known to those of skill in the art.

Isolated means that the non-naturally occurring compound is purified (e.g., from the reaction solution in which it was prepared). Examples of the purity of the isolated compound (could be more than one type of compound) include, but are not limited to, at least 50%, 55%, 60%, 65%, 70%, 75%, 80%, 85%, 90%, 95%, to 100% with respect to non-deuterium-enriched components being present.

In another aspect, the invention provides mixture of compounds, which means that more than one type of deuterated compound is being claimed.

In another aspect, the invention provides compositions comprising compounds of the invention. The compositions require the presence of a compound of the invention that is greater than its natural abundance. For example, the compositions of the invention can comprise (a) a μg of a compound of the invention; (b) from 1-10 μg; (c) a mg; (d) from 1-10 mg; (e) a gram; (f) from 1-10 grams; (g) from 1-100 grams; and, (h) a kg.

In another aspect, the invention provides an amount of a novel compound of the invention. Examples of amounts include, but are not limited to (a) at least 0.01, 0.02, 0.03, 0.04, 0.05, 0.1, 0.2, 0.3, 0.4, 0.5, to 1 mole, (b) at least 0.1 moles, and (c) at least 1 mole of the compound. The present amounts also cover lab-scale (e.g., gram scale including 1, 2, 3, 4, 5 g, etc.), kilo-lab scale (e.g., kilogram scale including 1, 2, 3, 4, 5 kg, etc.), and industrial or commercial scale (e.g., multi-kilogram or above scale including 100, 200, 300, 400, 500 kg, etc.) quantities as these will be more useful in the actual manufacture of a pharmaceutical. Industrial/commercial scale refers to the amount of product that would be produced in a batch that was designed for clinical testing, formulation, sale/distribution to the public, etc.

In another aspect, the invention provides novel pharmaceutical compositions, comprising: a pharmaceutically acceptable carrier and a therapeutically effective amount of a deuterium-enriched compound of the invention.

In another aspect, the invention provides novel pharmaceutical compositions, comprising: a pharmaceutically acceptable carrier and a deuterium-enriched compound of the invention.

### II. EXEMPLARY GENERAL PROCEDURES FOR SYNTHESIS OF DEUTERIUM-ENRICHED COMPOUNDS

The hydrogens present on the 5-deuterium enriched 2,4-thiazolidinediones and 2,4-oxazolidinediones of the invention have different capacities for exchange with deuterium. For example, the hydrogen atom for Z is exchangeable under basic conditions (e.g., NaOD) in the presence of D₂O. Hydroxy and amino hydrogen atoms, for example R¹⁰⁻¹² shown in Table 1, are exchangeable in H₂O/D₂O. The remaining non-hydroxy and non-amino hydrogen atoms are not easily exchangeable for deuterium atoms, though some may be depending on the specific moieties selected (e.g., hydrogrens adjacent to carbonyl groups are expected to be base exchangeable). Deuterium atoms may be incorporated into non-exchangeable positions by the use of deuterated starting materials or intermediates via the known synthetic methods for the synthesis of 5-deuterium enriched 2,4-thiazolidinediones and 2,4-oxazolidinediones (e.g., 5-(4-((6-(4-amino-3,5-dimethylphenoxy)-1-methyl-1H-benzo[d]imidazol-2-yl)methoxy)benzyl)-5-deutero-thiazolidine-2,4-dione), as described in U.S. Pat. Nos. 6,432,993 and 8,263,631 and International Patent Application Publication No. WO 2008/099944, the contents of which are incorporated in their entirety herein by reference. It is contemplated that the presently described deuterated 5-deuterium enriched 2,4-thiazolidinediones and 2,4-oxazolidinediones can be prepared by incorporating deuterated starting materials into the synthetic route of U.S. Pat. No. 6,432,993. Alternatively, deuterium is expected to be incorporated at the exchangeable and acidic positions of the final compound (e.g., 5-position).

Scheme 1 below provides an exemplary synthetic route for preparing deuterium-enriched compounds of the invention.

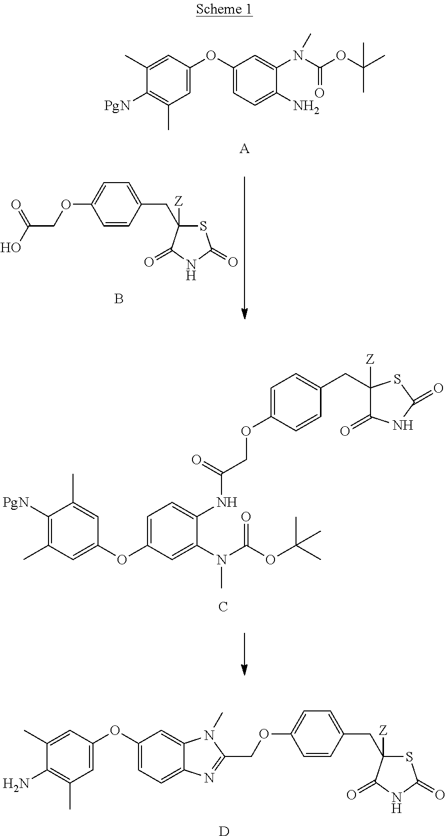

The compounds of the invention can be obtained by coupling protected phenoxy-phenyl-carbamate A (wherein Pg is an acid-labile protecting group) with acetic acid compound B, typically in the presence of a coupling agent (e.g., diethyl cyanophosphate), and a base (e.g., triethylamine) to arrive at amide C. Amide C can then be both deprotected and cyclized to compound D in the presence of a strong acid (e.g., HCl). In compound B, Z can be H, D, or partially D (e.g., see deuterium-enrichment percentages provided previously). Compound B, when D is present, can be the racemic mixture of R and S stereoisomers, or can be a specific isomer (or a mixture of isomers wherein the R or S is enriched above 50%). When Z═H in B, deuterium can be introduced into C or D by contacting either C or D with a base (e.g., NaOD) in the presence of D₂O. Chiral chromatography or other known chiral isolation techniques can then be used to resolve the stereoisomers.

Scheme 2 provides an alternative route to prepare deuterium-enriched compounds of the invention.

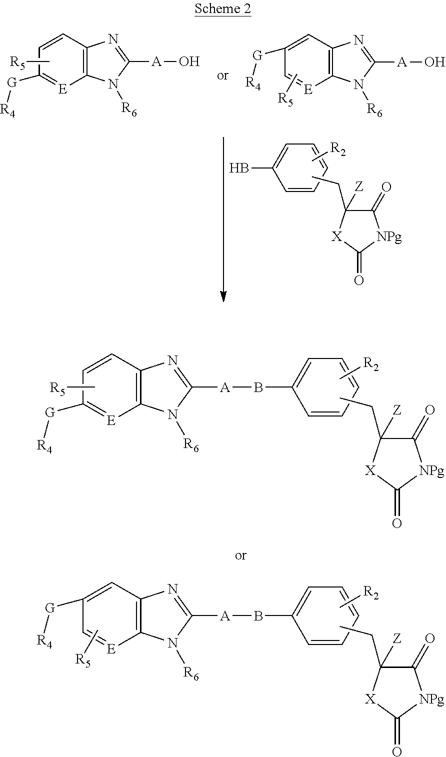

In Scheme 2, R¹-A-OH is coupled with HB-phenyl-2,4-thiazolidinedione/2,4-oxazolidinedione in an inert solvent (e.g., hexanes) in the presence of a phosphine (e.g., tributylphosphine or triphenylphosphine) and an azodicarboxylic acid compound (e.g., diethyl azodicarboxylate or 1,1′-(azodicarbonyl)dipiperidine). Typically any amine or hydroxyl groups are protected. After coupling, the protecting groups can be removed using known technology (e.g., acid removal and hydrogenation (e.g., H₂/Pd/C).

Scheme 3 describes another alternative route to prepare deuterium-enriched compounds of the invention.

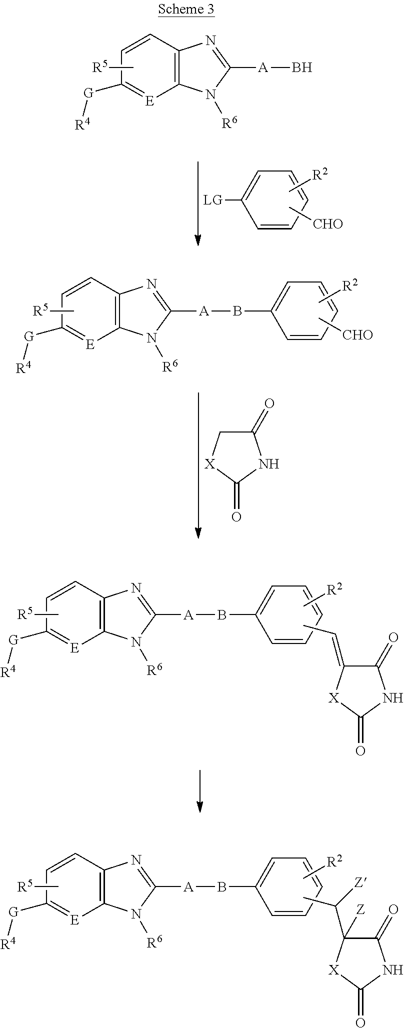

In Scheme 3, the A-BH compound is coupled with a benzaldehyde, typically in the presence of a base (e.g., NaOH) and inert solvent (e.g., hexanes). The aldehyde of the coupled product is then reacted with a 2,4-thiazolidinedione/2,4-oxazolidinedione. The resulting alkenyl compound can then be reduced (e.g., H₂ or D₂ in presence of Pd/C) to the desired product. Any protecting groups that are not reducible can be removed using known techniques (e.g., acid removal). If D₂ and Pd/C are used, then a D can be introduced at the Z and Z′ positions. If H₂ is used, then a D can be introduced for Z by contacting with a base (e.g., NaOD) in D₂O.

In the above schemes, if a racemic starting material (e.g., Z=D) is used or if stereospecificity is lost during a reaction, the resulting deuterated racemic mixture should be separable using known isolation techniques (e.g., chiral chromatography or crystallization).

### III. THERAPEUTIC APPLICATIONS

The invention provides methods of using deuterium-enriched compounds described herein to treat medical disorders. The deuterium-enriched compound can be, for example, a compound of formula I, XII, XV, XVIII, or one of the other deuterium-enriched compounds described in Section I above. Various aspects of the invention pertaining to treating medical disorders are described below.

One aspect of the invention provides methods of treating, preventing, and/or managing various diseases or disorders using a deuterium-enriched compound provided herein, or a pharmaceutically acceptable salt or stereoisomer thereof. Without being limited by a particular theory, compounds provided herein are expected to have insulin resistance improving action, anti-inflammatory action, immunomodulatory action, aldose reductase inhibitory action, 5-lipoxygenase inhibitory action, lipid peroxide production inhibitory action, peroxisome proliferator activated receptor (which will hereinafter be abbreviated as “PPAR”) activating action, anti-osteoporosis action, leukotrienes antagonism, fat-cell formation promoting action, cancer-cell proliferation inhibitory action, and/or calcium antagonism.

Another aspect of the invention provides an amount of a deuterium-enriched compound of the invention as described above for use in therapy. Yet another aspect of the invention provides for the use of an amount of a deuterium-enriched compound of the invention for the manufacture of a medicament.

Exemplary disorders for treatment include, for example, cancer, a neurological disorder, a respiratory disorder, a metabolic disorder, an inflammatory disorder, cardiovascular disorder, and dermatological disorder. In certain embodiments, the disorder is a cancer, neurological disorder, respiratory disorder, or metabolic disorder. The treatment method may comprise, for example, administering to a patient in need thereof a therapeutically effective amount of a deuterium-enriched compound described herein, such as a compound of formula I, XII, XV, or XVIII, to treat the disorder.

### Treating Neurological Disorders

Accordingly, one aspect of the invention provides a method of treating a neurological disorder selected from the group consisting of Alzheimer's disease, Parkinson's disease, amyotrophic lateral sclerosis, Friedreich's ataxia, autism spectrum disorder, depression, mild cognitive impairment, neurodegeneration, adrenoleukodystrophy, Huntington's disease, stroke, traumatic brain injury, substance abuse, spinal cord injury, neuronal injury, and major depression or bipolar disorder comorbid with metabolic syndrome. The method comprises administering to a patient in need thereof a therapeutically effective amount of a deuterium-enriched compound described herein, such as a compound of Formula I, XII, XIIa, XIIb, XV, or XVIII, to treat the disorder. In certain embodiments, the neurological disorder is selected from the group consisting of Alzheimer's disease, Parkinson's disease, amyotrophic lateral sclerosis, Friedreich's ataxia, depression, mild cognitive impairment, neurodegeneration, adrenoleukodystrophy, and Huntington's disease. In certain other embodiments, the neurological disorder is Alzheimer's disease.

In certain other embodiments, the neurological disorder is a cognitive disorder, such as cognitive impairment and/or memory impairment. The cognitive impairment may be, for example, cognitive impairment associated with Alzheimer's disease.

In certain embodiments, the substance abuse is one or more of alcohol craving, heroin dependence, and nicotine dependence.

### Treating Cancer

Another aspect of the invention provides a method of treating cancer. The method comprises administering to a patient in need thereof a therapeutically effective amount of a deuterium-enriched compound described herein, such as a compound of Formula I, XII, XIIa, XIIb, XV, or XVIII, to treat the cancer.

In certain embodiments, the cancer is lung cancer, hepatocellular carcinoma, astrocytoma, glioma, glioblastoma, meningioma, liver cancer, lymphoma, melanoma, multiple myeloma, pancreatic cancer, colorectal cancer, pituitary cancer, thyroid cancer, esophageal cancer, or prostate cancer. In certain embodiments, the cancer is non-small cell lung cancer or hepatocellular carcinoma. In certain other embodiments, the cancer is colorectal cancer. In certain other embodiments, the cancer is esophageal cancer. In certain other embodiments, the cancer is non-small cell lung cancer, such as metastatic non-small cell lung cancer. In certain other embodiments, the cancer is thyroid cancer, such as anaplastic thyroid cancer. In certain other embodiments, the cancer is a solid tumor.

In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratosis, acute lymphocytic leukemia, acute myeloid leukemia, adenoid cystic carcinoma, adenoma, adenosarcoma, adenosquamous carcinoma, anal canal cancer, anal cancer, anorectal cancer, astrocytic tumor, Bartholin's gland carcinoma, basal cell carcinoma, biliary cancer, bone cancer, bone marrow cancer, bronchial cancer, bronchial gland carcinoma, carcinoid, cholangiocarcinoma, chorioid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastoma, hemangioendothelioma, hemangioma, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanoma, lymphoma, male genital cancer, malignant melanoma, malignant mesothelial tumor, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma, nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumor, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T-cell leukemia, tongue cancer, undifferentiated carcinoma, ureter cancer, urethral cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is a neoplasm. In certain other embodiments, the cancer is a lymphoma.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

### Treating Respiratory Disorders

Another aspect of the invention provides a method of treating a respiratory disorder. The method comprises administering to a patient in need thereof a therapeutically effective amount of a deuterium-enriched compound described herein, such as a compound of Formula I, XII, XIIa, XIIb, XV, or XVIII, to treat the disorder. In certain embodiments, the respiratory disorder is chronic obstructive pulmonary disease, asthma, bronchitis, cystic fibrosis, pulmonary edema, pulmonary embolism, pulmonary arterial hypertension, pneumonia, pulmonary sarcoidosis, silicosis, pulmonary fibrosis, respiratory failure, acute respiratory distress syndrome, emphysema, chronic bronchitis, tuberculosis, lung cancer, or a chronic respiratory condition. In certain embodiments, the respiratory disorder is chronic obstructive pulmonary disease, asthma, or a chronic respiratory condition. In certain other embodiments, the respiratory disorder is chronic obstructive pulmonary disease. In yet other embodiments, the respiratory disorder is bronchitis, cystic fibrosis, pulmonary edema, pulmonary embolism, pneumonia, pulmonary sarcoidosis, silicosis, pulmonary fibrosis, respiratory failure, acute respiratory distress syndrome, emphysema, chronic bronchitis, tuberculosis, or lung cancer. In certain embodiments, the asthma is mild asthma, moderate asthma, severe asthma, or steroid-resistant asthma.

In certain embodiments, the deuterium-enriched compound is administered by routes other than pulmonary administration. In certain embodiments, the deuterium-enriched compound is administered by oral administration, sublingual administration, sublabial administration, rectal administration, injection, or transdermal administration. In certain other embodiments, the deuterium-enriched compound is administered by pulmonary administration.

### Treating Metabolic Disorders

Another aspect of the invention provides a method of treating a metabolic disorder selected from the group consisting of diabetes, insulin resistance, gout, hyperglycemia, nonalcoholic steatohepatitis, nonalcoholic fatty liver disease, diabetic retinopathy, diabetic neuropathy, diabetic nephropathy, beta-cell depletion, insulin resistance in a patient with congenital adrenal hyperplasia treated with a glucocorticoid, dysmetabolism in peritoneal dialysis patients, reduced insulin secretion, improper distribution of brown fat cells and white fat cells, obesity, or improper modulation of leptin levels. The method comprises administering to a patient in need thereof a therapeutically effective amount of a deuterium-enriched compound described herein, such as a compound of Formula I, XII, XIIa, XIIb, XV, or XVIII, to treat the disorder. In certain embodiments, the metabolic disorder is diabetes, insulin resistance, gout, hyperglycemia, nonalcoholic steatohepatitis, nonalcoholic fatty liver disease, diabetic retinopathy, diabetic neuropathy, diabetic nephropathy, beta-cell depletion, reduced insulin secretion, improper distribution of brown fat cells and white fat cells, obesity, or improper modulation of leptin levels. In certain other embodiments, the metabolic disorder is inadequate glucose tolerance. In certain other embodiments, the metabolic disorder is nonalcoholic fatty liver disease. In certain other embodiments, the metabolic disorder is nonalcoholic steatohepatitis. In certain other embodiments, the metabolic disorder is diabetes, such as insulin-dependent diabetes or non-insulin dependent diabetes. In certain embodiments, the metabolic disorder is Type II diabetes. In yet other embodiments, the diabetes is diabetes mellitus or gestational diabetes. In certain other embodiments, the metabolic disorder is beta-cell loss treatable by beta-cell regeneration. In certain embodiments, the metabolic disorder is a complication of diabetes.

In yet other embodiments, the metabolic disorder is further selected from metabolic syndrome, pathological glucose tolerance, retinopathy, or a diabetic ulcer.

### Treating a Symptom of Hepatitis

Another aspect of the invention provides a method of treating a symptom of hepatitis. The method comprises administering to a patient in need thereof a therapeutically effective amount of a deuterium-enriched compound described herein, such as a compound of Formula I, XII, XIIa, XIIb, XV, or XVIII, to treat a symptom of hepatitis.

### Treating Cardiovascular Disease

Another aspect of the invention provides a method of treating a cardiovascular disease. The method comprises administering to a patient in need thereof a therapeutically effective amount of a deuterium-enriched compound described herein, such as a compound of Formula I, XII, XIIa, XIIb, XV, or XVIII, to treat the cardiovascular disease. In certain embodiments, the cardiovascular disease is hypertension, hyperlipidemia, atherosclerosis, improper vascular function, dyslipidemia, stenosis, restenosis, myocardial infarction, stroke, intracranial hemorrhage, acute coronary syndrome, stable angina pectoris, or unstable angina pectoris. In certain other embodiments, the cardiovascular disorder is intracranial hemorrhage, acute coronary syndrome, stable angina pectoris, or unstable angina pectoris.

In another aspect, the invention provides a method for preventing stroke in a patient. The method comprises administering to a patient in need thereof a therapeutically effective amount of a deuterium-enriched compound described herein, such as a compound of Formula I, XII, XIIa, XIIb, XV, or XVIII, to prevent said stroke.

The method of treatment or the method of prevention may involve a patient at risk for central nervous system ischemic stroke, or may involve a patient at risk for stroke due to cardiovascular disease.

### Reducing the Amount of a Triglyceride or Low-Density Lipoprotein

Another aspect of the invention provides a method of reducing the amount of a triglyceride or low-density lipoprotein (LDL) in a patient. The method comprises administering to a patient in need thereof an effective amount of a deuterium-enriched compound described herein, such as a compound of Formula I, XII, XIIa, XIIb, XV, or XVIII, to reduce the amount of a triglyceride or LDL in the patient.

In certain embodiments, the method provides a reduction of at least 1%, 5%, 10%, or 25% in the amount of a triglyceride or low-density lipoprotein (LDL) in the patient.

### Increasing the Amount of High-Density Lipoprotein

Another aspect of the invention provides a method of increasing the amount of high-density lipoprotein (HDL) in a patient. The method comprises administering to a patient in need thereof a therapeutically effective amount of a deuterium-enriched compound described herein, such as a compound of Formula I, XII, XIIa, XIIb, XV, or XVIII, to increase the amount of HDL in the patient.

In certain embodiments, the method provides an increase of at least 1%, 5%, 10%, or 25% in the amount of high-density lipoprotein (HDL) in a patient.

### Treating an Inflammatory or Immune-Mediated Disorder

Another aspect of the invention provides a method of treating an inflammatory or immune-mediated disorder selected from the group consisting of chronic kidney disease, arthritis, a primary cicatricial alopecia, lung fibrosis, multiple sclerosis, endotoxemia, sepsis, septic shock, laminitis, inflammatory bowel disease, colitis, Crohn's disease, rheumatoid arthritis, lupus, myasthenia gravis, vasculitis, chronic pancreatitis, a hyperproliferative skin disorder, an inflammatory skin disorder, and a dermatological condition. The method comprises administering to a patient in need thereof a therapeutically effective amount of a deuterium-enriched compound described herein, such as a compound of Formula I, XII, XIIa, XIIb, XV, or XVIII, to treat the disorder. In certain embodiments, the inflammatory or immune-mediated disorder is selected from the group consisting of chronic kidney disease, arthritis, a primary cicatricial alopecia, lung fibrosis, multiple sclerosis, endotoxemia, sepsis, septic shock, laminitis, inflammatory bowel disease, colitis, Crohn's disease, rheumatoid arthritis, lupus, myasthenia gravis, vasculitis, chronic pancreatitis, a hyperproliferative skin disorder, an inflammatory skin disorder, and a dermatological condition. In certain embodiments, the chronic kidney disease may be, for example, polycystic kidney disease (such as autosomal dominant or autosomal recessive).

### Treating a Dermatological Disorder

Another aspect of the invention provides a method of treating a dermatological disorder selected from the group consisting of psoriasis, atopic dermatitis, acne, leukoplakia, scleroderma, and a skin malignancy. The method comprises administering to a patient in need thereof a therapeutically effective amount of a deuterium-enriched compound described herein, such as a compound of Formula I, XII, XIIa, XIIb, XV, or XVIII, to treat the disorder.

### Modulating Expression of Pro-Inflammatory Cytokines

Another aspect of the invention provides a method of modulating expression of a pro-inflammatory cytokine (e.g., TNFα, IL-1β, or IL-6) in a patient suffering from an inflammatory disorder. The method comprises administering to a patient in need thereof an effective amount of a deuterium-enriched compound described herein, such as a compound of Formula I, XII, XIIa, XIIb, XV, or XVIII, to modulate expression of the pro-inflammatory cytokine. In certain embodiments, the pro-inflammatory cytokine is TNFα.

Another aspect of the invention provides a method of modulating expression of an anti-inflammatory cytokine in a patient suffering from an inflammatory disorder. The method comprises administering to a patient in need thereof an effective amount of a deuterium-enriched compound described herein, such as a compound of Formula I, XII, XIIa, XIIb, XV, or XVIII, to modulate expression of the anti-inflammatory cytokine

### Modulating Macrophage Function

Another aspect of the invention provides a method of modulating macrophage function in a patient suffering from an infection. The method comprises administering to a patient in need thereof an effective amount of a deuterium-enriched compound described herein, such as a compound of Formula I, XII, XIIa, XIIb, XV, or XVIII, to modulate macrophage function.

### Method of Promoting Wound Healing

Another aspect of the invention provides a method of promoting wound healing. The method comprises administering to a patient in need thereof a therapeutically effective amount of a deuterium-enriched compound described herein, such as a compound of Formula I, XII, XIIa, XIIb, XV, or XVIII, to promote wound healing.

### Treating Skin Defects

Another aspect of the invention provides a method of treating skin defects caused by exposure to ultraviolet radiation. The method comprises administering to a patient in need thereof a therapeutically effective amount of a deuterium-enriched compound described herein, such as a compound of Formula I, XII, XIIa, XIIb, XV, or XVIII, to treat skin defects caused by exposure to ultraviolet radiation.

### Method of Modulating Stem Cell Differentiation

Another aspect of the invention provides a method of modulating stem cell differentiation, such as in a patient. The method comprises exposing a stem cell to a deuterium-enriched compound described herein, such as a compound of Formula I, XII, XIIa, XIIb, XV, or XVIII, to modulate stem cell differentiation. In certain embodiments, the method modulates stem cell differentiation in a patient by administering to the patient an effective amount of a compound herein.

### Regenerative Medicine

Also provided are methods of using compounds herein for therapy comprising regenerative medicine.

### Preventing Medical Disorders

Also provided are methods of preventing a medical disorder in a patient. The method comprises administering to a patient in need thereof an effective amount of a deuterium-enriched compound described herein, such as a compound of Formula I, XII, XIIa, XIIb, XV, or XVIII, to prevent the medical disorder. The medical disorder may be one or more of the medical disorders recited above, such as a neurological disorder (e.g., Alzheimer's disease or Parkinson's disease), cancer (e.g., non-small cell lung cancer or hepatocellular carcinoma), a metabolic disorder, a cardiovascular disorder (e.g. in-stent renarrowing in diabetes patients, reinfarction in diabetes patients, or cardiac allograft vasculopathy after heart transplant), or a respiratory disorder (e.g., chronic obstructive pulmonary disease).

### Veterinary Uses

Also provided are methods of treating veterinary disorders, such as laminitis, using a compound described herein, such as a compound of Formula I, XII, XIIa, XIIb, XV, or XVIII, to treat the veterinary disorder.

### Manufacture of Medicaments

Another aspect of the invention provides for the use of a deuterium-enriched compound described herein in the manufacture of a medicament. The medicament may be for treating one or more of the medical disorders described herein, such as treating a neurological disorder (e.g., Alzheimer's disease or Parkinson's disease), cancer (e.g., non-small cell lung cancer or hepatocellular carcinoma), a metabolic disorder, or a respiratory disorder (e.g., chronic obstructive pulmonary disease).

### Additional Therapeutic Applications

Another aspect of the invention provides an anti-cancer pharmaceutical composition for prophylaxis or treatment of gastric cancer, colon cancer, lung cancer, breast cancer, pancreatic cancer, kidney cancer, prostate cancer, medulloblastoma, rhabdomyosarcoma, Ewing's sarcoma, liposarcoma, multiple myeloma or leukemia, comprising: a pharmaceutically acceptable carrier and a therapeutically effective amount of a deuterium-enriched compound of the invention.

In another aspect, the invention provides an anti-cancer pharmaceutical composition for prophylaxis or treatment of carcinoma, sarcoma or hematopoietic cancer, comprising: a pharmaceutically acceptable carrier, a therapeutically effective amount of a deuterium-enriched compound of the invention, and at least one anti-cancer drug selected from the group consisting of an epidermal growth factor receptor (EGFR) inhibitor (e.g., cetuximab, panitumumab, gefitinib, erlotinib, and lapatinib), a vascular endothelial growth factor receptor (VEGFR) inhibitor (e.g., bevacizumab, sorafenib, SU11248, and vatalanib) and a Raf kinase inhibitor (e.g., sorafenib).

In another aspect, the anti-cancer drug is at least one selected from the group consisting of gefitinib and sorafenib.

In another aspect, the carcinoma is selected from gastric cancer, colon cancer, lung cancer, breast cancer, pancreatic cancer, kidney cancer, and prostate cancer.

In another aspect, the sarcoma is selected from medulloblastoma, rhabdomyosarcoma, Ewing's sarcoma, and liposarcoma.

In another aspect, the hematopoietic cancer is selected from multiple myeloma and leukemia.

In another aspect, the invention provides a method for the prophylaxis or treatment of a person having a carcinoma, sarcoma or hematopoietic cancer, comprising administering to a host in need thereof a therapeutically effective amount of at least one of the deuterium-enriched compounds of the invention or a stereoisomer or pharmaceutically acceptable salt thereof in conjunction with at least one drug selected from an epidermal growth factor receptor (EGFR) inhibitor, a vascular endothelial growth factor receptor (VEGFR) inhibitor, and a Raf kinase inhibitor.

In another aspect, the invention provides a method for prophylaxis or treatment of a cancer selected from: gastric cancer, colon cancer, lung cancer, breast cancer, pancreatic cancer, kidney cancer, prostate cancer, medulloblastoma, rhabdomyosarcoma, Ewing's sarcoma, liposarcoma, multiple myeloma, and leukemia, comprising: administering to a patient in need thereof a therapeutically effective amount of a deuterium-enriched compound of the invention or composition of the invention.

In another aspect, the invention provides a method for prophylaxis or treatment of carcinoma (e.g., gastric cancer, colon cancer, lung cancer, breast cancer, pancreatic cancer, kidney cancer, and prostate cancer), sarcoma (e.g., medulloblastoma, rhabdomyosarcoma, Ewing's sarcoma, and liposarcoma) and hematopoietic cancer (e.g., multiple myeloma and leukemia), comprising: administering to a patient in need thereof a therapeutically effect amount of a deuterium-enriched compound of the invention or composition of the invention.

Epidermal growth factor receptor (EGFR) is a receptor protein that exists on the cell surface corresponding to an epidermal growth factor. The receptor is a membrane-spanning protein, and has a region within the cell where it possesses tyrosine kinase activity. It has become apparent that the receptor is expressed on the surface of many cancer cells, and frequent overexpression is observed especially in lung cancer, breast cancer, colon cancer, pancreatic cancer and the like. With respect to drugs that inhibit the function of epidermal growth factor receptor (EGFR), cetuximab (trade name Erbitux) and panitumumab are examples of antibodies that bind with the extracellular domain. In addition, with respect to inhibitors against tyrosine kinase activity, gefitinib (trade name Iressa), erlotinib (trade name Tarceva) and lapatinib can be mentioned.

Vascular endothelial growth factor receptor (VEGFR) is a receptor protein that exists on the cell surface corresponding to a vascular endothelial growth factor. The receptor is a membrane-spanning protein, and has a region within the cell where it posseses tyrosine kinase activity. It has been known that the receptor is expressed mainly in vascular endothelial cells, and promotes proliferation of vascular endothelial cells by being stimulated with vascular endothelial growth factor secreted from cancer cells. As a result, angiogenesis in the periphery of cancer tissue is enhanced, and proliferation of cancer tissues is promoted. With respect to drugs which inhibit the function of vascular endothelial growth factor receptor (VEGFR), bevacizumab (trade name Avastin), which is a neutralizing antibody against vascular endothelial cell growth factor itself, and sorafenib, SU11248 and vatalanib (PTK787), which are inhibitors against tyrosine kinase activity, are examples.

Raf kinase is one type of serine-threonine kinase, which is deeply involved with cell proliferation signalling, and is known to share a role in a cascade which transduces a proliferation signal from Ras protein, which is a low molecular weight G protein, into a nucleus. With respect to drugs which inhibit kinase activity of Raf, sorafenib is an example.

In another aspect, the invention provides a preventive agent and/or remedy, which comprises a deuterium-enriched compound of the invention or a stereoisomer or pharmaceutically acceptable salt thereof, for diseases alleviated by the above-described actions such as diabetes (e.g., Type I or Type II), hyperlipidemia, obesity, impaired glucose tolerance, hypertension, fatty liver, diabetic complications (e.g. retinopathy, nephropathy, neurosis, cataracts and coronary artery diseases and the like), arteriosclerosis, pregnancy diabetes, polycystic ovary syndrome, cardiovascular diseases (e.g. ischemic heart disease and the like), cell injury (e.g. brain injury induced by strokes and the like) induced by atherosclerosis or ischemic heart disease, gout, inflammatory diseases (e.g. arthrosteitis, pain, pyrexia, rheumatoid arthritis, inflammatory enteritis, acne, sunburn, psoriasis, eczema, allergosis, asthma, GI ulcer, cachexia, autoimmune diseases, pancreatitis and the like), cancer, osteoporosis and cataracts.

In another aspect, the invention provides a pharmaceutical composition comprising a deuterium-enriched compound of the invention or a stereoisomer or a pharmaceutically acceptable salt thereof and at least one compound selected from α-glucosidase inhibitors, aldose reductase inhibitors, biguanide preparations, statin base compounds, squalene synthesis inhibitors, fibrate base compounds, LDL catabolism promoters, and angiotensin-converting enzyme inhibitors.

In another aspect, the invention provides a method of treating a patient, comprising: administering to the patient in need thereof a therapeutically effective amount of a deuterium-enriched compound of the invention or a stereoisomer or pharmaceutically acceptable salt thereof, wherein the treatment is to (i) improve insulin resistance, (ii) reduce inflammation, (iii) achieve immunoregulation, (iv) inhibit aldose reductase, (v) inhibit 5-lipoxygenase, (vi) inhibit lipid peroxide production, (vii) activate peroxisome proliferator activated receptor (PPAR), (viii) provide a leukotriene antagonist, (ix) promote fat-cell-formation, (x) inhibit cancer-cell proliferation, and/or (xi) provide a calcium antagonist.

In another aspect, the invention provides a method of treating a disease, comprising: administering to the patient in need thereof a therapeutically effective amount of a deuterium-enriched compound of the invention or a stereoisomer or pharmaceutically acceptable salt thereof, wherein the disease is selected from: diabetes (e.g., Type I or Type II), hyperlipidemia, impaired glucose tolerance, hypertension, diabetic complications, arteriosclerosis, polycystic ovary syndrome, cardiovascular diseases, atherosclerosis, cell injury induced by ischemic heart diseases, gout, inflammatory diseases, cancer and cataracts.

In another aspect, the invention provides a method of treating a disease, comprising: administering to the patient in need thereof a therapeutically effective amount of a deuterium-enriched compound of the invention or a stereoisomer or pharmaceutically acceptable salt thereof, wherein the treatment is to (i) improve insulin resistance, (ii) reduce inflammation, (iii) achieve immunoregulation, (iv) inhibit aldose reductase, (v) inhibit 5-lipoxygenase, (vi) inhibit lipid peroxide production, (vii) activate peroxisome proliferator activated receptor (PPAR), (viii) provide a leukotriene antagonist, (ix) promote fat-cell-formation, (x) inhibit cancer-cell proliferation, and/or (xi) provide a calcium antagonist, wherein the disease is selected from: diabetes (e.g., Type I or Type II), hyperlipidemia, impaired glucose tolerance, hypertension, diabetic complications, arteriosclerosis, polycystic ovary syndrome, cardiovascular diseases, atherosclerosis, cell injury induced by ischemic heart diseases, gout, inflammatory diseases, cancer and cataracts.

### IV. DOSING CONSIDERATIONS AND COMBINATION THERAPY

Dosages of a compound provided herein, or stereoisomer or pharmaceutically acceptable salt thereof, vary depending on factors such as: specific indication to be treated and/or managed; age and condition of a patient; and amount of second active agent used, if any. Generally, a compound provided herein, or stereoisomer or pharmaceutically acceptable salt thereof, may be used in an amount of from about 0.1 mg to about 500 mg per day, and can be adjusted in a conventional fashion (e.g., the same amount administered each day of the treatment and/or management period), in cycles (e.g., one week on, one week off), or in an amount that increases or decreases over the course of treatment and/or management. In other aspects, the dose can be from about 1 mg to about 300 mg, from about 0.1 mg to about 150 mg, from about 1 mg to about 200 mg, from about 10 mg to about 100 mg, from about 0.1 mg to about 50 mg, from about 1 mg to about 50 mg, from about 10 mg to about 50 mg, from about 20 mg to about 30 mg, or from about 1 mg to about 20 mg.

Unless indicated otherwise, compounds described herein may be administered using any medically accepted route of administration. For example, in certain embodiments, unless indicated otherwise, the compound is administered by oral administration, injection, or transdermal administration. In a preferred embodiment, the compound is administered orally.

### Combination Therapy

A compound provided herein, or a pharmaceutically acceptable salt thereof, can be combined with other pharmacologically active compounds (“second active agents”) in methods and compositions provided herein. Certain combinations may work synergistically in the treatment of particular types of diseases or disorders, and conditions and symptoms associated with such diseases or disorders. A compound provided herein, or a pharmaceutically acceptable salt thereof, can also work to alleviate adverse effects associated with certain second active agents, and vice versa.

One or more second active ingredients or agents can be used in the methods and compositions provided herein. Second active agents can be large molecules (e.g., proteins) or small molecules (e.g., synthetic inorganic, organometallic, or organic molecules).

In certain embodiments, the combination therapy comprises a deuterium-enriched compound described herein and a second therapeutic agent for treating a metabolic disorder, such as metformin, a dipeptidyl peptidase IV inhibitor (e.g., sitagliptin, vildagliptin, or the like), a statin (e.g., a HMG-CoA reductase inhibitor, such as atorvastatin, cerivastatin, fluvastatin, lovastatin, mevastatin, simvastatin, rosuvastatin, pravastatin, or combination thereof), a GLP-1 agonist, a GLP-2 agonist, or an SGLT2 inhibitor. In certain other embodiments, the second therapeutic agent for treating a metabolic disorder is a FBPase inhibitor. As appreciated, the combination therapy may comprising more than two therapeutic agents, such as where a combination of a deuterium-enriched compound described herein and at least two of the aforementioned agents for treating a metabolic disorder are administered to the patient.

In certain other embodiments, the combination therapy comprises a deuterium-enriched compound described herein and a diuretic agent, such as hydrochlorothiazide.

In certain other embodiments, the combination therapy comprises a deuterium-enriched compound described herein and a second therapeutic agent for treating hypertension, diabetes, or an inflammatory disorder. The second therapeutic agent may be one that limits the activity of the renin-angiotensin system, such as an angiotensin converting enzyme inhibitor (e.g., an ACE inhibitor, such as ramipril, captopril, enalapril, or the like), an angiotensin receptor blocker (e.g., candesartan, losartan, olmesartan, or the like), or a renin inhibitor. Alternatively, the second therapeutic agent may limit hypertension by alternate means, such as a beta-adrenergic receptor blocker or calcium channel blocker (e.g., amlodipine).

In certain other embodiments, the combination therapy comprises a deuterium-enriched compound described herein and a glucocorticoid agonist. Such combination therapy may be particularly useful for treating an inflammatory disorder, such as therapy for suppressing an immune response, preventing transplant rejection, and treating autoimmune disease. Exemplary disorders include, for example, rheumatoid arthritis, lupus, myasthenia gravis, muscular dystrophy vasculitis, multiple sclerosis, chronic obstructive pulmonary disease (COPD), inflammatory bowel disease, treatment of acute allergic reactions, and transplant rejection. In certain other embodiments, the combination therapy comprises a deuterium-enriched compound described herein and a second therapeutic agent for treating a kidney disease. Exemplary such second therapeutic agents include those that increase cAMP or comprise a beta-adrenergic agonist. Exemplary beta-adrenergic agonists include, for example, a beta-1-adrenergic agonist, a beta-2-adrenergic agonist, a beta-3-adrenergic agonist, or a combination thereof. In certain embodiments, the second therapeutic agent is noradrenaline, isoprenaline, dobutamine, salbutamol, levosalbutamol, terbutaline, pirbuterol, procaterol, metaproterenol, fenoterol, bitolterol mesylate, salmeterol, formoterol, bambuterol, clenbuterol, indacaterol, L-796568, amibegron, solabegron, isoproterenol, albuterol, metaproterenol, arbutamine, befunolol, bromoacetylalprenololmenthane, broxaterol, cimaterol, cirazoline, denopamine, dopexamine, epinephrine, etilefrine, hexoprenaline, higenamine, isoetharine, isoxsuprine, mabuterol, methoxyphenamine, nylidrin, oxyfedrine, prenalterol, ractopamine, reproterol, rimiterol, ritodrine, tretoquinol, tulobuterol, xamoterol, zilpaterol, zinterol, or a pharmaceutically acceptable salt thereof; or a combination of any of the foregoing.

In certain embodiments, such as when treating an inflammatory disorder, the second therapeutic agent may be, for example, (i) a steroid, such as a corticosteroid, for example beclomethasone (e.g., as the mono or the dipropionate ester), flunisolide, fluticasone (e.g., as the propionate or furoate ester), ciclesonide, mometasone (e.g., as the furoate ester), mometasone, desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocortisone, desoxycorticosterone, etiprednol dicloacetate and the like; (ii) a leukotriene modulator, for example, montelukast or pranlukast; (iii) an anticholinergic agent, for example, a selective muscarinic-3 (M3) receptor antagonist such as ipratropium bromide, tiotropium, or tiotropium bromide; (iv) a phosphodiesterase-IV (PDE-IV) inhibitor, such as roflumilast or cilomilast; (v) an antitussive agent, such as codeine or dextrorphan; (vi) a non-steroidal anti-inflammatory agent (NSAID), such as, ibuprofen or ketoprofen; (vii) a mucolytic, such as, N-acetyl cysteine or fudostein; (viii) a expectorant/mucokinetic modulator, such as, ambroxol, a hypertonic solution (e.g., saline or mannitol) or a surfactant; (ix) a peptide mucolytic, such as, recombinant human deoxyribonuclease I (dornase-alfa and rhDNase) or helicidine; (x) an antibiotic, such as, azithromycin, tobramycin, or aztreonam; or (xi) a p38 MAP kinase inhibitor.

In certain other embodiments, such as when treating an inflammatory disorder, the second therapeutic agent may be, for example, an anti-inflammatory agent in additional to a bronchodilator drug that results in a three-component therapy. Exemplary second and third therapeutic agents include, for example, salmeterol xinafoate/fluticasone propionate, formoterol fumarate/budesonide, formoterol fumarate/mometasone furoate, formoterol fumarate/beclometasone dipropionate, formoterol fumarate/fluticasone propionate, and indacaterol/mometasone furoate. Additional combination therapeutics useful for treating inflammatory disorders include, for example, combinations of a B2 agonist/M3 antagonist, such as salmeterol xinafoate/tiotropium bromide and formoterol fumarate/tiotropium bromide.

In certain other embodiments, the combination therapy comprises a deuterium-enriched compound described herein and a second therapeutic agent for treating cancer. Exemplary second therapeutic agents for treating cancer include, for example, an alkylating agent, an anti-metabolite (i.e., a molecule that impedes DNA and/or RNA synthesis), an anti-microtubule agent, a topoisomerase inhibitor, a cytotoxic antibiotic, a tyrosine kinase inhibitor, an inhibitor of tumor necrosis factor alpha, anti-neoplastic radiation therapy, or a Programmed Death protein-1 (PD-1) modulator (e.g., an inhibitor). In certain embodiments, the second therapeutic agent for treating cancer is azacitidine, azathioprine, bexarotene, bleomycin, carboplatin, capecitabine, carmustine, cetuximab, cisplatin, chlorambucil, cyclophosphamide, cytarabine, dacarbazine, daunorubicin, docetaxel, doxifluridine, doxorubicin, epirubicin, epothilone, erlotinib, etoposide, fluorouracil, fulvestrant, gemcitabine, hydroxyurea, idarubicin, imatinib, lomustine, mechlorethamine, mercaptopurine, methotrexate, mitoxantrone, oxaliplatin, paclitaxel, pemetrexed, procarbazine, raloxifene, teniposide, temozolomide, tamoxifen, toremifene, valrubicin, vinblastine, vincristine, vindesine, vinorelbine, or a pharmaceutically acceptable salt thereof.

In yet other embodiments, the second therapeutic agent for treating cancer is abraxane; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate: bizelesin; bleomycin sulfate; brequinar sodium; bropirimine; busulfan; cactinomycin; calusterone; caracemide; carbetimer; carboplatin; carmustine; carubicin hydrochloride; carzelesin; cedefmgol: celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; portiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; a stem cell treatment; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; or zorubicin hydrochloride.

In certain embodiments, a deuterium-enriched compound described herein is administered in combination with erlotinib to treat cancer, such as non-small cell lung cancer. In certain other embodiments, a deuterium-enriched compound described herein is administered in combination with paclitaxel to treat cancer, such as thyroid cancer (e.g., anaplastic thyroid cancer). In yet other embodiments, a deuterium-enriched compound described herein is administered in combination with bexarotene to treat cancer, such as a solid tumor, a lymphoma, or multiple myeloma.

Administration of a compound provided herein, or a pharmaceutically acceptable salt thereof, and the second active agent(s) to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. One route of administration for compounds provided herein is oral. Routes of administration for the second active agents or ingredients are known to those of ordinary skill in the art. See, e.g., Physicians' Desk Reference (60^{th} Ed., 2006).

In another aspect, the invention provides a method of treating a patient, comprising: administering to the patient in need thereof a therapeutically effective amount of a deuterium-enriched compound of the invention or a stereoisomer or pharmaceutically acceptable salt thereof in combination with at least one compound selected from the group consisting of α-glucosidase inhibitors, aldose reductase inhibitors, biguanide preparations, statin base compounds, squalene synthesis inhibitors, fibrate base compounds, LDL catabolism promoters and angiotensin-converting enzyme inhibitors, wherein the treatment is to (i) improve insulin resistance, (ii) reduce inflammation, (iii) achieve immunoregulation, (iv) inhibit aldose reductase, (v) inhibit 5-lipoxygenase, (vi) inhibit lipid peroxide production, (vii) activate peroxisome proliferator activated receptor (PPAR), (viii) provide a leukotriene antagonist, (ix) promote fat-cell-formation, (x) inhibit cancer-cell proliferation, and/or (xi) provide a calcium antagonist.

An α-glucosidase inhibitor is a medicament having action in inhibiting a digestive enzyme such as amylase, maltase, α-dextrinase or sucrase, thereby retarding the digestion of starch or sucrose. Examples include acarbose, N-(1,3-dihydroxy-2-propyl)variolamine (common name: voglibose) and miglitol.

An aldose reductase inhibitor is a medicament that inhibits a rate-limiting enzyme of the first step of the polyol pathway, thereby inhibiting diabetic complications. Examples include tolrestat, epalrestat, 2,7-difluoro-spiro(9H-fluoren-9,4′-imidazolidine)-2′,5′-dione (common name: imirestat), 3-[(4-bromo-2-fluorophenyl)methyl]-7-chloro-3,4-dihydro-2,4-dioxo-1(2H)-quinozolineacetic acid (common name: zenarestat), 6-fluoro-2,3-dihydro-2,5′-dioxo-spiro[4H-1-benzopyran-4,4′-imidazolidine]-2-carboxamide (SNK-860), zopolrestat, sorbinil, and 1-[(3-bromo-2-benzofuranyl)sulfonyl]-2,4-imidazolidinedione (M-16209).

A biguanide preparation is a medicament having effects in anaerobic glycolysis promotion, insulin action reinforcement at the periphery, intestinal glucose absorption inhibition, hepatic gluconeogenesis inhibition and fatty-acid oxidation inhibition and examples include phenformin, metformin, and buformin.

A statin base compound is a medicament which inhibits hydroxymethylglutaryl CoA (HMG-CoA) reductase, thereby lowering the blood cholesterol level and examples include pravastatin and the sodium salt thereof, simvastatin, lovastatin, atorvastatin, and fluvastatin.

A squalene synthesis inhibitor is a medicament for inhibiting squalene synthesis, thereby lowering the blood cholesterol level and examples include monopotassium (S)-α-[bis(2,2-dimethyl-1-oxopropoxy)methoxy]phosphinyl-3-phenoxybenzenebutanesulfonate (BMS-188494).

A fibrate base compound is a medicament for inhibiting synthesis and secretion of triglycerides in the liver and activating lipoprotein lipase, thereby lowering the triglyceride level in the blood. Examples include bezafibrate, beclobrate, binifibrate, ciprofibrate, clinofibrate, clofibrate, clofibric acid, ethofibrate, fenofibrate, gemfibrozil, nicofibrate, pirifibrate, ronifibrate, simfibrate, and theofibrate.

A LDL catabolism promoter is a medicament for increasing LDL (low-density lipoprotein) receptors, thereby lowering the blood cholesterol level and examples include compounds described in Japanese Patent Application Kokai Hei 7-316144 or salts thereof, more specifically, N-[2-[4-bis(4-fluorophenyl)methyl-1-piperazinyl]ethyl]-7,7-diphenyl-2,4,6-heptatrienoic amide.

The above-described statin base compounds, squalene synthesis inhibitors, fibrate base compounds and LDL catabolism promoters can be replaced with another chemical effective for lowering the blood cholesterol or triglyceride level. Examples of such a medicament include nicotinic acid derivative preparations such as nicomol and niceritrol; antioxidants such as probucol; and ion exchange resin preparations such as cholestyramine.

An angiotensin-converting enzyme inhibitor is a medicament for inhibiting angiotensin-converting enzyme, thereby lowering the blood pressure and at the same time, partially lowering the blood sugar level of a patient suffering from diabetes. Examples include captopril, enalapril, alacepril, delapril, ramipril, lisinopril, imidapril, benazepril, ceronapril, cilazapril, enalaprilat, fosinopril, moveltipril, perindopril, quinapril, spirapril, temocapril, and trandolapril.

In certain other embodiments, such as when treating an inflammatory disorder, a deuterium-enriched compound described herein

### Exemplary Pharmaceutical Compositions

Pharmaceutical compositions can be used in the preparation of individual, single unit dosage forms. Pharmaceutical compositions and dosage forms provided herein comprise a compound provided herein, or a pharmaceutically acceptable salt, solvate, or stereoisomer thereof. Pharmaceutical compositions and dosage forms can further comprise one or more excipients.

Pharmaceutical compositions and dosage forms provided herein can comprise one or more additional active ingredients. Examples of optional second, or additional, active ingredients are described above.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms are used will vary from one another and will be readily apparent to those skilled in the art. See, e.g., Remington's Pharmaceutical Sciences, 18th ed., Mack Publishing, Easton Pa. (1990).

In another aspect of the invention, the pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided are pharmaceutical compositions and dosage forms that contain little, if any, lactose or other mono- or di-saccharides. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In another aspect, lactose-free dosage forms comprise active ingredients, microcrystalline cellulose, pre-gelatinized starch, and magnesium stearate.

Also provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are, in another aspect, packaged using materials known to prevent exposure to water such that they can be included in suitable formulary kits. Examples of suitable packaging include, but are not limited to, hermetically sealed foils, plastics, dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. In another aspect, dosage forms comprise a compound provided herein in an amount of from about 0.10 to about 500 mg. Examples of dosages include, but are not limited to, 0.1, 1, 2, 5, 7.5, 10, 12.5, 15, 17.5, 20, 25, 50, 100, 150, 200, 250, 300, 350, 400, 450, or 500 mg.

In another aspect, dosage forms comprise the second active ingredient in an amount of 1-about 1000 mg, from about 5 to about 500 mg, from about 10 to about 350 mg, or from about 50 to about 200 mg. Of course, the specific amount of the second active agent will depend on the specific agent used, the diseases or disorders being treated or managed, and the amount(s) of a compound provided herein, and any optional additional active agents concurrently administered to the patient.

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In another aspect, the invention provides oral dosage forms that are tablets or capsules, in which case solid excipients are employed. In another aspect, the tablets can be coated by standard aqueous for nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinylpyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in another aspect, present in from about 50 to about 99 weight percent of the pharmaceutical composition or dosage form.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In another aspect, pharmaceutical compositions comprise from about 0.5 to about 15 weight percent of disintegrant, or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a Syloid® silica gel (AEROSIL200, manufactured by W.R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), and mixtures thereof. If used at all, lubricants may be used in an amount of less than about 1 weight percent of the pharmaceutical compositions or dosage forms into which they are incorporated.

In another aspect, the invention provides a solid oral dosage form comprising a compound provided herein, anhydrous lactose, microcrystalline cellulose, polyvinylpyrrolidone, stearic acid, colloidal anhydrous silica, and gelatin.

Active ingredients provided herein can also be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated in its entirety herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents provided herein. In another aspect, the invention procies single unit dosage forms suitable for oral administration such as, but not limited to, tablets, capsules, gelcaps, and caplets that are adapted for controlled-release.

Controlled-release pharmaceutical products improve drug therapy over that achieved by their non-controlled counterparts. In another aspect, the invention provides the use of a controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

In another aspect, the controlled-release formulations are designed to initially release an amount of drug (active ingredient) that promptly produces the desired therapeutic or prophylactic effect, and gradually and continually release of other amounts of drug to maintain this level of therapeutic or prophylactic effect over an extended period of time. In another aspect, in order to maintain a constant level of drug in the body, the drug can be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

Parenteral dosage forms can be administered to patients by various routes including, but not limited to, subcutaneous, intravenous (including bolus injection), intramuscular, and intraarterial. Administration of a parenteral dosage form bypasses patients' natural defenses against contaminants, and thus, in these aspects, parenteral dosage forms are sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and nonaqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms. For example, cyclodextrin and its derivatives can be used to increase the solubility of a compound provided herein. See, e.g., U.S. Pat. No. 5,134,127, which is incorporated in its entirety herein by reference.

Topical and mucosal dosage forms provided herein include, but are limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein are well known to those skilled in the pharmaceutical arts, and depend on the particular tissue to which a given pharmaceutical composition or dosage form will be applied. In another aspect, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are nontoxic and pharmaceutically acceptable. Moisturizers or humectants can also be added to pharmaceutical compositions and dosage forms. Examples of additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Also, the polarity of a solvent carrier, its ionic strength, or tonicity can be adjusted to improve delivery. Compounds such as stearates can also be added to pharmaceutical compositions or dosage forms to alter the hydrophilicity or lipophilicity of one or more active ingredients so as to improve delivery. In other aspects, stearates can serve as a lipid vehicle for the formulation, as an emulsifying agent or surfactant, or as a delivery-enhancing or penetration-enhancing agent. In other aspects, salts, solvates, prodrugs, or stereoisomers of the active ingredients can be used to further adjust the properties of the resulting composition.

In another aspect, the active ingredients provided herein are not administered to a patient at the same time or by the same route of administration. In another aspect, provided are kits which can simplify the administration of appropriate amounts of active ingredients.

In another aspect, the invention provides a kit comprising a dosage form of a compound provided herein. Kits can further comprise additional active ingredients such as oblimersen (Genasense®), melphalan, G-CSF, GM-CSF, EPO, topotecan, dacarbazine, irinotecan, taxotere, IFN, COX-2 inhibitor, pentoxifylline, ciprofloxacin, dexamethasone, IL2, IL8, IL1 8, Ara-C, vinorelbine, isotretinoin, 13-cis-retinoic acid, or a pharmacologically active mutant or derivative thereof, or a combination thereof. Examples of the additional active ingredients include, but are not limited to, those disclosed herein.

In other aspects, the kits can further comprise devices that are used to administer the active ingredients. Examples of such devices include, but are not limited to, syringes, drip bags, patches, and inhalers.

Kits can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The invention may be embodied in other specific forms without departing from the spirit or essential attributes thereof. This invention encompasses all combinations of preferred aspects of the invention noted herein. It is understood that any and all aspects of the invention may be taken in conjunction with any other aspect or aspects to describe additional aspects. It is also to be understood that each individual element of the aspects is intended to be taken individually as its own independent aspect. Furthermore, any element of an aspect is meant to be combined with any and all other elements from any aspect to describe an additional aspect.

### V. DEFINITIONS

The examples provided in the definitions section as well as the remainder of this application are non-inclusive unless otherwise stated. They include but are not limited to the recited examples.

The compounds herein described may have asymmetric centers, geometric centers (e.g., double bond), or both. All chiral, diastereomeric, racemic forms and all geometric isomeric forms of a structure are intended, unless the specific stereochemistry or isomeric form is specifically indicated. Compounds of the invention containing an asymmetrically substituted atom may be isolated in optically active or racemic forms. It is well known in the art how to prepare optically active forms, such as by resolution of racemic forms, by synthesis from optically active starting materials, or through use of chiral auxiliaries. Geometric isomers of olefins, C═N double bonds, or other types of double bonds may be present in the compounds described herein, and all such stable isomers are included in the invention. Specifically, cis and trans geometric isomers of the compounds of the invention may also exist and may be isolated as a mixture of isomers or as separated isomeric forms. All processes used to prepare compounds of the invention and intermediates made therein are considered to be part of the invention. All tautomers of shown or described compounds are also considered to be part of the invention.

“Alkyl” and “alkylene” includes both branched and straight-chain saturated aliphatic hydrocarbon groups having the specified number of carbon atoms. C₁₋₆ alkyl, for example, includes C₁, C₂, C₃, C₄, C₅, and C₆ alkyl groups. Examples of alkyl include methyl, ethyl, n-propyl, i-propyl, n-butyl, s-butyl, t-butyl, n-pentyl, and s-pentyl. Examples of alkylene include methylene, methylmethylene, ethylene, propylene, trimethylene, methylethylene, ethylethylene, tetramethylene, 1-methyltrimethylene, 2-methyltrimethylene, 3-methyltrimethylene, 1,1-dimethylethylene, pentamethylene, 1-methyltetramethylene, 2-methyltetramethylene, 3-methyltetramethylene, 4-methyltetramethylene, propylethylene, 1,1-dimethyltrimethylene, 2,2-dimethyltrimethylene, 3,3-dimethyltrimethylene, hexamethylene, 1-methylpentamethylene, 2-methylpentamethylene, 3-methylpentamethylene, 4-methylpentamethylene, 5-methylpentamethylene, 1,1-dimethyltetramethylene, 2,2-dimethyltetramethylene, 3,3-dimethyltetramethylene, 4,4-dimethyltetramethylene, butylethylene, and isobutylethylene.

“Alkoxy” is an alkyl group as defined above bound to an oxygen (O) atom. Examples include ethoxy and methoxy.

“Alkylthio” is an alkyl group as defined above bound to a sulfur (S) atom. Examples include ethylthio and methylthio.

As noted previously, “alkyl” also includes deuterated alkyl. Each alkyl group contains 2n+1 hydrogen atoms, wherein n=the number of carbon atoms. Deuterated alkyl covers alkyls groups having from 1 to 2n+1 deuteriums. Deuterated C₁₋₆ alkyl, for example, includes C₁ (d₁₋₃), C₂ (d₁₋₅), C₃ (d₁₋₇), C₄ (d₁₋₉), C₅ (d₁₋₁₁), and C₆ (d₁₋₁₃), alkyl groups.

“Haloalkyl” and “haloalkylene” include alkyl and alkylene groups as defined above (including deuteration), wherein one or more hydrogens are replaced by a halogen atom selected from Cl, F, Br, and I. Examples of haloalkyl include trifluoromethyl, 1,1,1-trifluoroethyl, and perfluoroethyl.

“Alkenyl” includes the specified number of hydrocarbon atoms in either straight or branched configuration with one or more unsaturated carbon-carbon bonds that may occur in any stable point along the chain, such as ethenyl and propenyl. C₂₋₆ alkenyl includes C₂, C₃, C₄, C₅, and C₆ alkenyl groups.

As noted previously, “alkenyl” also includes deuterated alkenyl. Each alkenyl group contains 2(n−i)+1 hydrogen atoms, wherein n=the number of carbon atoms and i=number of double bonds. Deuterated alkenyl covers alkenyls groups having from 1 to 2(n−i)+1 deuteriums. Deuterated C₂₋₆ alkenyl, for example, includes C₂ (d₁₋₃), C₃ (d₁₋₅), C₄ (d₁₋₇), C₅ (d₁₋₉), and C₆ (d₁₋₁₁), alkenyl groups.

“Alkynyl” includes the specified number of hydrocarbon atoms in either straight or branched configuration with one or more triple carbon-carbon bonds that may occur in any stable point along the chain, such as ethynyl and propynyl. C₂₋₆ alkynyl includes C₂, C₃, C₄, C₅, and C₆ alkynyl groups.

As noted previously, “alkynyl” also includes deuterated alkynyl. Each alkynyl group contains 2(n−2i)+1 hydrogen atoms, wherein n=the number of carbon atoms and i=number of triple bonds. Deuterated alkenyl covers alkenyls groups having from 1 to 2(n−2i)+1 deuteriums. Deuterated C₂₋₆ alkynyl, for example, includes C₂ (d₁), C₃ (d₁₋₃), C₄ (d₁₋₅), C₅ (d₁₋₇), and C₆ (d₁₋₉), alkynyl groups.

“Cycloalkyl” includes the specified number of hydrocarbon atoms in a saturated ring, such as cyclopropyl, cyclobutyl, cyclopentyl, cyclohexyl, cycloheptyl, and cyclooctyl. C₃₋₈ cycloalkyl includes C₃, C₄, C₅, C₆, C₇, and C₈ cycloalkyl groups.

As noted previously, “cycloalkyl” also includes deuterated cycloalkyl. Each cycloalkyl group contains 2n−1 hydrogen atoms, wherein n=the number of carbon atoms. Deuterated cycloalkyl covers cycloalkyl groups having from 1 to 2n−1 deuteriums. Deuterated C₃₋₈ cycloalkyl, for example, includes C₃ (d₁₋₅), C₄ (d₁₋₇), C₅ (d₁₋₉), C₆ (d₁₋₁₁), C₇ (d₁₋₁₃), and C₈ (d₁₋₁₅), cycloalkyl groups.

“Cycloalkyl carbonyl” refers to a cycloalkyl group as defined above bound to a carbonyl group. For example, C₄₋₁₁ cycloalkylcarbonyl refers to a C₃₋₁₀ cycloalkyl bound to a carbonyl group. Examples include cyclopropanoyl, cyclobutyryl, cyclopentanoyl, cyclohexanoyl, cycloheptylcarbonyl, norbornylcarbonyl and adamantylcarbonyl groups.

“Aryl” refers to any stable 6, 7, 8, 9, 10, 11, 12, or 13 (or the number specified) membered monocyclic, bicyclic, or tricyclic ring, wherein at least one ring, if more than one is present, is aromatic. Examples of aryl include fluorenyl, phenyl, naphthyl, indanyl, and tetrahydronaphthyl.

As noted previously, “aryl” also includes deuterated aryl. For example, phenyl includes d₁₋₅ phenyl.

“Aryloxy” refers to aryl as defined above bound to oxygen (O). Examples include phenoxy, 1-indenyloxy, 2-indenyloxy, 3-indenyloxy, 1-naphthyloxy, and 2-naphthyloxy groups.

“Arylthio” refers to aryl as defined above bound to sulfur (S). For example, C₆₋₁₀ arylthio refers to C₆₋₁₀ aryl defined above group bound to a sulfur atom. Examples include phenylthio, 1-indenylthio, 2-indenylthio, 3-indenylthio, 1-naphthylthio, and 2-naphthylthio groups.

“Aralkyloxy” refers to aryl as defined above bound to alkylene as defined above bound to oxygen (O). Examples of C₇₋₁₆ aralkyloxy include benzyloxy, naphthylmethoxy, indenylmethoxy, diphenylmethoxy, 1-phenethyloxy, 2-phenethyloxy, 1-naphthylethoxy, 2-naphthylethoxy, 1-phenylpropoxy, 2-phenylpropoxy, 3-phenylpropoxy, 1-naphthylpropoxy, 2-naphthylpropoxy, 3-naphthylpropoxy, 1-phenylbutoxy, 2-phenylbutoxy, 3-phenylbutoxy, 4-phenylbutoxy, 1-naphthylbutoxy, 2-naphthylbutoxy, 3-naphthylbutoxy, 4-naphthylbutoxy, 5-phenylpentyloxy, 5-naphthylpentyloxy, 6-phenylhexyloxy, and 6-naphthylhexyloxy groups.

“Aralkyl” refers to aryl as defined above bound to an alkylene as defined above. For example, C₇₋₁₆ aralkyl, refers to C₆₋₁₀ aryl bound to a C₁₋₆ alkylene. Examples include benzyl and 2-phenethyl.

“Aliphatic acyl” refers to a hydrogen, alkyl, alkenyl, or alkynyl (the last three of which are defined above) bound to a carbonyl. For example, C₁₋₇ aliphatic acyl refers to a hydrogen, C₁₋₆ alkyl, C₂₋₆ alkenyl, or C₂₋₆ alkynyl bound to a carbonyl group. Examples include formyl, acetyl, propionyl, butyryl, isobutyryl, valeryl, isovaleryl, pivaloyl, hexanoyl, acryloyl, methacryloyl, and crotonoyl groups.

“Aliphatic acyloxy” refers to a hydrogen, alkyl, alkenyl, or alkynyl (the last three of which are defined above) bound to a carbonyl that is bound to an oxygen (O). Examples include formyloxy, acetoxy, propionyloxy, butyryloxy, isobutyryloxy, valeryloxy, isovaleryloxy, pivaloyloxy, hexanoyloxy, acryloyloxy, methacryloyloxy, and crotonoyloxy groups.

“Aromatic acyl” refers to an aryl as defined above bound to a carbonyl. For example, C₇₋₁₁ aromatic acyl refers to a C₆₋₁₀ aryl bound to a carbonyl group. Examples include benzoyl, 1-indanecarbonyl, 2-indanecarbonyl and 1- or 2-naphthoyl groups.

“Aromatic aliphatic acyl” refers to an aryl as defined above bound to an aliphatic acyl as defined above. For example, C₈₋₁₂ aromatic-aliphatic acyl refers to a phenyl group bound to a C₂₋₆ aliphatic acyl group. Examples include phenylacetyl.

“Heterocycloalkyl” refers to any stable monocyclic, bicyclic, or tricyclic heterocyclic ring that is non-aromatic, and which consists of the specified number of carbon atoms and 1, 2, 3, or 4 heteroatoms independently selected from the group consisting of N, O, and S. If the heterocycloalkyl group is bicyclic or tricyclic, then at least one of the two or three rings must contain a heteroatom, though both or all three may each contain one or more heteroatoms. The N group may be N, NH, or N-substituent, depending on the chosen ring and if substituents are recited. The nitrogen and sulfur heteroatoms may optionally be oxidized (e.g., S, S(O), S(O)₂, and N—O). The heterocycloalkyl ring may be attached to its pendant group at any heteroatom or carbon atom that results in a stable structure. A heterocycloalkyl group can have one or more carbon-carbon double bonds or carbon-heteroatom double bonds in the ring as long as the ring is not rendered aromatic by their presence. The heterocycloalkyl rings described herein may be substituted on carbon or on a nitrogen atom if the resulting compound is stable.

As noted previously, “heterocycloalkyl” also includes deuterated heterocycloalkyl. For example, piperidinyl and piperazino include d₁₋₁₀ piperidinyl or d₁₋₉ piperazino.

Examples of heterocycloalkyl include aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl.

“4-7 Membered saturated nitrogen-containing heterocyclic group” refers to a 4-7 membered saturated heterocyclic group containing at least one nitrogen atom and optionally additional heteroatoms selected from nitrogen, oxygen and sulfur atoms. Examples include azetidinyl, pyrrolidinyl, imidazolidinyl, thiazolidinyl, pyrazolidinyl, piperidinyl, morpholinyl, thiomorpholinyl, piperazinyl, and homopiperazinyl groups.

Examples of 5-6 membered aromatic nitrogen-containing heterocyclic groups include imidazolyl, tetrazolyl, and pyridinyl.

“Heteroaryl” refers to any stable 5, 6, 7, 8, 9, 10, 11, or 12 membered monocyclic, bicyclic, or tricyclic heterocyclic ring that is aromatic, and which consists of carbon atoms and 1, 2, 3, or 4 heteroatoms independently selected from the group consisting of N, O, and S. If the heteroaryl group is bicyclic or tricyclic, then at least one of the two or three rings must contain a heteroatom, though both or all three may each contain one or more heteroatoms. If the heteroaryl group is bicyclic or tricyclic, then only one of the rings must be aromatic. The N group may be N, NH, or N-substituent, depending on the chosen ring and if substituents are recited. The nitrogen and sulfur heteroatoms may optionally be oxidized (e.g., S, S(O), S(O)₂, and N—O). The heteroaryl ring may be attached to its pendant group at any heteroatom or carbon atom that results in a stable structure. The heteroaryl rings described herein may be substituted on carbon or on a nitrogen atom if the resulting compound is stable.

As noted previously, “heteroaryl” also includes deuterated heteroaryl. For example, furanyl or thienyl include d₁₋₃ furanyl or d₁₋₃ thienyl.

Examples of heteroaryl includes acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 2H,6H-1,5,2-dithiazinyl, dihydrofuro[2,3-b]tetrahydrofuran, furanyl, furazanyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, pyranyl, pyrazinyl, pyrazolyl, pyridazinyl, pyridooxazolyl, pyridoimidazolyl, pyridothiazolyl, pyridinyl, pyridyl, pyrimidinyl, pyrrolyl, quinazolinyl, quinolinyl, 4H-quinolizinyl, quinoxalinyl, quinuclidinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“5-6-Membered aromatic nitrogen-containing heterocyclic carbonyl group” refers to a 5-6-membered heteroaryl as defined above, which contains at least one nitrogen atom and optionally additional heteroatoms, bound to a carbonyl group. Examples include pyrrolylcarbonyl, imidazolylcarbonyl, pyrazolylcarbonyl, triazolylcarbonyl, tetrazolylcarbonyl, nicotinoyl, isonicotinoyl, pyrazinylcarbonyl, pyrimidinylcarbonyl, pyridazinylcarbonyl, thiazolylcarbonyl, oxazolylcarbonyl, oxadiazolylcarbonyl and thiadiazolylcarbonyl groups.

Examples of phenyl substituted with R^{a} include 2-, 3- or 4-fluorophenyl, 2-, 3- or 4-chlorophenyl, 2-, 3- or 4-bromophenyl, 2-, 3- or 4-iodophenyl, 2,4-difluorophenyl, 3,5-difluorophenyl, pentafluorophenyl, 3,5-dichlorophenyl, 2-, 3- or 4-hydroxyphenyl, 3,5-dihydroxyphenyl, 2-, 3- or 4-methylphenyl, 2-, 3- or 4-ethylphenyl, 2-, 3- or 4-propylphenyl, 2-, 3- or 4-isopropylphenyl, 2-, 3- or 4-butylphenyl, 2-, 3- or 4-s-butylphenyl, 2-, 3- or 4-t-butylphenyl, 2-, 3- or 4-trifluoromethylphenyl, 2-, 3- or 4-methoxyphenyl, 2-, 3- or 4-ethoxyphenyl, 2-, 3- or 4-propoxyphenyl, 2-, 3- or 4-isopropoxyphenyl, 2-, 3- or 4-butoxyphenyl, 2-, 3- or 4-s-butoxyphenyl, 2-, 3- or 4-t-butoxyphenyl, 2-, 3- or 4-methylthiophenyl, 2-, 3- or 4-ethylthiophenyl, 2-, 3- or 4-isopropylthiophenyl, 2-, 3- or 4-aminophenyl, 3,5-diaminophenyl, 2-, 3- or 4-methylaminophenyl, 2-, 3- or 4-dimethylaminophenyl, 2-, 3- or 4-(N-ethyl-N-methylamino)phenyl, 2-, 3- or 4-diethylaminophenyl, 2-, 3- or 4-(n-pentylamino)phenyl, 2-, 3- or 4-(n-hexylamino)phenyl, 2-, 3- or 4-phenylaminophenyl, 2-, 3- or 4-benzylaminophenyl, 2-, 3- or 4-formylaminophenyl, 2-, 3- or 4-acetylaminophenyl, 2-, 3- or 4-propionylaminophenyl, 2-, 3- or 4-benzoylaminophenyl, 2-, 3- or 4-(2-, 3- or 4-fluorobenzoylamino)phenyl, 2-, 3- or 4-(2-, 3- or 4-chlorobenzoylamino)phenyl, 2-, 3- or 4-(2,4-difluorobenzoylamino)phenyl, 2-, 3- or 4-(4-hydroxy-3,5-dimethylbenzoylamino)phenyl, 2-, 3- or 4-(4-hydroxy-3,5-di-t-butylbenzoylamino)phenyl, 2-, 3- or 4-(1- or 2-naphthoylamino)phenyl, 2-, 3- or 4-phenylacetylaminophenyl, 2-, 3- or 4-(2-, 3- or 4-fluorophenylacetylamino)phenyl, 2-, 3- or 4-(2-, 3- or 4-chlorophenylacetylamino)phenyl, 2-, 3- or 4-(3-phenylpropionylamino)phenyl, 2-, 3- or 4-cyclopentanoylaminophenyl, 2-, 3- or 4-cyclohexanoylaminophenyl, 2-, 3- or 4-nicotinoylaminophenyl, 2-, 3- or 4-isonicotinoylaminophenyl, 2-, 3- or 4-(N-acetyl-N-methylamino)phenyl, 2-, 3- or 4-(N-acetyl-N-pentylamino)phenyl, 2-, 3- or 4-(N-acetyl-N-hexylamino)phenyl, 2-, 3- or 4-(N-benzoyl-N-hexylamino)phenyl, 2-, 3- or 4-(N-3-chlorobenzoyl-N-methylamino)phenyl, 2-, 3- or 4-(N-3-chlorobenzoyl-N-hexylamino)phenyl, 2-, 3- or 4-(N-2,4-difluorobenzoyl-N-hexylamino)phenyl, 2-, 3- or 4-[N-(1- or 2-naphthoyl)-N-hexylamino]phenyl, 2-, 3- or 4-(N-hexyl-N-phenylacetylamino)phenyl, 2-, 3- or 4-(N-isobutyl-N-cycloheptanoyl)amino)phenyl, 2-, 3- or 4-(N-butyl-N-nicotinoylamino)-phenyl, 2-, 3- or 4-cyclopentylphenyl, 2-, 3- or 4-cyclohexylphenyl, 2-, 3- or 4-(1-adamantyl)phenyl, 2-, 3- or 4-biphenylyl, 2-, 3- or 4-(2′-, 3′- or 4′-hydroxy)-biphenylyl, 2-, 3- or 4-(4-hydroxy-3,5-dimethylphenyl)phenyl, 2-, 3- or 4-(4-hydroxy-3,5-diisopropylphenyl)phenyl, 2-, 3- or 4-(3,5-di-t-butyl-4-hydroxyphenyl)phenyl, 2-, 3- or 4-benzylphenyl, 2-, 3- or 4-(4-hydroxybenzyl)phenyl, 2-, 3- or 4-(4-hydroxy-3,5-dimethylbenzyl)phenyl, 2-, 3- or 4-(3,5-di-t-butyl-4-hydroxybenzyl)phenyl, 2-, 3- or 4-phenoxyphenyl, 2-, 3- or 4-(4-hydroxyphenoxy)phenyl, 2-, 3- or 4-(4-hydroxy-3,5-dimethylphenoxy)phenyl, 2-, 3- or 4-(3,5-di-t-butyl-4-hydroxyphenoxy)phenyl, 2-, 3- or 4-benzyloxyphenyl, 2-, 3- or 4-(4-hydroxybenzyloxy)phenyl, 2-, 3- or 4-(4-hydroxy-3,5-dimethylbenzyloxy)phenyl, 2-, 3- or 4-(3,5-di-t-butyl-4-hydroxybenzyloxy)phenyl, 2-, 3- or 4-phenylthiophenyl, 2-, 3- or 4-(4-hydroxyphenylthio)phenyl, 2-, 3- or 4-(3,5-dimethyl-4-hydroxyphenylthio)phenyl, 2-, 3- or 4-(3,5-di-t-butyl-4-hydroxyphenylthio)phenyl, 2-, 3- or 4-formyloxyphenyl, 2-, 3- or 4-acetoxyphenyl, 2-, 3- or 4-propionyloxyphenyl, 2-, 3- or 4-(1-azetidinyl)phenyl 2-, 3- or 4-(1-, 2- or 3-pyrrolidinyl)phenyl, 2-, 3- or 4-(1-, 2-, 3- or 4-piperidinyl)phenyl, 2-, 3- or 4-(2-, 3- or 4-morpholinyl)phenyl, 2-, 3- or 4-(2-, 3- or 4-thiomorpholinyl)phenyl, 2-, 3- or 4-(1- or 2-piperazinyl)phenyl, 2-, 3- or 4-(1-, 2- or 4-imidazolyl)phenyl, 2-, 3- or 4-(teterazol-5-yl)phenyl, 2-, 3- or 4-(2-, 3- or 4-pyridyl)phenyl, 2-, 3- or 4-nitrophenyl, 2-, 3- or 4-cyanophenyl, 2- or 3-chloro-4-hydroxyphenyl, 4-chloro-3,5-dihydroxyphenyl, 3,5-dichloro-4-hydroxyphenyl, 2-fluoro-4-hydroxy-3,5-dimethylphenyl, 3-fluoro-5-hydroxy-2,6-dimethylphenyl, 4-fluoro-3-hydroxy-2,5-dimethylphenyl, 2-chloro-4-hydroxy-3,5-dimethylphenyl, 3-chloro-5-hydroxy-2,6-dimethylphenyl, 4-chloro-3-hydroxy-2,5-dimethylphenyl, 2- or 3-amino-4-chlorophenyl, 2,3-dichloro-4-aminophenyl, 2- or 3-chloro-4-methylaminophenyl, 2-hydroxy-3- or 4-methylphenyl, 2-hydroxy-3,4-dimethylphenyl, 3-hydroxy-4- or 5-methylphenyl, 3-hydroxy-2,4-dimethylphenyl, 4-hydroxy-2- or 3-methylphenyl, 2- or 3-ethyl-4-hydroxyphenyl, 4-hydroxy-2- or 3-propylphenyl, 4-hydroxy-2- or 3-isopropylphenyl, 2- or 3-t-butyl-4-hydroxyphenyl, 4-hydroxy-2,3-dimethylphenyl, 4-hydroxy-2,5-dimethylphenyl, 4-hydroxy-3,5-dimethylphenyl, 3,5-diethyl-4-hydroxyphenyl, 3-t-butyl-4-hydroxy-5-methylphenyl, 4-hydroxy-3,5-dipropylphenyl, 4-hydroxy-3,5-diisopropylphenyl, 2,5-di-t-butyl-4-hydroxyphenyl, 3,5-di-t-butyl-4-hydroxyphenyl, 4-hydroxy-2,3,5-trimethylphenyl, 4-hydroxy-2,3,6-trimethylphenyl, 4-hydroxy-2,3,5,6-tetramethylphenyl, 4-hydroxy-3,5-dimethoxyphenyl, 2- or 3-hydroxy-4-dimethylaminophenyl, 4-benzyl-(2- or 3-hydroxy)phenyl, 3-, 5- or 6-benzyl-2-hydroxyphenyl, 3-hydroxy-4-nitrophenyl, 3-amino-4-methylphenyl, 4-amino-2,3-dimethylphenyl, 4-amino-2,6-dimethylphenyl, 4-amino-3,5-dimethylphenyl, 4-amino-3,5-diethylphenyl, 4-amino-3,5-dipropylphenyl, 4-amino-3,5-diisopropylphenyl, 4-amino-3,5-di-t-butylphenyl, 4-methylamino-3,5-dimethylphenyl, 4-(N-ethyl-N-methylamino)-3,5-dimethylphenyl, 4-acetylamino-3,5-dimethylphenyl, 4-acetylamino-3,5-di-t-butylphenyl, 4-benzoylamino-3,5-dimethylphenyl, 4-acetoxy-3,5-dimethylphenyl, 4-acetoxy-2,3,5-trimethylphenyl, and 3,5-dimethyl-4-nitrophenyl groups.

Examples of pyridyl substituted with R^{a} include 2-, 3- or 4-pyridyl, 3-, 4-, 5- or 6-fluoro-2-pyridyl, 2-, 4-, 5- or 6-fluoro-3-pyridyl, 2- or 3-fluoro-4-pyridyl, 3-, 4-, 5- or 6-chloro-2-pyridyl, 2-, 4-, 5- or 6-chloro-3-pyridyl, 2- or 3-chloro-4-pyridyl, 3-, 4-, 5- or 6-bromo-2-pyridyl, 2-, 4-, 5- or 6-bromo-3-pyridyl, 2- or 3-bromo-4-pyridyl, 3-, 4-, 5- or 6-iodo-2-pyridyl, 2-, 4-, 5- or 6-iodo-3-pyridyl, 2- or 3-iodo-4-pyridyl, 3-, 4-, 5- or 6-hydroxy-2-pyridyl, 2-, 4-, 5- or 6-hydroxy-3-pyridyl, 2- or 3-hydroxy-4-pyridyl, 3-, 4-, 5- or 6-methyl-2-pyridyl, 2-, 4-, 5- or 6-methyl-3-pyridyl, 2- or 3-methyl-4-pyridyl, 3,5-dimethyl-4-pyridyl, 3-, 4-, 5- or 6-ethyl-2-pyridyl, 2-, 4-, 5- or 6-ethyl-3-pyridyl, 2- or 3-ethyl-4-pyridyl, 3,5-diethyl-4-pyridyl, 3-, 4-, 5- or 6-propyl-2-pyridyl, 2-, 4-, 5- or 6-propyl-3-pyridyl, 2- or 3-propyl-4-pyridyl, 3,5-dipropyl-4-pyridyl, 3-, 4-, 5- or 6-isopropyl-2-pyridyl, 2-, 4-, 5- or 6-isopropyl-3-pyridyl, 2- or 3-isopropyl-4-pyridyl, 3,5-diisopropyl-4-pyridyl, 3-, 4-, 5- or 6-t-butyl-2-pyridyl, 2-, 4-, 5- or 6-t-butyl-3-pyridyl, 2- or 3-t-butyl-4-pyridyl, 3,5-di-t-butyl-4-pyridyl, 3-, 4-, 5- or 6-trifluoromethyl-2-pyridyl, 2-, 4-, 5- or 6-trifluoromethyl-3-pyridyl, 2- or 3-trifluoromethyl-4-pyridyl, 3-, 4-, 5- or 6-methoxy-2-pyridyl, 2-, 4-, 5- or 6-methoxy-3-pyridyl, 2- or 3-methoxy-4-pyridyl, 3-, 4-, 5- or 6-ethoxy-2-pyridyl, 2-, 4-, 5- or 6-ethoxy-3-pyridyl, 2- or 3-ethoxy-4-pyridyl, 3-, 4-, 5- or 6-propoxy-2-pyridyl, 2-, 4-, 5- or 6-propoxy-3-pyridyl, 2- or 3-propoxy-4-pyridyl, 3-, 4-, 5- or 6-isopropoxy-2-pyridyl, 2-, 4-, 5- or 6-isopropoxy-3-pyridyl, 2- or 3-isopropoxy-4-pyridyl, 3-, 4-, 5- or 6-t-butoxy-2-pyridyl, 2-, 4-, 5- or 6-t-butoxy-3-pyridyl, 2- or 3-t-butoxy-4-pyridyl, 4-methylthio-2-pyridyl, 6-isopropylthio-3-pyridyl, 6-t-butylthio-2-pyridyl, 3-, 4-, 5- or 6-amino-2-pyridyl, 2-, 4-, 5- or 6-amino-3-pyridyl, 2- or 3-amino-4-pyridyl, 3-, 4-, 5- or 6-methylamino-2-pyridyl, 2-, 4-, 5- or 6-methylamino-3-pyridyl, 2- or 3-methylamino-4-pyridyl, 5-phenylamino-2-pyridyl, 5-benzylamino-2-pyridyl, 5-acetylamino-2-pyridyl, 5-benzoylamino-2-pyridyl, 5-phenylacetylamino-2-pyridyl, 6-phenyl-2-pyridyl, 6-(4-hydroxyphenyl)-2-pyridyl, 6-(4-hydroxy-3,5-dimethylphenyl)-2-pyridyl, 6-(3,5-di-t-butyl-4-hydroxyphenyl)-2-pyridyl, 6-benzyl-2-pyridyl, 6-(4-hydroxybenzyl)-2-pyridyl, 6-(4-hydroxy-3,5-dimethylbenzyl)-2-pyridyl, 6-(3,5-di-t-butyl-4-hydroxybenzyl)-2-pyridyl, 6-phenoxy-2-pyridyl, 6-(4-hydroxyphenoxy)-2-pyridyl, 6-(4-hydroxy-3,5-dimethylphenoxy)-2-pyridyl, 6-(3,5-di-t-butyl-4-hydroxyphenoxy)-2-pyridyl, 6-benzyloxy-2-pyridyl, 6-(4-hydroxybenzyloxy)-2-pyridyl, 6-(4-hydroxy-3,5-dimethylbenzyloxy)-2-pyridyl, 6-(3,5-di-t-butyl-4-hydroxybenzyloxy)-2-pyridyl, 6-phenylthio-2-pyridyl, 6-(4-hydroxyphenylthio)-2-pyridyl, 6-(4-hydroxy-3,5-dimethylphenylthio)-2-pyridyl, 6-(3,5-di-t-butyl-4-hydroxyphenylthio)-2-pyridyl, 3-, 4-, 5- or 6-formyloxy-2-pyridyl, 2-, 4-, 5- or 6-formyloxy-3-pyridyl, 2- or 3-formyloxy-4-pyridyl, 3-, 4-, 5- or 6-acetoxy-2-pyridyl, 2-, 4-, 5- or 6-acetoxy-3-pyridyl, 2- or 3-acetoxy-4-pyridyl, 6-(1-pyrrolidinyl)-2-pyridyl, 6-(1-piperidinyl)-2-pyridyl, 6-(4-morpholinyl)-2-pyridyl, 3-, 4-, 5- or 6-nitro-2-pyridyl, 2-, 4-, 5- or 6-nitro-3-pyridyl, 2- or 3-nitro-4-pyridyl, 5-amino-6-fluoro-2-pyridyl, 5-amino-6-chloro-2-pyridyl, 6-chloro-3-nitro-2-pyridyl, 6-methoxy-5-methyl-3-pyridyl, 6-methyl-2-nitro-3-pyridyl, 6-chloro-3-nitro-2-pyridyl, 6-methoxy-3-nitro-2-pyridyl, 6-isopropoxy-3-nitro-2-pyridyl, 6-t-butoxy-3-nitro-2-pyridyl, and 6-(4-hydroxy-3,5-dimethylphenoxy)-5-nitro-2-pyridyl groups.

Examples of the NH₂ optionally substituted by R^{c} include amino, methylamino, ethylamino, propylamino, isopropylamino, butylamino, s-butylamino, t-butylamino, pentylamino, hexylamino, dimethylamino, diethylamino, N-ethyl-N-methylamino, dipropylamino, dibutylamino, dipentylamino, dihexylamino, phenylamino, 2-, 3- or 4-fluorophenylamino, 2-, 3- or 4-chlorophenylamino, 2-, 3- or 4-bromophenylamino, 2,3-difluorophenylamino, 2,4-difluorophenylamino, 2,4-dichlorophenylamino, 1- or 2-indenylamino, 1- or 2-naphthylamino, diphenylamino, benzylamino, 2-, 3- or 4-fluorobenzylamino, 2-, 3- or 4-chlorobenzylamino, 2-, 3- or 4-bromobenzylamino, 2,3-difluorobenzylamino, 2,4-difluorobenzylamino, 2,4-dichlorobenzylamino, 1- or 2-naphthylmethylamino, 1-indenylmethylamino, 1- or 2-phenethylamino, 1-, 2- or 3-phenylpropylamino, 4-phenylbutylamino, 1-phenylbutylamino, 5-phenylpentylamino, 6-phenylhexylamino, dibenzylamino, formylamino, acetylamino, propionylamino, buturylamino, isobutyrylamino, valerylamino, isovalerylamino, pivaloylamino, hexanoylamino, acryloylamino, methacryloylamino, crotonoylamino, benzoylamino, 1-indanecarbonylamino, 1- or 2-naphthoylamino, 2-, 3- or 4-fluorobenzoylamino, 2-, 3, or 4-chlorobenzoylamino, 2-, 3- or 4-bromobenzoylamino, 2,3-difluorobenzoylamino, 2,4-difluorobenzoylamino, 2,4-dichlorobenzoylamino, 2,6-diisopropylbenzoylamino, 4-trifluoromethylbenzoylamino, 4-hydroxy-3,5-dimethylbenzoylamino, 4-hydroxy-3,5-di-t-butylbenzoylamino, 1-indanecarbonylamino, 1- or 2-naphthoylamino, phenylacetylamino, 3-phenylpropionylamino, 4-phenylbutyrylamino, 5-phenylpentanoylamino, 6-phenylhexanoylamino, 2-, 3- or 4-fluorophenylacetylamino, 2-, 3- or 4-chlorophenylacetylamino, 2-, 3- or 4-bromophenylacetylamino, 2,3-difluorophenylacetylamino, 2,4-difluorophenylacetylamino, 2,4-dichlorophenylacetylamino, cyclopropionylamino, cyclobutyrylamino, cyclopentanoylamino, cyclohexanoylamino, pyrrolylcarbonylamino, imidazolylcarbonylamino, pyrazolylcarbonylamino, triazolylcarbonylamino, tetrazolylcarbonylamino, nicotinoylamino, isonicotinoylamino, pyrazinylcarbonylamino, pyrimidinylcarbonylamino, pyridazinylcarbonylamino, thiazolylcarbonylamino, oxazolylcarbonylamino, oxadiazolylcarbonylamino, thiadiazolylcarbonylamino, N,N-diacetylamino, N-formyl-N-hexylamino, N-acetyl-N-methylamino, N-acetyl-N-ethylamino, N-acetyl-N-propylamino, N-acetyl-N-butylamino, N-acetyl-N-pentylamino, N-acetyl-N-hexylamino, N-benzoyl-N-methylamino, N-benzoyl-N-ethylamino, N-benzoyl-N-propylamino, N-benzoyl-N-butylamino, N-benzoyl-N-pentylamino, N-benzoyl-N-hexylamino, N-benzoyl-N-phenylamino, N-benzyl-N-benzoylamino, N-4-trifluoromethylbenzyl-N-2,4-difluorobenzoylamino, N-2,4-difluorobenzyl-N-nicotinoylamino, N-3-chlorobenzoyl-N-methylamino, N-3-chlorobenzoyl-N-hexylamino, N-3-chlorobenzyl-N-acetylamino, N-2,4-difluorobenzoyl-N-hexylamino, N-2,4-difluorobenzoyl-N-phenylamino, N-2,4-difluorobenzoyl-N-phenylamino, N-4-trifluoromethylbenzoyl-N-butylamino, N-3,5-di-t-butyl-4-hydroxybenzoyl-N-hexylamino, N-hexyl-N-1-naphthoylamino, N-hexyl-N-2-naphthoylamino, N-hexyl-N-phenylacetylamino, N-isobutyl-N-cycloheptanoylamino, N-butyl-N-nicotinoylamino, N-hexyl-N-nicotinoylamino and N-isonicotinoyl-N-hexylamino groups.

“Host” or “patient” typically refers to a human. It also includes other mammals including the equine, porcine, bovine, feline, and canine families.

“Therapeutically effective amount” includes an amount of a compound of the invention that is effective when administered alone or in combination to treat the desired condition or disorder. “Therapeutically effective amount” includes an amount of the combination of compounds claimed that is effective to treat the desired condition or disorder. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antiviral effect, or some other beneficial effect of the combination compared with the individual components.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. These salts can be prepared in situ in the administration vehicle or the dosage form manufacturing process, or by separately reacting a purified compound of the invention in its free base form with a suitable organic or inorganic acid, and isolating the salt thus formed during subsequent purification. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, bisulfonic, carbonic, citric, edetic, ethane disulfonic, ethane-sulfonic, fumaric, glucoheptonic, gluconic, glutamic, glycolic, glycollyarsanilic, hexylresorcinic, hydrobromic, hydrochloric, hydroiodide, hydroxymaleic, hydroxynaphthoic, isethionic, lactic, lactobionic, lauric, lauryl-sulfonic, maleic, malic, mandelic, methanesulfonic, napsylic, naphthylic, nitric, oleic, oxalic, palmitic, pamoic, pantothenic, phenylacetic, phosphoric, polygalacturonic, propionic, salicyclic, stearic, succinic, sulfamic, sulfanilic, sulfuric, tannic, tartaric, toluenesulfonic, and valeric. (See, for example, Berge et al. (1977) “Pharmaceutical Salts”, J. Pharm. Sci. 66:1-19.)

When a compound of the invention is left in air or is recrystallized, it can absorb water or has adsorbed water attached on the surface and sometimes becomes a hydrate. In addition, compounds of the invention can absorb other solvents and form their solvates. Such solvates (including hydrates) are embraced in the invention and are included when reference is made to a compound or a pharmaceutically acceptable salt thereof.

“Treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder.

“Prevent,” “preventing” and “prevention” refer to the prevention of the onset, recurrence or spread of a disease or disorder, or of one or more symptoms thereof.

“Manage,” “managing” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

“Prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Throughout the description, where compositions are described as having, including, or comprising specific components, or where processes and methods are described as having, including, or comprising specific steps, it is contemplated that, additionally, there are compositions of the invention that consist essentially of, or consist of, the recited components, and that there are processes and methods according to the invention that consist essentially of, or consist of, the recited processing steps.

As a general matter, if a variable is not accompanied by a definition, then the previous definition of the variable controls.

### EXAMPLES

The invention now being generally described, will be more readily understood by reference to the following examples, which are included merely for purposes of illustration of certain aspects and embodiments of the invention, and are not intended to limit the invention.

### Example 1

### Preparation of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione

Deuterated inolitazone was prepared by hydrogen/deuterium exchange in perdeuterated methanol (CD₃OD) in the presence of base. The racemic mixture was characterized by NMR and LC/MS-MS to confirm hydrogen/deuterium exchange at the C-5 position of the thiazolidinedione ring and to determine percent deuterium incorporation, respectively.

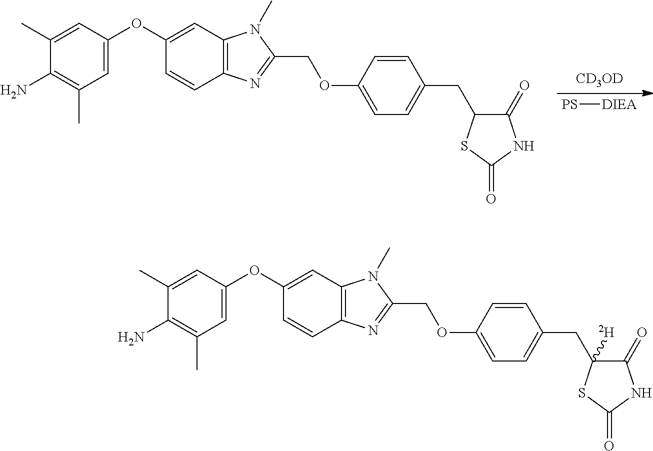

rac-5-[(p-{[6-(4-Amino-3,5-xylyloxy)-1-methyl-1,3-diaza-1H-inden-2-yl]methoxy}phenyl)methyl]-1,3-thiazolidine-2,4-dione (inolitazone, Ontario Chemicals, Inc., Guelph, Ontario, Canada, 125 mg, 0.25 mmol) was dissolved in CD₃OD (2 mL). Diethylamine on polystyrene solid support (PS-DIEA, Biotage, LLC, Charlotte, N.C., 3.68 mmol./g, 0.543 g, 2.0 mmol., 8 eq) was added and the suspension was shaken at room temperature for 24 h. The resin was filtered off, washed with CD₃OD, and the filtrate was evaporated under reduced pressure. The resulting pink solid was dried under high vacuum overnight to give 78.5 mg of title compound (0.16 mmol, 63% yield, 93.7% deuterium content at the C-5 position of the thiazolidinedione ring). ¹H NMR (200 MHz, DMSO-d₆) δ 7.55 (d, J=8 Hz, 1H), 7.14 (d, J=8 Hz, 2H), 7.04 (2 m, 3H), 6.81 (dd, J=8 and 2.5 Hz, 1H), 6.57 (s, 2H), 5.34 (s, 2H), 3.74 (s, 3H), 3.25 (d, J=Hz, 1H), 3.04 (d, J=14 Hz, 1H), 2.06 (s, 6H); MS: [M+1]⁺=504.

The ¹H NMR (200 MHz, DMSO-d₆) of rac-5-[(p-{[6-(4-Amino-3,5-xylyloxy)-1-methyl-1,3-diaza-1H-inden-2-yl]methoxy}phenyl)methyl]-1,3-thiazolidine-2,4 shows a multiplet at 4.90 ppm (dd, J=10, and 4 Hz, 1H) for the hydrogen on C-5 of the thiazolidinedione moiety. This multiplet is absent in the ¹H NMR spectrum of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione. Furthermore, multiplets at 3.30 (dd, 1H)) and 3.10 ppm (dd, 1H) are replaced by two doublets in the ¹H NMR spectrum of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione. Both observations confirm deuterium/hydrogen exchange at the C-5 position of the thiazolidinedione.

### Example 2

### Separation of (+) and (−) enantiomers of rac-5-[(p-{[6-(4-amino-3,5-xylyloxy)-1-methyl-1,3-diaza-1H-inden-2-yl]methoxy}phenyl)methyl]-1,3-thiazolidine-2,4-dione

rac-5-[(p-{[6-(4-Amino-3,5-xylyloxy)-1-methyl-1,3-diaza-1H-inden-2-yl]methoxy}phenyl)methyl]-1,3-thiazolidine-2,4-dione (hrac, 114.7 mg, 0.228 mmol) was dissolved in 70 mL of methanol. The enantiomers were separated by chiral supercritical fluid chromatography (SFC) on a ChiralPak AS-H column (20×250 mm) using a mobile phase of 25% ethanol in carbon dioxide (flow rate: 70 mL/min; 1 mL injected per run). Compounds were detected by UV at 220 nm. Fractions containing the compounds were pooled and evaporated. The purity and enantiomeric excess (% ee=% enantiomer 1−% enantiomer 2) were determined by analytical chiral SFC on a ChiralPak AS-H column (4.6×100 mm) using the same eluent. The enantiomers, peaks 1 and 2, identified as (+)- and (−)-5-[(p-{[6-(4-amino-3,5-xylyloxy)-1-methyl-1,3-diaza-1H-inden-2-yl]methoxy}phenyl)methyl]-1,3-thiazolidine-2,4-dione respectively (optical rotation in methanol at 19.8° C.), were dried under vacuum and stored in the freezer. Yield: 113.4 mg overall (0.226 mmol, 99%) as 55.4 mg peak 1 (0.110 mmol, 96.2% purity, 92.3% ee, and optical rotation=+26.7° (c=0.18, methanol, 19.8° C.)) and 58.0 mg peak 2 (0.116 mmol, 96.0% purity, 92.1% ee, and optical rotation=−60.5° (c=0.21, methanol, 19.8° C.)).

### Example 3

### Separation of (+) and (−) enantiomers of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione

The enantiomers of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione were separated using the same chromatographic method as above. Thus, separation of 68.2 mg (0.136 mmol) of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione gave the two pure enantiomers (combined mass of the two enanteriomes was 56.2 mg (0.112 mmol, 82%) overall yield). The purity and enantiomeric excess were determined by analytical chiral SFC as described above (same retention times for protonated and deuterated enantiomers). Yield: peak 1, (+)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione: 28.6 mg (0.057 mmol, 96.2% purity, 92.4% ee, and optical rotation=+31.7° (c=0.26, methanol, 19.8° C.)); peak 2, (−)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione: 27.6 mg (0.055 mmol, 94.6% purity, 89.2% ee, and optical rotation=−38.5° (c=0.24, methanol, 20.3° C.)).

### Example 4

### Human plasma stability of (+)- and (−)-5-[(p-{[6-(4-amino-3,5-xylyloxy)-1-methyl-1,3-diaza-1H-inden-2-yl]methoxy}phenyl)methyl]-1,3-thiazolidine-2,4-dione and of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione

The (+)-5-[(p-{[6-(4-amino-3,5-xylyloxy)-1-methyl-1,3-diaza-1H-inden-2-yl]methoxy}phenyl)methyl]-1,3-thiazolidine-2,4-dione, (−)-5-[(p-{[6-(4-amino-3,5-xylyloxy)-1-methyl-1,3-diaza-1H-inden-2-yl]methoxy}phenyl)methyl]-1,3-thiazolidine-2,4-dione, and rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione were incubated in human plasma (K₃EDTA as anticoagulant) at 37° C. in duplicates. The aliquot of deuterated enantiomers used for this study was found to contain the deuterated enantiomers in a 0.63:1 (+):(−) ratio. It also contained ˜15% of the protonated enantiomers. Aliquots were removed at time=0, 5, 10, 15, 30 min, and 1, 2, 4, and 8 hours. Plasma proteins were precipitated by addition of acetonitrile containing cisapride as an internal standard (ISTD) and the supernatants were analyzed semi-quantitatively by LC/MS-MS with elution on a chiral column (Daicel ChiralPak IC-3) for the separation of enantiomers (isocratic method of 30:70 v/v 20 mM ammonium bicarbonate in water (pH=9.0) and acetonitrile). Peak areas for the deuterated enantiomers (+)-5-[(p-{[6-(4-amino-3,5-xylyloxy)-1-methyl-1,3-diaza-1H-inden-2-yl]methoxy}phenyl)methyl]-1,3-thiazolidine-2,4-dione and (−)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione were normalized to the ISTD and corrected for the isotopic peak of the corresponding protonated enantiomer, (+)-5-[(p-{[6-(4-amino-3,5-xylyloxy)-1-methyl-1,3-diaza-1H-inden-2-yl]methoxy}phenyl)methyl]-1,3-thiazolidine-2,4-dione and (−)-5-[(p-{[6-(4-amino-3,5-xylyloxy)-1-methyl-1,3-diaza-1H-inden-2-yl]methoxy}phenyl)methyl]-1,3-thiazolidine-2,4-dione, respectively, if present. Corrected data were analyzed and plotted using Microsoft Excel 2013 (Microsoft Corp, Redmond, Wash.) and the Excel Solver.

Scheme 1 illustrates possible reactions in a solution of a mixture of deuterated enantiomers when no degradation or side-reactions occur. The deuterium in both enantiomers, d+ and d−, can be lost by D/H exchange to give both protonated enantiomers, h+ and h− with rate constants k_{D++}, k_{D+-}, k_{D-+}, k_{D--}, where D indicates D/H exchange, the first +/− represents the reagent, d+ or d−, and the second +/− stands for the product, h+ or h−. At the same time, the protonated enantiomers h+ and h− can exchange, with enantiomerization rate constants k₊₋ and k₋₊.

### Scheme 1:

Illustration of possible reactions and corresponding rate constants in a solution of rac-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione where d+, d−, h+, h− stand for H− and (−)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione and (+)- and (−)-5-[(p-{[6-(4-amino-3,5-xylyloxy)-1-methyl-1,3-diaza-1H-inden-2-yl]methoxy}phenyl)methyl]-1,3-thiazolidine-2,4-dione, respectively.

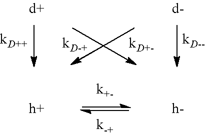

Independent analyses of the data for h+ and h− were performed first and the average rate constants k₊₋ and k₋₊ from these two fits were calculated and used as constants in the fitting of the stability data of the deuterated racemate. Calculated concentrations were obtained through numerical approximation of differential equations (1) and (2) for the stability studies of h+ and h− and equations (3) to (6) for the stability study of the enantiomeric mixture of deuterated d+ and d− by the Euler method (equation (7)). The step between calculated time points was minimized in order to minimize the local error (proportional to the square of the step size) and the global error (proportional to the step size). Data analysis was performed in Microsoft Excel 2013, using the Solver Generalized Reduced Gradient Nonlinear method with central derivatives to minimize the sum of sums of weighted Δ², square of difference between ISTD-normalized experimental data and calculated value, divided by the experimental data.

$$\begin{matrix} \frac{d [ h + ]}{d t} = - {k}_{+ -} [ h + ] + {k}_{- +} [ h - ] \\ \frac{d [ h - ]}{d t} = {k}_{+ -} [ h + ] - {k}_{- +} [ h - ] \\ \frac{d [ h + ]}{d t} = - {k}_{+ -} [ h + ] + {k}_{- +} [ h - ] + {k}_{D ++} [ d + ] + {k}_{D - +} [ d - ] & Equations 1 \text{-} 6 \\ \frac{d [ h - ]}{d t} = {k}_{+ -} [ h + ] - {k}_{- +} [ h - ] + {k}_{D + -} [ d + ] + {k}_{D --} [ d - ] \\ \frac{d [ d + ]}{d t} = - ( {k}_{D ++} + {k}_{D + -} ) [ d + ] \\ \frac{d [ d - ]}{d t} = - ( {k}_{D --} + {d}_{D - +} ) [ d - ] & \end{matrix}$$

where [h+], [h−], [d+], [d−] are the concentrations on both protonated and deuterated enantiomers, k₊₋ and k₋₊ are the rate constants for the enantiomerization reactions [h+] to [h−] and [h−] to [h+] respectively, and k_{D++}, k_{D+-}, k_{D-+}, and k_{D--} are the rate constants for the D/H exchange reactions [d+] or [d−] to [h+] or [h−] respectively.

[X]ₜ₂=[X]ₜ₁+(t₂−t₁)[d[X]]ₜ₁

where [X]ₜᵢ is the concentration of either enantiomer at time ti, t1 is a time at which [X] is known, t2 is a time at which [X] is calculated, and [d[X]]ₜ₁ is the calculated value of the differential equation at time t1.

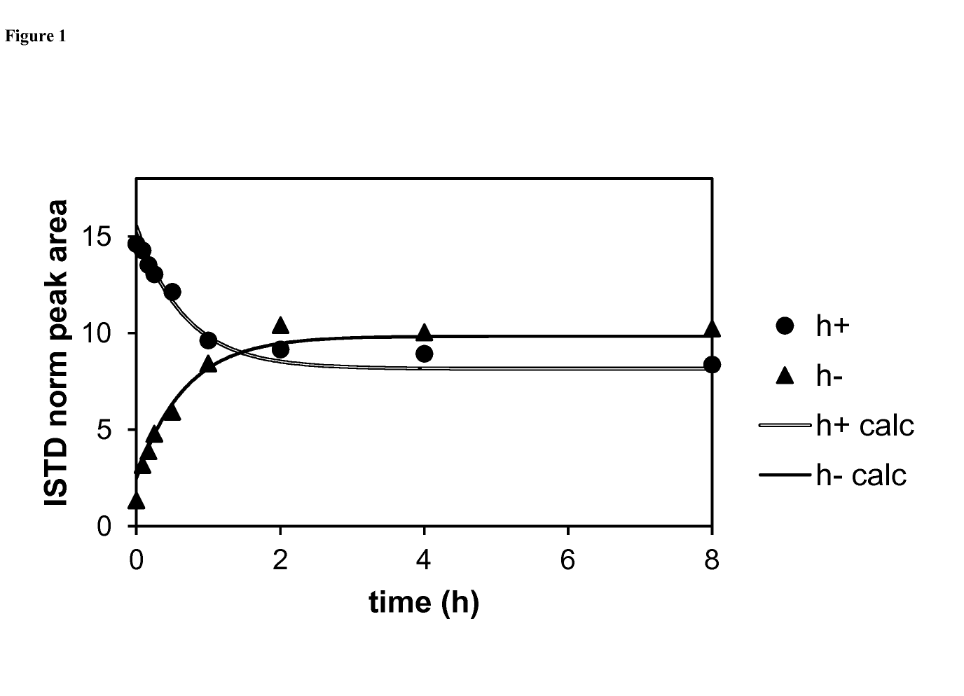

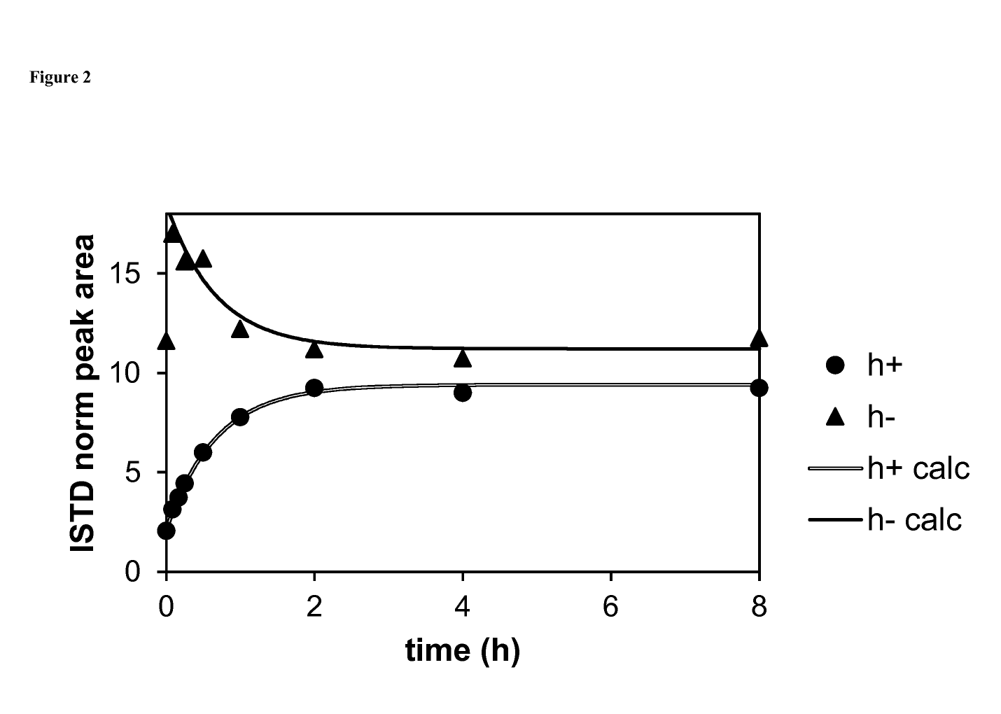

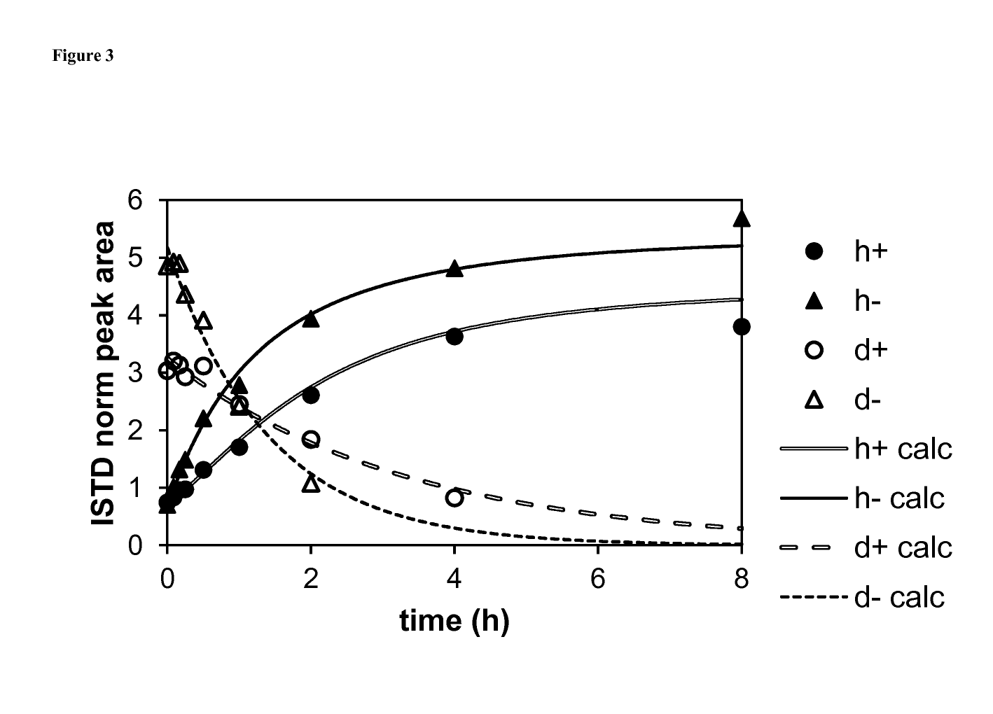

The observed and fitted data are shown in FIGS. 1-3. Fitted parameters are presented in Table 1. The stability of d+ is about 2-3-fold better than that of h+, while the d− enantiomer is not more stable than h−. Dosing d+ or d− would, however, similarly increase exposure to d++h+ and d−+h−, respectively, by a factor 1.5 to 2.0, as predicted by pharmacokinetic simulations using a human elimination half-life of 6 h (Pishvaian M. J. et al, Cancer 118 (2012), 5403-5413).

TABLE 1
Rate constants and calculated half-lives (t_{1/2}) for the in vitro stability of h−, h+,
and a mixture of d− and d+ in human plasma at 37° C. obtained by fitting experimental data
to Equations 1 to 6.

| compound | | d+ → h+ | d+ → h− | d− → h+ | d− → h− | h+ → h−* | h− → h+* |
|---|---|---|---|---|---|---|---|
| d−/d+ | k (h⁻¹) | 0 | 0.300 | 0.211 | 0.499 | 0.802 | 0.668 |
| | t_{1/2} (h) | 2.3 | | 0.98 | | 0.86 | 1.04 |
| h− | k (h⁻¹) | — | — | — | — | 0.800 | 0.671 |
| | t_{1/2} (h) | — | | — | | 0.87 | 1.03 |
| h+ | k (h⁻¹) | — | — | — | — | 0.803 | 0.665 |

| | | | | |
|---|---|---|---|---|
| t_{1/2} (h) | — | — | 0.86 | 1.04 |

*enantiomerization rate constants used in analysis of stability of the mixture of deuterated enantiomers = average of enantiomerization rate constants obtained by fitting data for stability of h− and h+.

### Example 5

### PPARγ agonist activity of (+)- and (−)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione

The agonist activity of (+)- and (−)-5-[(p-{[6-(4-amino-3,5-xylyloxy)-1-methyl-1,3-diaza-1H-inden-2-yl]methoxy}phenyl)methyl]-(5-²H)-1,3-thiazolidine-2,4-dione at the peroxisome proliferator-activated receptor gamma (PPARγ) was evaluated in the thyroid receptor-associated protein complex, 220 kDa component (TRAP220) PPARγ coactivator recruitement assay performed at Cerep (France). Briefly, a mixture of labeled PPARγ and TRAP220 coactivator was pre-incubated with a fluorescence acceptor at room temperature for 30 minutes in the presence of a fluorescence acceptor and the test compound. A fluorescence donor was then added and the mixture was incubated for 120 minutes at room temperature. The fluorescence signal was measured and results expressed as a percent of control (10 μM rosiglitazone). A dose response curve was generated for each enantiomer and the experimental data was analyzed using the log(agonist) vs. response−variable slope (four parameters) nonlinear model in GraphPad Prism 6.0 (GraphPad Software, Inc., La Jolla, Calif.).

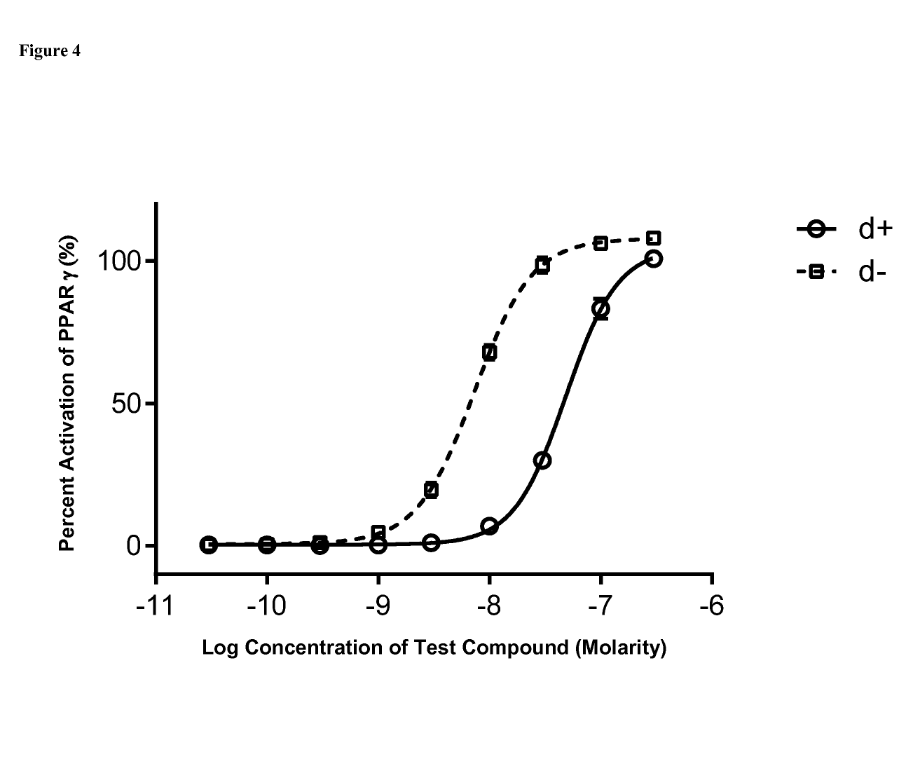

Experimental results are shown in FIG. 4. The results show that deuterated (−) enantiomer, d−, was the most potent (EC₅₀=7.4 nM). Indeed, the deuterated (+) enantiomer, d+, was 7-fold less potent (EC₅₀=48.7 nM) than the (−) enantiomer, d−. Maximum coactivator recruitment was similar for the two enantiomers, and the two deuterated enantiomers were as potent as reference rosiglitazone.

### INCORPORATION BY REFERENCE

All references listed herein are individually incorporated in their entirety by reference.

### EQUIVALENTS

Numerous modifications and variations of the invention are possible in light of the above teachings. It is therefore to be understood that within the scope of the appended claims, the invention may be practiced otherwise that as specifically described herein.

1. A deuterium-enriched compound of formula I: 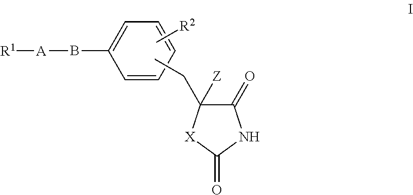
and pharmaceutically acceptable salts and stereoisomers thereof, wherein:
A is C₁₋₆ alkylene;
B is O or S;
X is O or S;
Z is H or D, provided that the abundance of deuterium in Z is at least 30%;
R¹ is selected from: 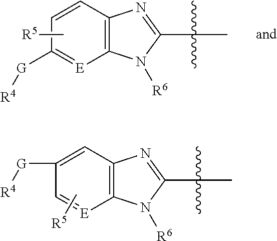
R² is selected from H; D; and R^{a};
E is CH or N;
G is O or S;
R⁴ is phenyl substituted with 1-5 R^{a}, or R⁴ is pyridyl substituted with 1-4 R^{a};
R⁵ is selected from H; D; and R^{a};
R⁶ is selected from H; D; C₁₋₆ alkyl; C₆₋₁₀ aryl group optionally substituted with 1-3 R^{b}; and C₇₋₁₆ aralkyl optionally substituted with 1-3 R^{b};
R^{a} is independently, at each occurrence, selected from:
a. halo;
b. hydroxyl;
c. C₁₋₆ alkyl;
d. halo-C₁₋₆ alkyl;
e. C₁₋₆ alkoxy;
f. C₁₋₆ alkylthio;
g. NH₂ optionally substituted with 1-2 R^{c};
h. C₃₋₁₀ cycloalkyl optionally substituted with 1-3 R^{b};
i. C₆₋₁₀ aryl optionally substituted with 1-3 R^{b};
j. C₇₋₁₆ aralkyl optionally substituted with 1-3 R^{b};
k. C₆₋₁₀ aryloxy optionally substituted with 1-3 R^{b};
l. C₇₋₁₆ aralkyloxy optionally substituted with 1-3 R^{b};
m. C₆₋₁₀ arylthio optionally substituted with 1-3 R^{b};
n. C₁₋₇ aliphatic acyloxy;
o. 4-7 membered saturated nitrogen-containing heterocyclic group;
p. 5-6-membered aromatic nitrogen-containing heterocyclic group;
q. NO₂; and
r. —CN;
R^{b} is independently, at each occurrence, selected from:
a. halo;
b. hydroxyl;
c. C₁₋₆ alkyl;
d. halo-C₁₋₆ alkyl;
e. C₁₋₆ alkoxy;
f. NH₂ optionally substituted with R^{c};
g. C₆₋₁₀ aryl; and
h. NO₂;
R^{c} is independently, at each occurrence, selected from:
a. C₁₋₁₀ alkyl optionally substituted with 1-3 groups R^{d};
b. C₆₋₁₀ aryl optionally substituted with 1-3 groups R^{d};
c. C₇₋₁₆ aralkyl optionally substituted with 1-3 groups R^{d};
d. C₁₋₇ aliphatic acyl optionally substituted with 1-3 groups R^{d};
e. C₇₋₁₁ aromatic acyl optionally substituted with 1-3 groups R^{d};
f. C₈₋₁₂ aromatic aliphatic acyl optionally substituted with 1-3 groups R^{d};
g. C₄₋₁₁ cycloalkylcarbonyl optionally substituted with 1-3 groups R^{d}; and
h. 5-6 membered aromatic nitrogen-containing heterocyclic carbonyl group optionally substituted with 1-3 groups R^{d};
R^{d} is selected from: halogen; hydroxyl; C₁₋₆ alkyl; halo-C₁₋₆ alkyl; C₁₋₆ alkoxy; and C₁₋₆ alkylthio; and
a hydrogen atom present anywhere in the compound of Formula I is optionally replaced by D.

2-10. (canceled)

11. The deuterium-enriched compound of claim 1, wherein the compound is of formula XII or a stereoisomer or pharmaceutically acceptable salt form thereof:

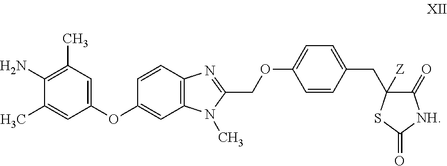

12. The deuterium-enriched compound of claim 11, wherein the abundance of deuterium in Z is at least 90%.

13. The deuterium-enriched compound of claim 11, wherein the abundance of deuterium in Z is at least 95%.

14. The deuterium-enriched compound of claim 13, wherein the compound is a compound of formula XII or stereoisomer thereof.

15. The deuterium-enriched compound of claim 1, wherein the compound is of formula XIIa or pharmaceutically acceptable salt form thereof: 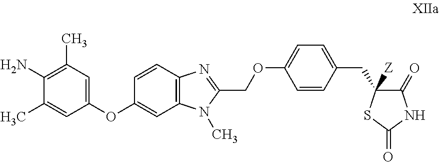
wherein Z is H or D, provided that the abundance of deuterium in Z is at least 50%; and
the compound has an enantiomeric excess, with respect to the C—Z carbon, of at least 80%.

16. The deuterium-enriched compound of claim 15, wherein the abundance of deuterium in Z is at least 80%.

17. The deuterium-enriched compound of claim 15, wherein the abundance of deuterium in Z is at least 95%.

18. The deuterium-enriched compound of claim 16, wherein the compound has an enantiomeric excess, with respect to the C—Z carbon, of at least 90%.

19. The deuterium-enriched compound of claim 17, wherein the compound has an enantiomeric excess, with respect to the C—Z carbon, of at least 95%.

20. The deuterium-enriched compound of claim 19, wherein the compound is a compound of formula XIIa.

21. The deuterium-enriched compound of claim 1, wherein the compound is of formula XIIb or pharmaceutically acceptable salt form thereof: 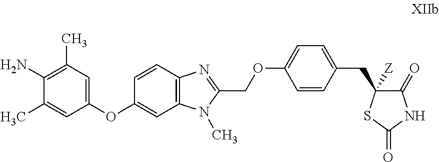
wherein Z is H or D, provided that the abundance of deuterium in Z is at least 50%; and
the compound has an enantiomeric excess, with respect to the C—Z carbon, of at least 80%.

22. The deuterium-enriched compound of claim 21, wherein the abundance of deuterium in Z is at least 80%.

23. The deuterium-enriched compound of claim 21, wherein the abundance of deuterium in Z is at least 95%.

24. The deuterium-enriched compound of claim 22, wherein the compound has an enantiomeric excess, with respect to the C—Z carbon, of at least 90%.

25. The deuterium-enriched compound of claim 23, wherein the compound has an enantiomeric excess, with respect to the C—Z carbon, of at least 95%.

26. The deuterium-enriched compound of claim 25, wherein the compound is a compound of formula XIIb.

27. The deuterium-enriched compound of claim 1, wherein the compound is of formula XII₁ or a stereoisomer or pharmaceutically acceptable salt form thereof:

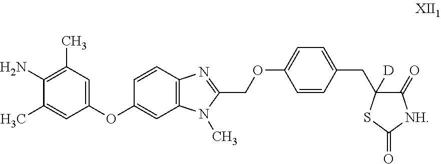 [SMILES: [2H]C1(Cc2ccc(OCc3nc4ccc(Oc5cc(C)c(N)c(C)c5)cc4n3C)cc2)SC(=O)NC1=O]

28. The deuterium-enriched compound of claim 1, wherein the compound is of formula XIIa₁ or pharmaceutically acceptable salt form thereof: 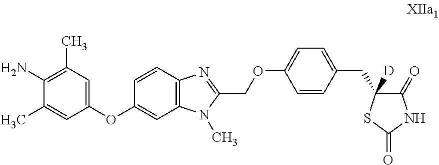 [SMILES: [2H][C@]1(Cc2ccc(OCc3nc4ccc(Oc5cc(C)c(N)c(C)c5)cc4n3C)cc2)SC(=O)NC1=O]
wherein the compound has an enantiomeric excess of at least 80%.

29. The deuterium-enriched compound of claim 28, wherein the compound has an enantiomeric excess of at least 90%.

30. The deuterium-enriched compound of claim 1, wherein the compound is of formula XIIb₁ or pharmaceutically acceptable salt form thereof: 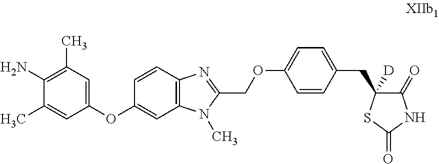 [SMILES: [2H][C@@]1(Cc2ccc(OCc3nc4ccc(Oc5cc(C)c(N)c(C)c5)cc4n3C)cc2)SC(=O)NC1=O]
wherein the compound has an enantiomeric excess of at least 80%.

31. The deuterium-enriched compound of claim 30, wherein the compound has an enantiomeric excess of at least 90%.

32. The deuterium-enriched compound of claim 1, wherein the compound is 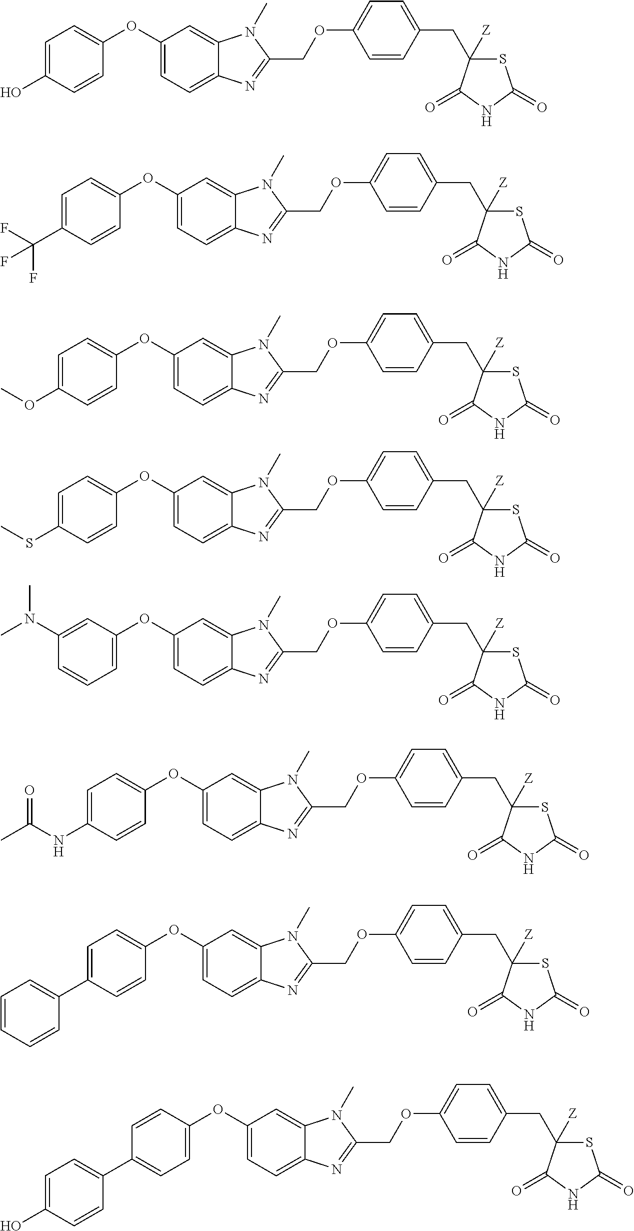 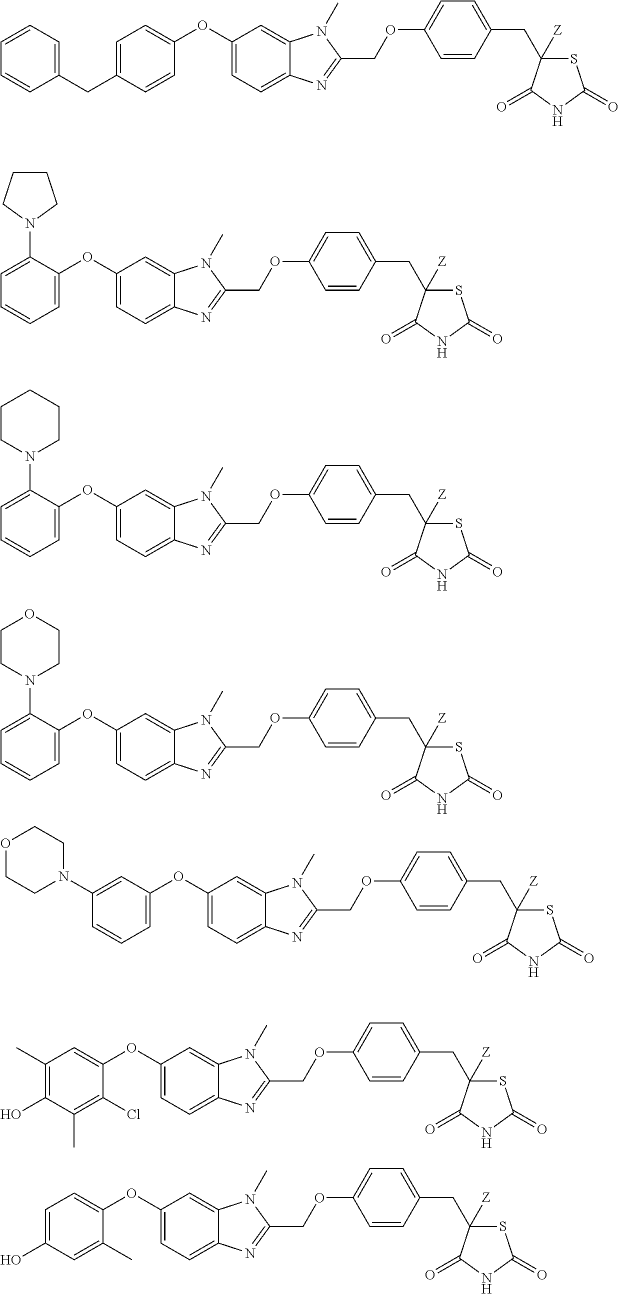 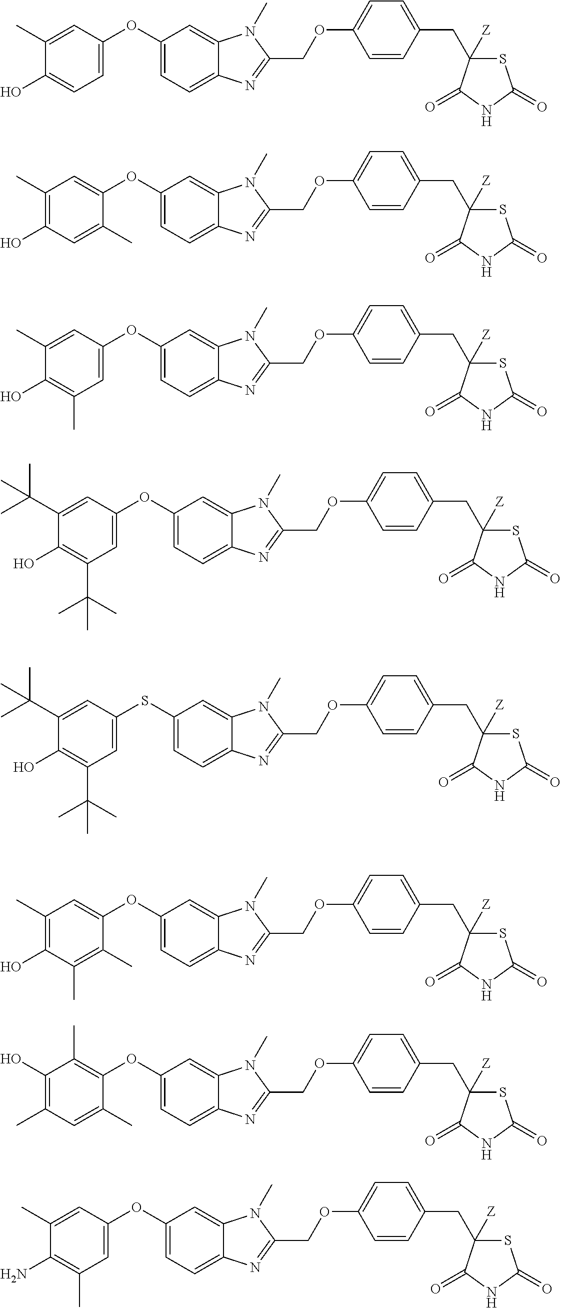 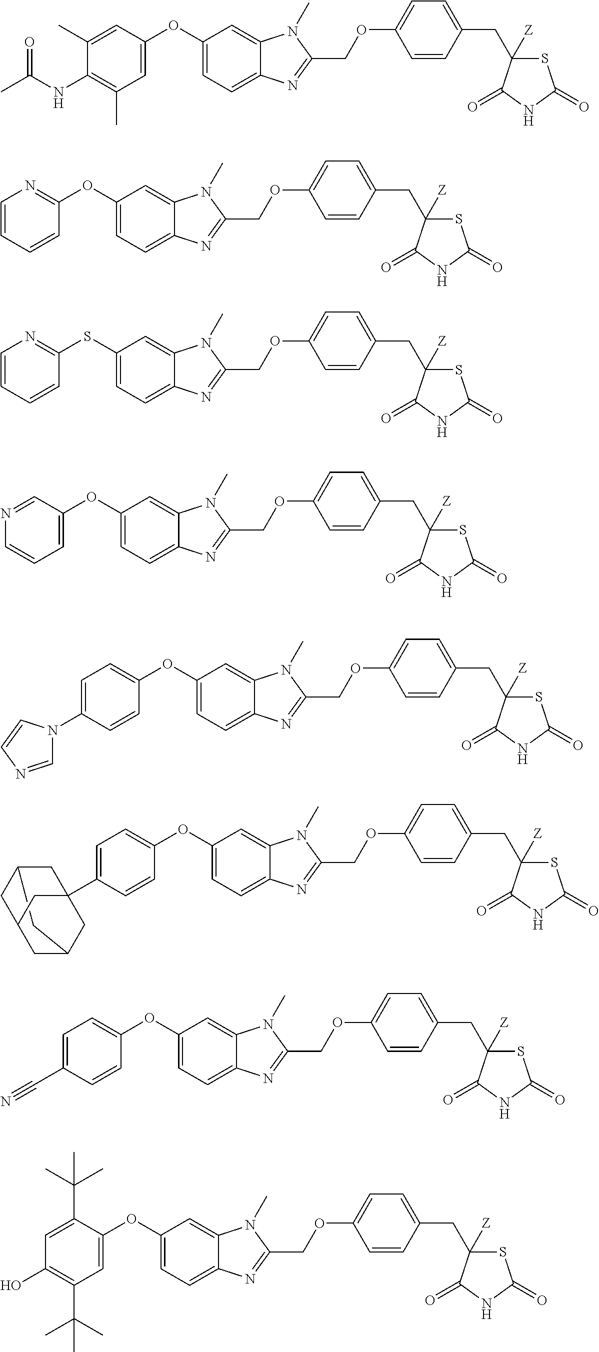 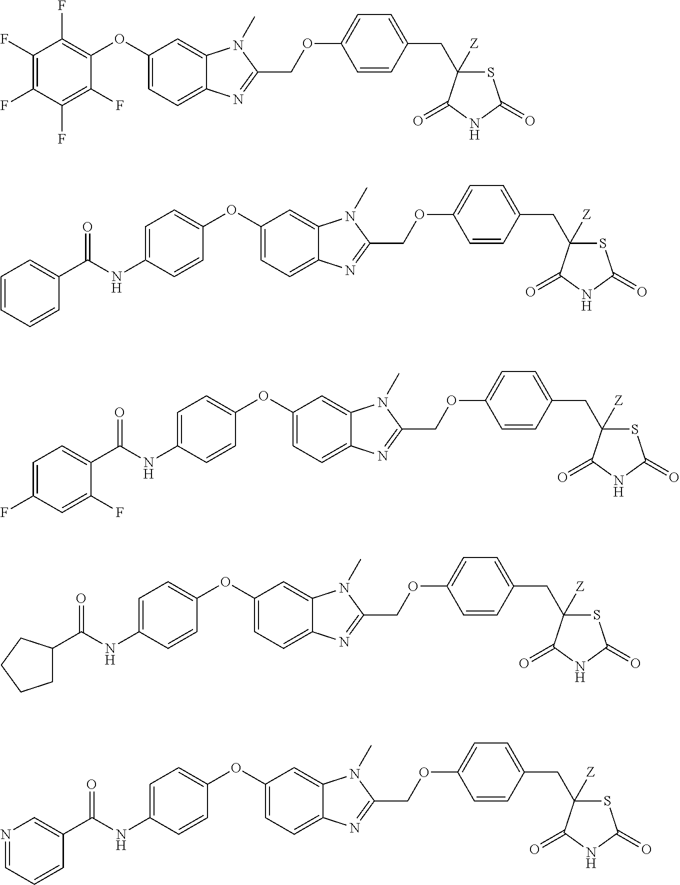
or a stereoisomer or pharmaceutically acceptable salt form thereof.

33-38. (canceled)

39. A pharmaceutical composition comprising a pharmaceutically acceptable carrier and a compound of claim 1.

40. A pharmaceutical composition comprising a pharmaceutically acceptable carrier and a compound of claim 12.

41. A pharmaceutical composition comprising a pharmaceutically acceptable carrier and a compound of claim 18.

42. A pharmaceutical composition comprising a pharmaceutically acceptable carrier and a compound of claim 24.

43. A method for treating a disorder selected from the group consisting of cancer, diabetes, fatty liver disease, and cardiovascular disease in a patient, comprising administering to a patient in need thereof a therapeutically effective amount of a compound of claim 1 to treat the disorder.

44. The method of claim 43, wherein the disorder is cancer.

45. The method of claim 44, wherein the cancer is a carcinoma, sarcoma, or hematopoietic cancer.

46. The method of claim 43, wherein the disorder is diabetes.

47. The method of claim 46, wherein the diabetes is Type II diabetes.
